(19) United States
(12) Patent Application Publication
(10) Pub. No.: US 2024/0261523 A1
(43) Pub. Date: Aug. 8, 2024

(54) INTEGRATION AND MODE SWITCHING FOR RESPIRATORY APPARATUS

(71) Applicant: Fisher & Paykel Healthcare Limited, Auckland (NZ)

(72) Inventors: Aidan Robert BURGESS, Auckland (NZ); Matthew Jon PAYTON, Auckland (NZ); Taylor James EDWARDS, Auckland (NZ); Brittany Diana STEVEN, Auckland (NZ); Craig Karl WHITE, Auckland (NZ); Sam James TULLETT, Auckland (NZ); Mitchell Ryan Cammell OXENBRIDGE, Auckland (NZ); Hamish Adrian OSBORNE, Auckland (NZ); Samantha Dale OLDFIELD, Auckland (NZ); Man Kit Jacky CHEUNG, Auckland (NZ)

(21) Appl. No.: 18/258,897

(22) PCT Filed: Dec. 22, 2021
(86) PCT No.: PCT/IB2021/062153

§ 371 Date: Jun. 22, 2023

Related U.S. Application Data
(60) Provisional application No. 63/129,986, filed on Dec. 23, 2020.
(60) Provisional application No. 63/129,990, filed on Dec. 23, 2020.

(51) Int. Cl. A61M 16/00; A61M 16/01; A61M 16/04; A61M 16/06; A61M 16/08; A61M 16/10; A61M 16/20

(52) U.S. Cl. CPC A61M 16/022; A61M 16/0066; A61M 16/0093; A61M 16/01; A61M 16/04; A61M 16/0666; A61M 16/085; A61M 16/0875; A61M 16/0891; A61M 16/1045; A61M 16/105; A61M 16/202; A61M 16/209; A61M 2016/0033; A61M 2202/0007; A61M 2202/0014; A61M 2202/0208; A61M 2202/0225; A61M 2202/0241; A61M 2202/0275; A61M 2205/3327; A61M 2205/3334; A61M 2205/3379; A61M 2205/36; A61M 2205/502; A61M 2209/08

(57) ABSTRACT

The present disclosure relates to systems and apparatus that provide for integration of different respiratory therapies into a single system, or provide for integrated switching between separate systems or apparatuses delivering different therapies such as anaesthesia and high flow therapy. Aspects of the disclosure relate to various systems, devices, apparatuses, switching mechanisms and multi-lumen lumen assemblies for integrating control of these therapies and providing flexibility in how and where a user controls switching between therapies.

### TECHNICAL FIELD

The present disclosure relates to systems for delivering respiratory support to a patient, and devices and systems that provide for integration of and switching between the function of respiratory apparatuses. It relates particularly but not exclusively to integration and switching between anaesthetic ventilation and high flow modes of respiratory support.

### BACKGROUND OF INVENTION

Patients may lose respiratory function during anaesthesia, or sedation, or more generally during certain medical procedures. Prior to a medical procedure a patient may be pre-oxygenated by a medical professional to provide a reservoir of oxygen saturation, and this pre-oxygenation and CO₂ flushing/washout may be carried out with high flow respiratory support via a nasal cannula or other patient interface.

Once under general anaesthesia, patients must be intubated to ventilate the patient. In some cases, intubation is completed in 30 to 60 seconds, but in other cases, particularly if the patient's airway is difficult to traverse (for example, due to cancer, severe injury, obesity or spasm of the neck muscles), intubation will take significantly longer. While pre-oxygenation provides a buffer against declines in oxygen saturation, for long intubation procedures, it is necessary to interrupt the intubation process and increase the patient's oxygen saturation to adequate levels. The interruption of the intubation process may happen several times for difficult intubation processes, which is time consuming and puts the patient at serious health risk. After approximately three attempts at intubation the medical procedure, such as an intubation method will be abandoned.

High flow systems may be present in the operating theatre for use during anaesthetic or sedation procedures, or other medical procedures. High flow respiratory support has been found effective in meeting or exceeding the patient's normal inspiratory demand, to increase oxygenation of the patient, reduce the work of breathing or perform Transnasal Humidified Rapid-Insufflation Ventilatory Exchange (THRIVE). Pre-oxygenation using high flow systems prior to administration of anaesthesia or sedation provides an oxygen reservoir and extends safe apnoea time. Additionally, high gas flows may generate a flushing effect in the nasopharynx such that the anatomical dead space of the upper airways is flushed by the high incoming gas flows. This creates a reservoir of fresh gas available for each and every breath, while minimising re-breathing of carbon dioxide, nitrogen, etc. THRIVE is the provision of a high flow of respiratory gases to the patient when the patient is apnoeic, which occurs when the anaesthetic agents take effect and before the patient is successfully intubated and mechanically ventilated. High flow respiratory support refers to the delivery of heated and humidified respiratory gases to a patient via a non-sealing patient interface (e.g. nasal cannula) at high flow rates that when the patient is spontaneously breathing, are generally intended to meet or exceed inspiratory demand of a patient.

Once pre-oxygenated, anaesthetic agents are delivered to a patient to sedate the patient prior to intubation. Post intubation, anaesthetic agents are also delivered to maintain the anaesthetized state of the patient during a medical procedure. This delivery of anaesthetic agents can be done via injection or aerosols/vapor-the latter may be achieved by the use of an anaesthesia machine. A system configured for anaesthetic procedure typically includes an anaesthesia machine which includes a rebreathing system in which expired gases from the patient are returned to the machine. The anaesthesia machine provides the anaesthetic agents to sedate the patient and/or keep the patient sedated via a sealing mask placed on the patient. Once sedated, patients are intubated and then mechanically ventilated by the anaesthesia machine (anaesthetic ventilation) that assists or replaces spontaneous breathing.

A reference herein to a patent document or any other matter identified as prior art, is not to be taken as an admission that the document or other matter was known or that the information it contains was part of the common general knowledge as at the priority date of any of the claims.

### SUMMARY OF INVENTION

Currently high flow systems and anaesthetic machines are separate systems and there is no easy, effective and safe way to integrate both systems and/or their functionalities into anaesthetic practise. This creates difficulties in efficiently and safely transitioning from one form of respiratory support to another during medical procedures requiring use of both apparatuses. It would be useful to address or ameliorate one or more of these difficulties.

Viewed from one aspect, the present disclosure provides a system for delivering breathing gases to a patient, the system including:
(a) a first respiratory apparatus configurable to deliver breathing gas including one or more anaesthetic agents to the patient;
(b) a second respiratory apparatus configurable to deliver breathing gas to the patient at a pre-determined flow rate;
(c) a switching means operable to select a mode of operation of the system, said mode of operation being selected from a group including:
   (i) a first mode in which breathing gases are delivered to the patient by the first respiratory apparatus; and
   (ii) a second mode in which breathing gases are delivered to the patient by the second respiratory apparatus.

Typically, in the second mode of operation, the breathing gases delivered to the patient exclude anaesthetic agents.

In some embodiments, when the first mode is selected, the system directs flow of breathing gases in a first inspiratory flow path in which a first patient interface directs breathing gases into an airway of the patient and is a sealing interface. Preferably, the first patient interface directs expired gases from the patient to an expiratory flow path which returns the expired gases to the first inspiratory flow path via the first respiratory apparatus. The first patient interface may be a sealing mask or an endotracheal tube.

In some embodiments, when the second mode is selected, the system isolates flow of breathing gases from the first respiratory apparatus to prevent delivery of anaesthetic agents to the patient. Preferably, when the second mode is selected, the system directs flow of breathing gases in a second inspiratory flow path in which a second patient interface directs the breathing gases into an airway of the patient and is a non-sealing interface such as a nasal cannula.

In some embodiments, the switching means includes a switching mechanism configured to alter flow of breathing gas in the system according to selection of the first mode of operation or the second mode of operation. The switching mechanism may be located between a gases supply and the first and second respiratory apparatuses. In some embodiments, the switching mechanism includes one or more gas flow valves.

In some embodiments, the switching mechanism includes a gas delivery apparatus receiving a supply of gases including NO, O2 and air and having one or more breathing gas outlets. The gas delivery apparatus may include one or more flow meters controlling flow rate of gases comprising one or more of NO, O2 and air through the one or more breathing gas outlets. The one or more flow meters may control flow of breathing gases through the one or more breathing gas outlets responsive to selection of the first mode of operation or the second mode of operation.

In some embodiments, the gas delivery apparatus includes a gas mixing element for combining NO, O2 and air in a proportion required for operation of the system in the first mode.

In some embodiments, the gas delivery apparatus includes a common gas outlet supplying breathing gas from the gas delivery apparatus to the first and second respiratory apparatuses. The gas delivery apparatus may include a first switching element coupled with the switching means and operable to control input to the common gas outlet wherein:
(i) when the first mode is selected, the common gas outlet receives breathing gas from the gas mixing element; and
(ii) when the second mode is selected, the common gas outlet receives breathing gas from the flow meter.

In some embodiments, the first respiratory apparatus and the second respiratory apparatus may be integrated in a unitary machine. The unitary machine may include a humidifier configured to condition the breathing gas to a pre-determined temperature and/or humidity before delivery to the patient in the second mode. The humidifier may be located between the second respiratory apparatuses and the second patient interface.

In some embodiments, the gas delivery apparatus includes a gas mixing element for combining NO, O2 and air in a proportion required for operation of the system in the first mode or the second mode and may further include a flow meter for controlling flow rate of breathing gas from the gas mixing element. The gas delivery apparatus may include:
a first gas outlet supplying breathing gas from the gas delivery apparatus to the first respiratory apparatus;
a second gas outlet supplying breathing gas from the gas delivery apparatus to the second respiratory apparatus; and
a first switching element coupled with the switching means and operable to preclude flow of NO into the gas mixing element when the second mode is selected.

In some embodiments, the system further includes a second switching element coupled with the switching means and operable to control gas flow from the gas delivery apparatus to the first and second breathing apparatuses, wherein (i) when the first mode is selected, breathing gas from the gas delivery apparatus is directed only to the first gas outlet; and (ii) when the second mode is selected, breathing gas from the gas delivery apparatus is directed only to the second gas outlet.

In some embodiments, the gas delivery apparatus includes a first switching element operable to permit flow of breathing gases from the gas delivery apparatus to the first respiratory apparatus when the first mode is selected. The second respiratory apparatus may include a flow meter receiving a supply of gases including O2 for delivery at the predetermined flow rate; and the system may include a second switching element operable to permit flow of breathing gases from the flow meter to the second patient interface when the second mode is selected. The first and second switching elements may be operably coupled such that when the second mode is selected, the first switching element prevents flow of breathing gases from the gas delivery apparatus to the first respiratory apparatus and the second switching element permits flow of breathing gases from the flow meter to the second patient interface.

In some embodiments, the switching mechanism includes a gas diverter receiving a supply of gases including an anaesthetic gas and a breathing gas, the gas diverter having first and second switching elements operable to control flow of gases from the gas diverter to the first and second respiratory apparatuses according to selection of a first mode of operation or a second mode of operation of the system. The first switching element may include a valve controlling flow of anaesthetic gas which is open in the first mode and closed in the second mode. The second switching element may include one or more diverter valves which direct breathing gas to the first respiratory apparatus in the first mode and direct breathing gas to the second respiratory apparatus in the second mode.

In some embodiments, the switching mechanism includes: (a) a first switching element operable to direct flow of O2 to the first respiratory apparatus in the first mode and to the second respiratory apparatus in the second mode; and (b) a second switching element which is responsive to the first switching element, and operable to stop flow of gas from the first apparatus to the patient when the first switching element is operated in the second mode. The first switching element may be e.g. a diverter valve.

In some embodiments, the first respiratory apparatus and the second respiratory apparatus are separate machines. The second respiratory apparatus may include a humidifier configured to condition the breathing gas to a pre-determined temperature and/or humidity before delivery to the patient in the second mode.

In some embodiments, the first switching element and the second switching element are operatively coupled for substantially simultaneous operation with or following operation of the switching means. In other embodiments, the switching means incorporates the first and second switching elements.

In some embodiments, the predetermined flow rate is selectable from an available range of from about 20 L/min to about 90 L/min by the user operating the switching means. In some embodiments, the pre-determined flow rate is selectable by the user from a plurality of pre-determined available flow rates. The pre-determined available flow rates may include at least e.g.: 0 L/min, 40 L/min and 70 L/min.

In some embodiments, the switching means is operable by the user to select delivery of the selected pre-determined flow rate in a continuous flow or oscillating flow. The switching means may include a rate selector operable by the user to control selection and/or delivery of the pre-determined flow rate in the second mode of operation. In some embodiments, operation of the rate selector prevents delivery to the patient of breathing gases from the first respiratory apparatus. In some embodiments, operation of the rate selector to select a rate of 0 L/min permits supply of O2 to the first respiratory apparatus, otherwise operation of the rate selector prevents delivery of anaesthetic agents from the first respiratory apparatus to the patient.

In some embodiments, the switching means includes a pressure-controlled actuator configured to permit flow of breathing gases in the first respiratory apparatus in response to an increase in breathing gas pressure in a flow to the second respiratory apparatus. In some embodiments, the switching means includes a pressure-controlled actuator configured to prevent flow of breathing gases in the first respiratory apparatus in response to a decrease in breathing gas pressure in a flow to the second respiratory apparatus.

In some embodiments, the switching means further provides for selection by the user of a manual first mode of operation or a mechanical first mode of operation of the first respiratory apparatus. For example, the switching means may include a 3-way actuator which provides for selection by the user of manual first mode, mechanical first mode or second mode of operation. Selection of the mechanical first mode may trigger connection by the system of a mechanical ventilator circuit with the first respiratory apparatus. Selection of the manual first mode may trigger connection by the system of a ventilator bag with the first respiratory apparatus.

In some embodiments, selection of the second mode of operation causes the system to substantially simultaneously: (i) prevent flow of breathing gases from the first respiratory apparatus to the patient through a first inspiratory flow path; and (ii) provide a flow of breathing gases from the second respiratory apparatus to the patient through a second inspiratory flow path.

In some embodiments, the system includes a controller receiving inputs from one or more sensors for detecting if a breathing circuit coupled with the patient's airway is associated with a first patient interface for use with the first respiratory apparatus or a second patient interface for use with the second respiratory apparatus, wherein the controller operates the switching means to select the mode of operation according to the detected association of the breathing circuit.

The one or more sensors may include e.g. a pressure sensor arranged to measure back pressure in the first and/or second respiratory apparatuses, wherein the controller determines the breathing circuit to be associated with the first patient interface when the measured back pressure indicates breathing gas is delivered to the patient by a substantially sealed patient interface. Alternatively or additionally, the one or more sensors may include a CO₂ sensor associated with one or each of a first breathing circuit associated with the first patient interface and a second breathing circuit associated with the second patient interface, wherein the controller determines the breathing circuit in which expired gas from the patient contains high concentration of CO₂ to be the breathing circuit through which breathing gas is delivered to the patient. Alternatively or additionally, the sensor may include a proximity sensor.

In some embodiments, the switching means includes one or more actuators operable by a user, the one or more actuators including one or more of: a button, switch, knob, electronic input device, touch screen, voice activated sensor, and foot operated switch. One or more actuators may be located at or near a patient interface through which breathing gas is delivered to the patient by the first respiratory apparatus or the second respiratory apparatus. In some embodiments, the one or more actuators includes an electronic input device wirelessly couplable with a controller of the system and locatable at multiple positions with respect to the patient and/or the first and second breathing apparatuses.

The switching means may include one or more switching mechanisms which may include one or more mechanical, electronic, electromechanical, and pneumatic switching mechanisms. In some embodiments, the one or more switching mechanisms are couplable with the switching means via one or more of wired coupling and wireless coupling. The switching mechanisms may be operable to control one or more characteristics of breathing gases delivered to the subject, said characteristics selected from a group including presence of volatiles, flow rate, gas composition, gas concentration, temperature, and/or humidity.

In some embodiments, the second respiratory apparatus is configured to deliver a flow of breathing gas at the predetermined flow rate being in a range of about 20-90 L/min.

In some embodiments, operation of the system in the second mode precludes delivery of anaesthetic agents in breathing gas delivered to the patient. This may be achieved using any suitable means.

The first respiratory apparatus may include one or more of a CO₂ absorber configured to treat returned expired gas before recirculating to the patient in the first mode, a pressure limiting valve configured to maintain substantially stable pressure in the system in the first mode, a variable volume for displacement of gases in the first mode, a fresh gas flow for replenishing anaesthetic gas delivered to the patient in the first mode and vaporizer for vaporizing volatile anaesthetic agents into breathing gas delivered to the patient in the first mode.

The second respiratory apparatus may include one or more of a flow source configured to generate gas flows through the system and a humidifier configured to condition the breathing gas to a pre-determined temperature and/or humidity before delivery to the patient in the second mode.

Viewed from another aspect, the present disclosure provides a gas delivery device for use with a respiratory system, the gas delivery device including: (a) a first inlet receiving a supply of anaesthetic gas; (b) a second inlet receiving a supply of a breathing gas; (c) a first outlet; (d) a second outlet; and (e) a manifold between the first and second inlets and the first and second outlets, the manifold providing a first flow path to the first outlet and a second flow path to the second outlet; wherein the gas delivery device is operable in: (i) a first configuration the first flow path is open and the second flow path is closed; and (ii) a second configuration in which the second flow path is open and the first flow path is closed.

In some embodiments, in the first configuration, the gas delivery device prevents flow of anaesthetic gas to the outlets. A first valve may control flow of anaesthetic gas in the manifold, wherein the first configuration the first valve is open and directs anaesthetic gas to the first flow path, and in the second configuration the first valve is closed. A second valve may control flow of breathing gas in the manifold, wherein in the first configuration the second valve directs breathing gas to the first flow path, and in the second configuration the second valve directs breathing gas to the second flow path. One or both of the first and second valves may be operable to control rate of flow of gas therethrough.

In some embodiments, the gas delivery device includes a third inlet receiving a supply of a further breathing gas, wherein the breathing gases supplied to the second and third inlets include air and O2. The second valve may be operable to control O₂ concentration in breathing gas delivered to the first and second flow paths.

In some embodiments, the gas delivery device is operable in a third configuration in which both the first flow path and the second flow path are open.

In some embodiments, the gas delivery device includes switching means operable by a user to select a configuration for operation of the gas delivery device. The switching means may be pneumatic, mechanical, electronic or a combination of these.

In some embodiments, the gas delivery device may be configured for connection to a power source. In some embodiments, the gas delivery device may include a battery.

In some embodiments, the first outlet of the gas delivery device may be couplable with a first respiratory apparatus gas inlet. The first respiratory apparatus may be e.g. an anaesthesia machine. In some embodiments, the second outlet of the gas delivery device may be couplable with second respiratory apparatus gas inlet. The second respiratory apparatus may be e.g. a high flow respiratory apparatus.

The gas delivery device may include one or more switching elements for creating the first and second flow paths. A switching element may be located on the first respiratory apparatus. A switching element may be located on the second respiratory apparatus. A switching element may be located on a patient interface through which breathing gas is directed into an airway of the patient. A switching element may be activated responsive to detection of a change in state detected by one or more system sensors. A system sensor may include one of a pressure sensor, CO₂ sensor, O₂ sensor, flow sensor, gas concentration sensor etc. A switching element may be in wired or wireless communication with one or more other switching elements to control operation of the gas delivery device according to the configuration selected by the user.

In some embodiments, the gas delivery device includes an output module providing one or both of a visible and audible indication of the configuration in which the gas delivery device is operating. Operation of the output module may be activated e.g. when the switching means is operated by the user to select the second configuration.

In some embodiments, the gas delivery device includes a gas mixer for combining received gases in a proportion required to deliver a required therapy.

Viewed from another aspect the present disclosure provides a respiratory apparatus operable to deliver breathing gases to a patient in a plurality of modes, the respiratory apparatus providing an inspiratory gas flow path and an expiratory gas flow path, wherein: (a) in a first mode, the respiratory apparatus delivers breathing gases including one or more anaesthetic agents to the inspiratory gas flow path and receives return of expired gases via the expiratory gas flow path; (b) in a second mode, the respiratory apparatus disables flow of one or more anaesthetic agents and delivers breathing gases including O2 to the inspiratory gas flow path at a pre-determined flow rate without return of expired gas; and (c) in a transient mode, the respiratory apparatus disables flow of one or more anaesthetic agents and delivers a high flow of O2 to the inspiratory gas flow path.

The respiratory apparatus may include a switching means operable to select one of the modes of operation from the plurality of modes.

In some embodiments, the transient mode is activated only during operation of an actuator that is normally biased off. The respiratory apparatus may include a button or trigger configured to be activated by a user to operate the respiratory apparatus in the transient mode, wherein release of the button or trigger disables the transient mode.

In some embodiments, in the first mode, the respiratory apparatus is operable to deliver breathing gases including anaesthetic agents to the patient by a first patient interface forming a sealed interface with the patient's airway and returning expired gases to respiratory apparatus by the expiratory gas flow path. The first patient interface may be a mask or endotracheal tube.

In some embodiments, in the second mode, the respiratory apparatus is operable to deliver breathing gases to the patient by a second patient interface forming an unsealed interface with the patient's airway. The second patient interface may be e.g. a nasal canula.

In some embodiments, the respiratory apparatus is operable in a manual first mode of operation or a mechanical first mode of operation. The respiratory apparatus may include a switching means operable by a user for selection of manual first mode, mechanical first mode or second mode of operation. In some embodiments, selection of the manual first mode triggers connection of a manual ventilation bag with the first respiratory apparatus.

In some embodiments, the respiratory apparatus is configurable in gas flow communication with one or more of: a CO2 absorber configured to treat returned expired gas before recirculating to the patient in the first mode; a pressure limiting valve configured to maintain substantially stable pressure in the system in the first mode; a variable volume for displacement of gases in the first mode; a fresh gas flow for replenishing anaesthetic gas delivered to the patient in the first mode; and a vaporizer for vaporizing volatile anaesthetic agents into breathing gas delivered to the patient in the first mode.

In some embodiments, the respiratory apparatus is configurable in gas flow communication with one or more of: a flow source configured to generate gas flows through the system; and a humidifier configured to condition the breathing gas to a pre-determined temperature and/or humidity before delivery to the patient in the second mode.

Viewed from another aspect the present disclosure provides a system for delivering breathing gases to a patient, the system receiving a supply of breathing gases for delivery to the patient by an inspiratory gas flow path and couplable with a returned gas conduit returning expired gases from the patient, the system including: a flow generator configured to generate gas flows through the system; and a switching actuator operable to select a mode of operation of the system; wherein the system is operable in a first mode in which breathing gases are delivered in a closed gas flow circuit in which expired gases are returned to the system for rebreathing by the patient, and in a second mode in which breathing gases are delivered in an open gas flow circuit without rebreathing.

In some embodiments, the flow generator is a blower operable to deliver gas flows at flow rates that are compatible with delivering respiratory therapies including anaesthesia, ventilation and high flow respiratory support. The flow generator may be operable to deliver gas flows in a range of up to about 90 L/min.

In some embodiments, the switching actuator is operatively coupled with the flow generator, and operation of the switching actuator to select the first mode causes operation of the flow generator at a low flow rate such as less than 15 L/min.

In some embodiments, the switching actuator includes or is operatively coupled with a switching mechanism, and in the first mode the switching mechanism permits a first flow of fresh breathing gas to the system and permits return of expired gases to the system, and in the second mode the switching mechanism permits the first flow of fresh breathing gas into the system and prevents return of expired gases to the system. The switching mechanism may include any suitable mechanism such as e.g. a gas flow diverter or a pressure-controlled gas flow diverter.

In some embodiments, the switching mechanism is located upstream of the flow generator.

In some embodiments, the switching mechanism is operatively coupled with the flow generator, and operation of the switching mechanism in the first mode causes operation of the flow generator at a low flow rate.

The second mode may include a ventilator mode and a high flow mode.

In some embodiments, operation of the switching actuator to select high flow mode causes operation of the flow generator at a flow rate sufficient to deliver high flow respiratory support. In some embodiments, operation of the switching actuator to select ventilator mode causes operation of the flow generator at a flow rate consistent with patient ventilation.

In some embodiments, the system is configured to receive a supply of anaesthesia gases for delivery to the patient in the inspiratory gas flow path in the first mode. The system may include a pressure limiting valve to maintain substantially stable gas pressure in the system. The system may be configured to receive a supply of anaesthesia gases downstream of the flow generator or upstream of the flow generator. The returned gas conduit may be couplable with the system downstream of the flow generator.

In some embodiments, the system includes a gas flow reflector which may be configured e.g. to collect anaesthetic gases expired from the patient and return them to the inspiratory gas flow path in a following inhalation phase.

The system may be configured to prevent supply of anaesthesia gases to the system when the second mode is selected.

The system may include a CO₂ absorber configured to treat returned expired gases before recirculating to the inspiratory gas flow path in the first mode. Alternatively or additionally the system may include a humidifier configured to condition the breathing gas to a pre-determined temperature and/or humidity before delivery to the patient in the second mode.

Viewed from another aspect, the present disclosure provides a system for delivering breathing gases to a patient, the system being operable in a first mode and a second mode, wherein the first mode includes a recirculating gases flow between the system and the patient's airway, and the second mode includes a non-recirculating gases flow between the system and the patient's airway, the system including: (a) first module including a first set of respiratory components; and (b) a second module including a second set of respiratory components, the second module configured to cooperate with the first module to switch between the two modes; wherein the system is operable in the first mode when the first module is activated or coupled together with the second module, and the system is operable in the second mode when the first module is inactivated or uncoupled from the second module.

In the first mode, gases delivered to the patient in the recirculating gases flow may include an anaesthetic agent.

The first set of respiratory components may include one or more of a CO₂ absorber configured to treat returned expired gas before recirculating to the patient in the first mode, a pressure limiting valve configured to maintain substantially stable pressure in the system in the first mode, a variable volume for displacement of gases in the first mode, a fresh gas flow for replenishing anaesthetic gas delivered to the patient in the first mode, and a vaporizer for vaporizing volatile anaesthetic agents into breathing gas delivered to the patient in the first mode.

The second set of respiratory components may include one or more of a flow source configured to generate gas flows through the second set of respiratory components, an inspiratory conduit, a patient interface configured to direct gases from the non-recirculating gases flow to the patient's airway, a humidifier configured to condition the breathing gas to a pre-determined temperature and/or humidity before delivery to the patient in the second mode and a filter upstream of the patient interface.

The first module may include a first gases outlet and a first gases inlet, and the second module includes a second gases inlet and a second gases outlet, wherein the first gases outlet is couplable with the second gases inlet and the first gases inlet is couplable with the second gases outlet.

In some embodiments, operation of the system in the first mode permits a first flow of fresh breathing gas to the system and permits return of expired gases to the system, and the second mode permits the first flow of fresh breathing gas into the system and prevents return of expired gases to the system.

In some embodiments, operation of the system in the second mode prevents release of anaesthetic agents from the system. The second mode may include a ventilator mode and a high flow mode.

The second module may be configured to receive a supply of breathing gas independently of the first module.

Viewed from another aspect, the present disclosure provides a system for delivering breathing gases to a patient, the system including: a flow source configured to generate gas flows through the system in a gas delivery circuit; and a switching mechanism forming part of the gas delivery circuit; wherein the gas delivery circuit has an inspiratory gas flow path and an expiratory gas flow path, and the switching mechanism is configured to switch between gas flow paths in the gas delivery circuit according to selection of a first mode of operation in which the inspiratory gas flow path is in fluid communication with a first patient interface, or a second mode of operation in which the inspiratory gas flow path is in fluid communication with a second patient interface.

The first patient interface may form a substantially sealing interface with the patient's airway and receives expiratory gas from the patient. The second patient interface may form a non-sealing interface with the patient's airway.

In some embodiments, the switching mechanism includes one or more of a gas flow diverter, bi-stable switch, pneumatic switch, rotary switch, lever, knob or other actuator which is operable by the user. The switching mechanism may be operable by a user to switch between inspiratory and expiratory gas flows in the gas delivery circuit.

In some embodiments, the system includes one or more sensors configured to monitor one or more characteristics of gas in the gas delivery circuit, and controls operation of the flow generator based on said one or more monitored characteristics. The characteristics may indicate one or more of e.g. flow, pressure and CO2 to name a few. In some embodiments, the system controls operation of the flow source to generate low flows when the one or more sensors indicate breathing gas flow is to the first patient interface. In some embodiments, the system controls operation of the flow generator to generate high flows when the one or more sensors indicate breathing gas flow is to the second patient interface.

In some embodiments, the system is configured to receive a user input to select the first mode of operation in which the flow source generates a low flow rate below a pre-determined flow rate, or the second mode of operation in which the flow source generates a high flow rate at or above the pre-determined flow rate, wherein the switching mechanism operates responsive to flow rate of gas in the inspiratory gas flow path as generated by the flow source. The switching mechanism may direct flow to the first patient interface in response to low flow rate of gas in the inspiratory gas flow path. The switching mechanism may direct flow to the second patient interface in response to high flow rate of gas in the inspiratory gas flow path.

In some embodiments, the switching mechanism is provided by operation of the first and second patient interfaces in which a first mode of delivery is selected when the first and second patient interfaces are applied to the patient simultaneously, and wherein the second mode of delivery is selected when only the second patient interface is applied to the patient. In some embodiments, the first patient interface is a sealing face mask, and the second patient interface is a nasal cannula adapted to operate including while the sealing face mask is applied over the nasal cannula. Thus the first patient interface may be a mask configured to form a sealing interface over the second patient interface which is a nasal cannula. In some embodiments, the sealing face mask is not operable when the sealing face mask is not applied over the nasal cannula and the second mode of delivery is enabled when only the nasal cannula is applied to the patient.

In some embodiments, simultaneous use of the first and second patient interfaces in the first mode, directs inspiratory gas flow to the patient by one or both of the first and second patient interfaces and returns expiratory gas from the first patient interface to the expiratory gas flow path.

In some embodiments, the first patient interface is in fluid communication with an anaesthetic gas reflector.

In some embodiments, the system includes one or more of a CO2 absorber configured to treat returned expired gas before recirculating to the patient in the first mode, a pressure limiting valve configured to maintain substantially stable pressure in the system in the first mode, a variable volume for displacement of gases in the first mode, a fresh gas flow for replenishing anaesthetic gas delivered to the patient in the first mode and a vaporizer for vaporizing volatile anaesthetic agents into breathing gas delivered to the patient in the first mode.

In some embodiments, the system includes one or more of a flow source configured to generate gas flows through the system, and a humidifier configured to condition the breathing gas to a pre-determined temperature and/or humidity before delivery to the patient in the second mode.

Viewed from another aspect, the present disclosure provides a switching mechanism forming part of a gas delivery circuit delivering breathing gases to a patient, the switching mechanism being configured to switch between inspiratory and expiratory gas flow paths in the gas delivery circuit according to selection of a first mode of operation in which the inspiratory gas flow path is in fluid communication with a first patient interface, or a second mode of operation in which the inspiratory gas flow path is in fluid communication with a second patient interface.

In some embodiments, the switching mechanism may be operable by a user, and may include one or more of: a gas flow diverter, pneumatic switch, rotary switch, lever, knob or other actuator which is operable by the user.

In some embodiments, the switching mechanism may operate responsive to a flow rate of gas in the inspiratory flow path, wherein a high flow rate of gas results in selection of the second mode of operation. In some embodiments, the switching mechanism may be configured to operate responsive to a flow rate of gas in the inspiratory flow path, wherein a low flow rate of gas switches the switching mechanism to the first mode of operation and wherein a high flow rate of gas switches the switching mechanism to the second mode of operation.

Viewed from another aspect, the present disclosure provides a multi-lumen assembly for use with a respiratory support system, the multi-lumen assembly having a plurality of conduits including: (a) a first inspiratory conduit having a first conduit inflow end couplable with a first gas outlet of the respiratory support system; (b) a second inspiratory conduit having a second conduit inflow end couplable with a second gas outlet of the respiratory support system; and (c) an expiratory conduit having an expiratory conduit outflow end couplable with an expired gas inlet of the respiratory support system.

The first inspiratory conduit may have a first conduit outflow end couplable with a first patient interface configured to sealingly engage with and direct flow into an airway of the patient. The second inspiratory conduit may have a second conduit outflow end couplable with a second patient interface configured to direct flow into an airway of the patient and is a non-sealing interface.

In some embodiments, at least a length of the plurality of conduits may be arranged co-axially.

A mechanism may be provided retain at least a length of the plurality of conduits in a group. The mechanism may include a webbing arranged between at least pairs of the plurality of conduits at intervals or continuously along at least a length of the plurality of conduits. The webbing may be frangible to facilitate separation of at least a length of one or more of the plurality of conduits from the group. Alternatively or additionally the mechanism may include a sheath applied around the plurality of conduits. Part of the sheath may be removeable. The sheath may provide a smooth outer surface. Alternatively or additionally the mechanism may include one or more retainers configured to retain two or more conduits in the plurality of conduits in a group. The retainers may be slidable along a length of one of more of the conduits in the plurality of conduits.

In some embodiments, the first inspiratory conduit may be configured to deliver breathing gases including an anaesthetic agent to the patient. The expiratory conduit may be configured to return expired gases from the patient to the respiratory support system. The second inspiratory conduit may be configured to deliver breathing gases to the patient at a flow rate between 20 L/min and 90 L/min.

In some embodiments, the multi-lumen assembly may include or cooperate with a flow switching mechanism operable to direct flow of breathing gas into the first inspiratory conduit or into the second inspiratory conduit. The flow switching mechanism may be a flow diverter. The flow switching mechanism may be operable by a user. Alternatively or additionally the flow switching mechanism may be operatively linked with a respiratory support system controller. In some embodiments, the respiratory support system controller controls the respiratory support system to deliver breathing gases to the multi-lumen assembly according to operation of the flow switching mechanism by the user. The respiratory support system controller may control operation of the flow switching mechanism.

In some embodiments, the outflow end of the first inspiratory conduit and an inflow end of the expiratory conduit form a common gas flow pathway defined by a single gas exchange conduit which is couplable with a first patient interface. A taper may be provided to reduce the overall cross section of the multi-lumen assembly in a region of the single gas exchange conduit.

In some embodiments, the multi-lumen assembly includes a patient-end connector for coupling: (a) an outflow end of the first inspiratory conduit with a first patient interface; and (b) an outflow end of the second inspiratory conduit with a second patient interface. The patient end connector may include a switching element operable to switch the connector between a first mode of operation in which the connector directs breathing gas to the first patient interface, and a second mode of operation in which the connector directs breathing gas to the second patient interface. The switching element may be operatively couplable with a respiratory support system controller. Alternatively or additionally, the switching element may be operable by a user, and the controller controls operation of the respiratory support system according to operation of the switching element by the user. In some embodiments, the respiratory support system controller controls operation of the switching element.

In some embodiments, the multi-lumen assembly may include a gas sampling conduit for monitoring one or more characteristics of gas. The characteristics may be used by a respiratory support system controller to determine if the connector is delivering breathing gas to the patient in the first inspiratory conduit or the second inspiratory conduit and the controller may operates the respiratory support system to automatically select a corresponding mode of operation of the respiratory support system.

Viewed from another aspect, the present disclosure provides a breathing gas connector for use with a respiratory support system delivering breathing gases to a patient, the connector having: (a) an inlet port couplable with a gas flow conduit receiving breathing gases from the respiratory support system; (b) a first outlet port couplable with a first gas flow path delivering breathing gas to the patient through a first patient interface; (c) a second outlet port couplable with a second gas flow path delivering breathing gas to the patient through a second patient interface; and (d) a switching mechanism operable to switch the connector between a first mode of operation the in which the connector directs gas from the inlet port to the first outlet port and a second mode of operation in which the connector directs gas from the inlet port to the second outlet port.

In some embodiments, the connector may include an expiratory gases port couplable with an expiratory gas conduit, wherein in the first mode, the switching mechanism directs expired gases in the first gas flow path to the expiratory gases conduit.

The switching mechanism may be operatively couplable with a controller of the respiratory support system, wherein operation of the connector switching mechanism may select: (a) the first mode of operation causes the controller to operate the respiratory support system in a first mode in which breathing gases including an anaesthetic agent are delivered to a gas flow conduit coupled with the connector; (b) the second mode of operation causes the controller to operate the respiratory support system in a second mode in which breathing gases are delivered at a pre-determined flow rate to a gas flow conduit coupled with the connector.

In some embodiments, the connector switching mechanism may be operatively couplable with a controller of the respiratory support system such that operation of the connector switching mechanism to select the second mode of operation causes the controller to prevent flow of anaesthetic agents in the breathing gas.

In some embodiments, the connector may include a sensor detecting one or more characteristics of gas at the first outlet port or the second outlet port, said characteristics being used to determine if the connector is coupled to the patient's airway by a first patient interface or a second patient interface, the sensor providing input to a controller of the respiratory support system which automatically selects a corresponding mode of operation of the respiratory support system. The one or more characteristics include e.g. gas pressure, CO₂ concentration and gas flow rate.

In some embodiments, a sensor wire may be locatable inside a conduit providing a gas flow path between the sensor and the respiratory support system controller.

Viewed from another aspect, the present disclosure provides a controller for use with a respiratory support system delivering breathing gases to a patient via a multi-lumen assembly, the controller including: (a) a control interface operable to receive a selection by a user of a first mode of operation or a second mode of operation of the respiratory support system; (b) a tube assembly input connector for coupling with a patient end of the multi-lumen assembly having a first inspiratory conduit, a second inspiratory conduit and an expiratory conduit; (c) a first flow port couplable with a first flow path delivering breathing gases to the patient in a first mode; and (d) a second flow port couplable with a second flow path delivering breathing gases to the patient in a second mode.

In some embodiments, the controller may include a switching mechanism configured to direct breathing gas from the first inspiratory conduit to the first flow port when the first mode is selected, and to direct breathing gas from the second inspiratory conduit to the second flow port when the second mode is selected.

In some embodiments, the controller may include a third flow port couplable with an expiratory flow path receiving expired gases from the patient in the second mode. The switching mechanism may be configured to direct expired gases to the third flow port when the second mode is selected.

In some embodiments, the controller may include a mounting means configured to releasably attach the controller to a structure located in close proximity to the patient.

In some embodiments, the control interface may be operatively linked with a respiratory support system controller. The respiratory support system controller may control the respiratory support system to deliver breathing gases to a multi-lumen such as described above according to a mode of operation selected using the control interface. In some embodiments, the respiratory support system controller is operable to control operation of the control interface. The controller may be provided for use with the multi-lumen assembly as described above.

Viewed from another aspect, the present disclosure provides a system for delivering breathing gases to a patient, the system being operable to deliver breathing gases in a first mode through a first patient interface, and in a second mode through a second patient interface, the system including: (a) one or more CO₂ sensors configured to detect CO₂ in expired gas from the patient; (b) a switching mechanism for switching between the first and second modes according to detected CO₂; and (c) a system controller receiving an input from the one or more CO₂ sensors; wherein the system controller operates the switching mechanism to select the first mode or the second mode.

In some embodiments, the switching mechanism is operable to switch modes when at least predetermined threshold of CO₂ is detected. The threshold may be e.g. about equivalent to CO₂ concentration of ambient air.

The one or more switching mechanisms includes one or more mechanical, electronic, electromechanical, and pneumatic switching mechanisms. The one or more switching mechanisms may couplable with the system controller via a wired coupling and/or a wireless coupling.

In some embodiments, in the first mode breathing gases are delivered to the patient's airway through the first patient interface which may be a sealing interface such as e.g. a sealing mask or endotracheal tube, and expired gases are returned to the system by an expiratory gas flow path. The first mode may include a rebreathing mode in which expired gases returned to the system are recirculated for delivery to the patient via the first patient interface. The breathing gases may include one or more anaesthetic agents. In some embodiments, the CO₂ sensor detects CO₂ in expired gases in the expiratory gas flow path.

In some embodiments, the second mode is a high flow mode wherein breathing gases are delivered to the patient at a predetermined flow rate through the second patient interface, which is a non-sealing interface such as e.g. a nasal cannula. The CO₂ sensor may detect CO₂ in expired gases at the second patient interface as they exit the patient's airway. In some embodiments, the CO₂ sensor may be located on the nasal cannula to detect CO₂ in expired gas exiting one or both of the patient's nares.

In some embodiments, the system includes a first respiratory apparatus delivering breathing gases in the first mode, and a second respiratory apparatus delivering breathing gases in the second mode. When the second mode is selected, the system may isolate flow of breathing gases from the first respiratory apparatus to the patient. In some embodiments the first respiratory apparatus and the second respiratory apparatus may be integrated in a unitary machine although that need not be the case. In some embodiments, the system may include a humidifier configured to condition the breathing gas to a pre-determined temperature and/or humidity before delivery to the patient in the second mode.

In some embodiments, the second respiratory apparatus delivers breathing gases at a predetermined flow rate and optionally, the predetermined flow rate may be selectable from an available range of from about 20 L/min to about 90 L/min by operating the switching means.

In some embodiments, the system includes a CO₂ sensor associated with one or each of a first breathing circuit for delivery of breathing gases in the first mode and a second breathing circuit for delivery of breathing gases in the second mode. The controller may determine the breathing circuit containing highest concentration of CO₂ to be the breathing circuit through which breathing gas is to be delivered to the patient and controls delivery of gases to the determined breathing circuit according to the relevant mode of operation.

In some embodiments, the system includes a display device in operative communication with the system controller and configurable to display one or more C CO₂ O2 traces based on inputs from the one or more CO₂ sensors received by the system controller. The system controller may be configured to automatically cause display of a single CO₂ trace corresponding to the CO₂ sensor input representing the highest detected CO₂ values from the plurality of CO₂ sensors.

In some embodiments, the first respiratory apparatus may include one or more of: a CO2 absorber configured to treat returned expired gas before recirculating to the patient in the first mode, a pressure limiting valve configured to maintain substantially stable pressure in the system in the first mode, a variable volume for displacement of gases in the first mode, a fresh gas flow for replenishing anaesthetic gas delivered to the patient in the first mode and a vaporizer for vaporizing volatile anaesthetic agents into breathing gas delivered to the patient in the first mode.

In some embodiments, the second respiratory apparatus may include one or more of: a flow source configured to generate gas flows through the system, and a humidifier configured to condition the breathing gas to a pre-determined temperature and/or humidity before delivery to the patient in the second mode.

Viewed from another aspect the present disclosure provides a system for delivering breathing gases to a patient, the system including: (a) a flow source configured to provide gas flows through the system in a gas delivery circuit; and (b) a gas delivery conduit circuit including an inspiratory gas flow path and an expiratory gas flow path, the system configured to switch between a first mode and a second mode. In the first mode, the system is operable to deliver the breathing gases to the patient via the inspiratory gas flow path and a first patient interface in fluid communication with the inspiratory flow path, and to deliver expiratory gases from the patient via the expiratory gas flow path and a second patient interface in fluid communication with the expiratory gas flow path, the breathing gases including a first flow parameter. In the second mode, the system is operable to deliver the breathing gases to the patient via the inspiratory gas flow path and a third patient interface, the breathing gases including a second flow parameter.

In some embodiments, the first flow parameter is different from the second flow parameter. The first flow parameter may include a first flow rate and the second flow parameter may include a second flow rate, wherein the first flow rate is less than the second flow rate. In some embodiments, the first flow rate is less than 15 L/min and the second flow rate is greater than 15 L/min. In some embodiments, the second flow rate is in the range of between about 20 L/min and between about 90 L/min, optionally about 40 L/min and about 70 L/min.

The first patient interface may include a non-sealing patient interface, such as e.g. a nasal cannula, and the second patient interface may include a sealing patient interface, such as e.g. a mask. The third patient interface may include a non-sealing patient interface, such as e.g. a nasal cannula. In some embodiments, the first and third patient interfaces are the same.

In some embodiments, the expiratory flow path is inoperable in the second mode.

In some embodiments, the system is in the first mode when the first and second patient interfaces are applied simultaneously to the patient and is in the second mode when only the third patient interface is applied to the patient. The first and third patient interfaces may include a non-sealing nasal cannula and the second patient interface may include a sealing mask, and the system may be in the first mode when the nasal cannula and mask are applied to the patient and the system may be in the second mode when only the nasal cannula is applied to the patient. In some embodiments, the mask is configured to seal over the nasal cannula and with the patient.

In some embodiments, the system includes a third mode and a fourth patient interface and the system may be configured to deliver the breathing gases to the patient via the inspiratory gas flow path and the fourth patient interface and to deliver expiratory gases from the patient via the expiratory gas flow path and the fourth patient interface. The breathing gases may include a third flow parameter. The fourth patient interface may include a sealing patient interface, such as e.g. an invasive patient interface, laryngeal mask airway or endotracheal tube.

In some embodiments, the first flow parameter includes a pressure and/or volume parameter.

In some embodiments, the third flow parameter include one or more of a flow rate, pressure or volume parameter.

In some embodiments, the system may be configured to control the delivery of breathing gases to the patient based on pressure and/or volume.

In some embodiments, system includes an inspiratory conduit defining at least a portion of the inspiratory gas flow path and an expiratory conduit defining at least a portion of the expiratory gas flow patient, and a common connector provided at an end of the inspiratory and expiratory conduits, the common connector configured to connect to the one or more patient interfaces.

In some embodiments, the system includes a controller in communication with the flow source, and one or more of a sensor or an input interface in communication with the controller to provide an input to the controller to control the flow source to provide flow of breathing gases in the first or second mode. The sensor and/or input interface may be configured to provide an input to the controller to control the flow source to provide flow of breathing gases in the third mode.

In some embodiments, the system includes a humidifier, wherein the breathing gases are heated and humidified by the humidifier in the second mode prior to being delivered to the patient.

In some embodiments, the expiratory gases from the patient are returned to the inspiratory gas flow path in the first mode. In some embodiments, the expiratory gases from the patient are returned to the inspiratory gas flow path in the third mode. In some embodiments, the system including a CO₂ remover configured to remove CO₂ from the expiratory gases before returning the expiratory gases to the inspiratory gas flow path.

When a high flow system and anaesthesia machine are integrated, it may be desirable to have a configuration that allows for easy and sufficient humidification of breathing gases that are delivered during high flow respiratory support. It may also be desirable to enable humidification of the breathing gases in the high flow mode.

Viewed from another aspect, the present disclosure provides a respiratory apparatus for delivering breathing gases to a patient, the respiratory apparatus including: a flow source for providing a flow of breathing gases in an inspiratory flow path for delivery to the patient; a mount for coupling with at least one vaporizer for vaporizing one or more volatile anaesthetic agents into the flow of breathing gases in the inspiratory flow path before delivery to the patient; and a return path for recirculating expired gases received from the patient via an expiratory flow path to the inspiratory flow path; wherein the mount is couplable with a humidification component for conditioning the flow of breathing gases in the inspiratory flow path to a pre-determined temperature and/or humidity before delivery to the patient.

In some embodiments, operation of the humidification component prevents delivery of the one or more volatile anaesthetic agents into the flow of breathing gases in the inspiratory flow path. Operation of the humidification component may disable operation of the at least one vaporizer.

In some embodiments, the respiratory apparatus further includes an interlocking mechanism to prevent simultaneous operation of the humidification component and the at least one vaporizer. The interlocking mechanism may be configured to enable operation of the humidification component or the at least one vaporizer when in an unlocked configuration, and disable operation of the humidification component or the at least one vaporizer when in a locked configuration.

The humidification component and the at least one vaporizer may be configured for cooperation with one another to provide the interlocking mechanism. The humidification component and the at least one vaporizer may be mountable adjacent to one another on the respiratory apparatus. For example, the mount may include a plurality of slots for coupling with the humidification component and the at least one vaporizer in a side-by-side arrangement. The slots may be configured to receive a housing of the humidification component and a housing of the at least one vaporizer. The mount may be configured to receive the housing of the humidification component and the housing of the at least one vaporizer through sliding engagement with the slots.

In some embodiments, the humidification component and the at least one vaporizer may each include a locking element associated with a housing thereof. The locking element may be configured to engage with a corresponding locking element associated with a housing of the other of the humidification component and the at least one vaporizer to provide the interlocking mechanism. The locking element may include at least one locking pin which is retractable within the housing in the locked configuration and extendable from the housing in the unlocked configuration. The locking element may include two or more locking pins, where each locking pin is independently retractable within the housing in the locked configuration and extendable from the housing in the unlocked configuration.

In some embodiments, the humidification component and the at least one vaporizer include a slot associated with a housing thereof, and at least one locking pin is slidably movable between the slots of the humidification component and the at least one vaporizer to provide the interlocking mechanism. The at least one locking pin may be positionable with the slot of the humidification component or the at least one vaporizer in the locked configuration, and may be positionable within the slot of the other of the humidification component or the at least one vaporizer in the unlocked configuration.

In some embodiments, the respiratory apparatus further includes a switching mechanism configured to enable selective operation of the humidification component and the at least one vaporizer. Upon activation of the switching mechanism to operate the humidification component or the at least one vaporizer, the other of the humidification component and the at least one vaporizer may be prevented from operating until the switching mechanism is deactivated.

The switching mechanism may include each of the at least one vaporizer and the humidification component being operable by a switch, and the switches may be linked together to prevent simultaneous operation of the humidification component and the at least one vaporizer.

In some embodiments, the switching mechanism is coupled with the interlocking mechanism. Upon activation of the switching mechanism to operate the humidification component or the at least one vaporizer, the interlocking mechanism may enable operation of the humidification component or the at least one vaporizer and may disable operation of the other of the humidification component or the at least one vaporizer.

In some embodiments, the humidification component includes a humidification chamber through which breathing gases are received and conditioned to the pre-determined temperature and/or humidity. The humidification chamber may be configured to be coupled with the mount. A housing of the humidification chamber may be configured to be slidably received onto the mount. For example, the mount may include a plurality of slots and the housing of the humidification chamber may be slidably receive into one of the slots.

The mount may include a heating element for heating liquid in the humidification chamber. The humidification chamber may include a conductive plate for conducting heat from the heating element in the mount. In other embodiments, the humidification chamber includes a heating element for heating liquid in the humidification chamber. The humidification chamber may be configured to electrically connect with the respiratory apparatus for operation of the humidification chamber.

In some embodiments, the humidification component includes a humidifier having a humidification chamber couplable with a humidification base unit for operation of the humidification chamber. The humidification base unit may be configured to be coupled with the mount. A housing of the humidification base unit may be configured to be slidably received onto the mount. For example, the mount may include a plurality of slots and the housing of the humidification base unit may be slidably receive into one of the slots. The humidification base unit may include a heating element for heating liquid in the humidification chamber.

The humidification chamber as disclosed herein may include an inlet port for receiving a flow of the breathing gases from the respiratory apparatus, and an outlet port for delivering the conditioned flow of breathing gases to the patient. The outlet port may be couplable with an inspiratory conduit for delivering the conditioned flow of breathing gases to the patient via a patient interface.

In other embodiments, the humidification chamber as disclosed herein may include an inlet port for receiving a flow of the breathing gases from the respiratory apparatus, and a return port for returning the conditioned flow of breathing gases to the respiratory apparatus. The return port may be couplable with the respiratory apparatus for returning the conditioned flow of breathing gases.

In some embodiments, the humidification chamber includes a liquid inlet that connects to a liquid reservoir for refilling of the humidification chamber. The humidification chamber may include a flow control mechanism to control flow of liquid into the humidification chamber. The humidification chamber may include at least one sensor for detecting a level of liquid in the humidification chamber. The humidification chamber may include a float valve for controlling a level of liquid in the humidification chamber.

In some embodiments, the humidification component is couplable with the mount via an adapter. The adapter may include a first inlet port for receiving a flow of the breathing gases from the respiratory apparatus, and a first outlet port for delivering the flow of breathing gases to the humidification component. The adapter may further include a second inlet port for receiving a conditioned flow of breathing gases from the humidification component. The adapter may also include a second outlet port for delivering the conditioned flow of breathing gases from the humidification component to the respiratory apparatus.

In some embodiments, the adapter is configured to electrically connect with the respiratory apparatus for operation of the humidification component. The adapter may include a first power connector to provide an electrical connection with the respiratory apparatus. The adapter may also include a second power connector to provide an electrical connection with the humidification component.

The respiratory apparatus may be configured to operate in the following modes of operation: a first mode in which the respiratory apparatus delivers breathing gases to the inspiratory flow path and receives return of expired gases via the expiratory flow path; and a second mode in which the respiratory apparatus delivers breathing gases to the inspiratory flow path at a pre-determined flow rate without return of expired gases from the patient.

In some embodiments, the respiratory apparatus is further configured to detect coupling of the humidification component with the mount to enable operation of the respiratory apparatus in the second mode. For example, the mount may include a sensor for detecting coupling of the humidification component. In the second mode, the humidification component may be operable to condition the flow of breathing gases in the inspiratory flow path to the pre-determined temperature and/or humidity before delivery to the patient.

In some embodiments, the respiratory apparatus further includes a CO₂ absorber configured to treat returned expired gas from the patient before recirculating to the patient in the first mode. In the second mode, the CO₂ absorber may be further configured to condition the breathing gases in the inspiratory flow path to a pre-determined temperature and/or humidity before delivery to the patient. In the second mode, the CO₂ absorber may be configured to condition the breathing gases to the pre-determined temperature and/humidity by one or both of changing an amount of soda lime present in the CO2 absorber, and changing an amount of CO2 provided to the soda lime present in the CO2 absorber.

In the first mode, the respiratory apparatus may be operable to deliver breathing gases to the patient by a first patient interface forming a sealing interface with the patient's airway and returning expired gases to the respiratory apparatus via the expiratory flow path. In the first mode, the breathing gases include one or more anaesthetic agents. The first patient interface may be a mask or endotracheal tube.

In the second mode, the respiratory apparatus may be operable to deliver breathing gases to the patient by a second patient interface forming a non-sealing interface with the patient's airway. The second patient interface may be a nasal cannula.

In the second mode, the pre-determined flow rate may be in a range of about 20 L/min to about 90 L/min.

In the second mode, the pre-determined flow rate may be in a range of about 40 L/min to about 70 L/min.

In some embodiments, the respiratory apparatus is configurable in gas flow communication with a gas delivery apparatus receiving a supply of gas including NO, O2 and air. The gas delivery apparatus may include a gas mixing element for combining one or more of NO, O2 and air in a proportion required for delivering the breathing gases in the first mode and/or the second mode, and a gas outlet supplying gas from the gas delivery apparatus to the respiratory apparatus.

In some embodiments, the respiratory apparatus is configurable in gas flow communication with a flow meter for controlling the flow of gas including one or both of air and O2 in the second mode.

In some embodiments the respiratory apparatus is configurable in gas flow communication with one or more of: a pressure limiting valve configured to maintain substantially stable pressure in the respiratory apparatus in the first mode; a variable volume for displacement of gases in the first mode; a fresh gas flow for replenishing breathing gases delivered to the patient in the first mode; and a vaporizer for vaporizing one or more volatile anaesthetic agents into the flow of the breathing gases before delivery to the patient in the first mode.

In another aspect, the present disclosure provides a humidification component for use with a respiratory apparatus for delivering breathing gases to a patient, the respiratory apparatus including: a flow source for providing a flow of breathing gases in an inspiratory flow path for delivery to the patient; a mount for coupling with at least one vaporizer for vaporizing one or more volatile anaesthetic agents into the flow of breathing gases in the inspiratory flow path before delivery to the patient; and a return path for recirculating expired gases received from the patient via an expiratory flow path to the inspiratory flow path; wherein the humidification component is couplable with the mount for conditioning the flow of breathing gases in the inspiratory flow path to a pre-determined temperature and/or humidity before delivery to the patient.

In some embodiments, operation of the humidification component prevents delivery of the one or more volatile anaesthetic agents into the flow of breathing gases in the inspiratory flow path. Operation of the humidification component may disable operation of the at least one vaporizer.

In some embodiments, the humidification component is couplable with the mount to provide an interlocking mechanism to prevent simultaneous operation of the humidification component and the at least one vaporizer. The interlocking mechanism may be configured to enable operation of the humidification component or the at least one vaporizer when in an unlocked configuration, and disable operation of the humidification component or the at least one vaporizer when in a locked configuration.

The humidification component may be couplable with the mount to enable cooperation with the at least one vaporizer to provide the interlocking mechanism. The humidification component may be mountable on the respiratory apparatus adjacent to the at least one vaporizer. For example, the mount may include a plurality of slots for coupling with the humidification component and the at least one vaporizer in a side-by-side arrangement. The slots may be configured to receive a housing of the humidification component and a housing of the at least one vaporizer. The mount may be configured to receive the housing of the humidification component and the housing of the at least one vaporizer through sliding engagement with the slots.

In some embodiments, the humidification component includes a housing having a locking element configured to engage with a corresponding locking element associated with a housing of the at least one vaporizer to provide the interlocking mechanism. The locking element may include at least one locking pin which is retractable within the housing in the locked configuration and extendable from the housing in the unlocked configuration. The locking element may include two or more locking pins, where each locking pin is independently retractable within the housing in the locked configuration and extendable from the housing in the unlocked configuration.

In some embodiments, the humidification component includes a housing having a slot, and at least one locking pin is slidably movable between the slot of the humidification component and a slot associated with a housing of the at least one vaporizer to provide the interlocking mechanism. The at least one locking pin may be positionable within the slot of the humidification component in the locked configuration and may be positionable within the slot of the at least one vaporizer in the unlocked configuration.

In some embodiments, the respiratory apparatus further includes a switching mechanism configured to enable selective operation of the humidification component and the at least one vaporizer. Upon activation of the switching mechanism to operate the humidification component, the at least one vaporizer may be prevented from operating until the switching mechanism is deactivated.

The switching mechanism may include the humidification component being operable by a switch, and the switch may be linked together with a switch of the at least one vaporizer to prevent simultaneous operation of the humidification component and the at least one vaporizer.

The humidification component may further include a switch operable by a user to enable selective operation of the humidifier. For example, the switch may be a manually operated switch, such as a button or dial, on a housing of the humidification component.

In some embodiments, the switching mechanism is coupled with the interlocking mechanism. Upon activation of the switching mechanism to operate the humidification component, the interlocking mechanism may enable operation of the humidification component and disable operation of the at least one vaporizer.

In some embodiments, the humidification component includes a humidification chamber through which breathing gases are received and conditioned to the pre-determined temperature and/or humidity. The humidification chamber may be configured to be coupled with the mount. A housing of the humidification chamber may be configured to be slidably received onto the mount. For example, the mount may include a plurality of slots and the housing of the humidification chamber may be slidably received into one of the slots.

The mount may include a heating element for heating liquid in the humidification chamber. The humidification chamber may include a conductive plate for conducting heat from the heating element in the mount. In other embodiments, the humidification chamber includes a heating element for heating liquid in the humidification chamber. The humidification chamber may be configured to electrically connect with the respiratory apparatus for operation of the humidification chamber.

In some embodiments, the humidification component includes a humidifier having a humidification chamber couplable with a humidification base unit for operation of the humidification chamber. The humidification base unit may be configured to be coupled with the mount. A housing of the humidification base unit may be configured to be slidably received onto the mount. For example, the mount may include a plurality of slots and the housing of the humidification base unit may be slidably received into one of the slots. The humidification base unit may include a heating element for heating liquid in the humidification chamber.

The humidification chamber as disclosed herein may include an inlet port for receiving a flow of the breathing gases from the respiratory apparatus, and an outlet port for delivering the conditioned flow of breathing gases to the patient. The outlet port may be couplable with an inspiratory conduit for delivering the conditioned flow of breathing gases to the patient via a patient interface.

In other embodiments, the humidification chamber as disclosed herein may include an inlet port for receiving a flow of the breathing gases from the respiratory apparatus, and a return port for returning the conditioned flow of breathing gases to the respiratory apparatus. The return port may be couplable with the respiratory apparatus for returning the conditioned flow of breathing gases.

In some embodiments, the humidification chamber includes a liquid inlet that connects to a liquid reservoir for refilling of the humidification chamber. The humidification chamber may include a flow control mechanism to control flow of liquid into the humidification chamber. The humidification chamber may include at least one sensor for detecting a level of liquid in the humidification chamber. The humidification chamber may include a float valve for controlling a level of liquid in the humidification chamber.

In some embodiments, the humidification component is couplable with the mount via an adapter. The adapter may include a first inlet port for receiving a flow of the breathing gases from the respiratory apparatus, and a first outlet port for delivering the flow of breathing gases to the humidification component. The adapter may further include a second inlet port for receiving a conditioned flow of breathing gases from the humidification component. The adapter may also include a second outlet port for delivering the conditioned flow of breathing gases from the humidification component to the patient or to the respiratory apparatus.

The respiratory apparatus may be configured to operate in the following modes of operation: a first mode in which the respiratory apparatus delivers breathing gases to the inspiratory flow path and receives return of expired gases via the expiratory flow path; and a second mode in which the respiratory apparatus delivers breathing gases to the inspiratory flow path at a pre-determined flow rate without return of expired gases from the patient.

In some embodiments, the respiratory apparatus is further configured to detect coupling of the humidification component with the mount to enable operation of the respiratory apparatus in the second mode. For example, the mount may include a sensor for detecting coupling of the humidification component. In the second mode, the humidification component may be operable to condition the flow of breathing gases in the inspiratory flow path to the pre-determined temperature and/or humidity before delivery to the patient.

In some embodiments, the respiratory apparatus further includes a CO2 absorber configured to treat returned expired gas from the patient before recirculating to the patient in the first mode. In the second mode, the CO2 absorber may be further configured to condition the breathing gases in the inspiratory flow path to a pre-determined temperature and/or humidity before delivery to the patient. The respiratory apparatus may be further configured to enable the CO2 absorber in the second mode to condition the breathing gases to the pre-determined temperature and/humidity by one or both of changing an amount of soda lime present in the CO2 absorber, and changing an amount of CO2 provided to the soda lime present in the CO₂ absorber.

In the first mode, the respiratory apparatus may be operable to deliver breathing gases to the patient by a first patient interface forming a sealing interface with the patient's airway and returning expired gases to the respiratory apparatus via the expiratory flow path. In the first mode, the breathing gases include one or more anaesthetic agents. The first patient interface may be a mask or endotracheal tube.

In the second mode, the respiratory apparatus may be operable to deliver breathing gases to the patient by a second patient interface forming a non-sealing interface with the patient's airway. The second patient interface may be a nasal cannula.

In the second mode, the pre-determined flow rate may be in a range of about 20 L/min to about 90 L/min.

In the second mode, the pre-determined flow rate may be in a range of about 40 L/min to about 70 L/min.

In some embodiments, the respiratory apparatus is configurable in gas flow communication with a gas delivery apparatus receiving a supply of gas including NO, O2 and air. The gas delivery apparatus may include a gas mixing element for combining one or more of NO, O2 and air in a proportion required for delivering the breathing gases in the first mode and/or the second mode, and a gas outlet supplying gas from the gas delivery apparatus to the respiratory apparatus.

In some embodiments, the respiratory apparatus is configurable in gas flow communication with a flow meter for controlling the flow of gas including one or both of air and O2 in the second mode.

In some embodiments the respiratory apparatus is configurable in gas flow communication with one or more of: a pressure limiting valve configured to maintain substantially stable pressure in the respiratory apparatus in the first mode; a variable volume for displacement of gases in the first mode; a fresh gas flow for replenishing breathing gases delivered to the patient in the first mode; and a vaporizer for vaporizing one or more volatile anaesthetic agents into the flow of the breathing gases before delivery to the patient in the first mode.

### BRIEF DESCRIPTION OF DRAWINGS

The invention will now be described in greater detail with reference to the accompanying drawings in which like features are represented by like numerals. It is to be understood that the embodiments shown are examples only and are not to be taken as limiting the scope of the invention as defined in the claims appended hereto.

FIG. 1A is a schematic diagram showing components of a prior art anaesthesia machine. FIG. 1B is a schematic diagram of a prior art ventilator 20.

FIG. 2 is a schematic diagram of components of a prior art high flow system.

FIG. 3 is a schematic illustration showing components in a system for delivering breathing gases to a patient according to an embodiment of the disclosure.

FIGS. 4A and 4B are schematic illustrations of a gas delivery apparatus for switching gas flows according to an embodiment of the disclosure.

FIGS. 5A and 5B are schematic illustrations of a gas delivery apparatus for switching gas flows according to another embodiment of the disclosure, having a common gas outlet.

FIGS. 6A and 6B are schematic illustrations showing a gas delivery apparatus and second switching element for switching gas flows according to another embodiment of the disclosure.

FIGS. 7A and 7B are schematic illustrations of a gas diverter for switching gas flows according to another embodiment of the disclosure.

FIG. 8 is a schematic illustration of a switching mechanism located between a gas source and the first and second respiratory apparatuses according to another embodiment of the disclosure.

FIG. 9 is a schematic illustration of a switching mechanism including a flow rate selector according to an embodiment of the disclosure.

FIG. 10 is a schematic illustration of a switching mechanism including a flow rate selector according to another embodiment of the disclosure.

FIG. 11 is a schematic illustration of a switching mechanism including a flow rate selector with pressure controlled actuator.

FIG. 12A is a schematic illustration of a 3-way actuator which provides coupling a ventilation bag in a manual first mode or bellows in a mechanical first mode. FIG. 12B is a schematic illustration of a 3 way actuator with a pressure controlled actuator.

FIGS. 13A and 13B are schematic illustrations showing how a 3 position switch could provide physical fluid couplings required to provide for switching between modes.

FIG. 14 is a schematic illustration of a respiratory apparatus 1000 operable to deliver breathing gases to a patient in three modes including anesthetic ventilation, high flow and flush modes.

FIGS. 15A and 15B are schematic drawings of a system for delivering different modes of respiratory support including ventilation, anaesthesia and high flow respiratory support.

FIGS. 16A to 16E are schematic illustrations showing various alternative embodiments for the system of FIGS. 15A and 15B.

FIG. 17 is a schematic illustration of a modular system for delivering different modes of respiratory support including ventilation, anaesthesia and high flow respiratory support.

FIG. 18A is an illustration of a gas flow switching mechanism according to an embodiment of the disclosure. FIG. 18B shows the switching mechanism in a top sectional view in a first mode and FIG. 18C shows the switching mechanism in a top sectional view in a second mode.

FIGS. 19A and 19B illustrate a gas flow switching mechanism according to another embodiment of the disclosure, in first and second modes respectively.

FIG. 20 is a schematic illustration of a system attached to a multi-lumen assembly for delivery of breathing gases.

FIG. 21 is a schematic illustration showing a patient end of a multi-lumen assembly having a connector portion.

FIGS. 22A to 22C are schematic illustrations showing retaining mechanisms according to various embodiments of the disclosure.

FIGS. 23A and 23B are schematic illustrations showing an actuator for switching flow of breathing gases toward a patient end of the assembly.

FIG. 24 is a schematic illustration of a patient end connector for use with a multi-lumen assembly to selectively control a flow of breathing gases toward a patient end of the assembly.

FIGS. 25 and 26 illustrate patient interfaces with CO₂ sensing according to embodiments of the disclosure.

FIGS. 27 and 28 are schematic illustrations showing a piston driven assembly to selectively direct expired gases from a patient mask and nasal cannula to a gas sampling line, with the piston in a first position and second position respectively.

FIGS. 29A and 29B are schematic illustrations showing use of a pressure controlled flow diverter to selectively direct expired gases from a patient mask (FIG. 29A) and nasal cannula (FIG. 29B) to a gas sampling line.

FIG. 30 is a schematic illustration of a three way switch for selectively directing expiratory gas from a nasal cannula, endotracheal tube or a sealing mask into a gas sampling line.

FIGS. 31 to 33 illustrate use of multiple patient interfaces in the delivery of respiratory support according to embodiments of the present disclosure.

FIG. 34 is a schematic illustration of a connector that may be used to facilitate interchanging of components for delivery of different modes of respiratory support according to embodiments of the present disclosure.

FIG. 35 is a schematic illustration of another connector for use according to embodiments of the present disclosure.

FIG. 36 is a schematic illustration of another connector for use with a nasal cannula used to deliver different modes of respiratory support according to embodiments of the present disclosure.

FIGS. 37A and 37B are schematic illustrations of a connector which is a variation on the connector of FIG. 36.

FIG. 38 is a schematic diagram of a respiratory apparatus for delivering breathing gases to a patient, couplable with at least one vaporizer and a humidification component, according to some embodiments of the disclosure.

FIG. 39 is a front view of the respiratory apparatus of FIG. 38, depicted as an anaesthesia machine, including a vaporizer and humidification component coupled with a mount of the anaesthesia machine, according to some embodiments of the disclosure.

FIG. 40 is an enlarged view of the mount of the anaesthesia machine shown in FIG. 39 with the humidification component removed and including a heating element in the mount, according to some embodiments of the disclosure.

FIG. 41 is a sectional view of a humidification component for use with a respiratory apparatus for delivering breathing gases to a patient, where the humidification component includes a humidification chamber having a heating element electrically connected with the mount, and the humidification chamber returns conditioned breathing gases to the respiratory apparatus, according to some embodiments of the disclosure.

FIG. 42 is a sectional view of another humidification component for use with a respiratory apparatus for delivering breathing gases to a patient, where the humidification component includes a humidifier having a humidification chamber and a humidification base unit with a heating element electrically connected with the mount, and the humidification chamber receives breathing gases from the respiratory apparatus and delivers conditioned breathing gases to the patient via an inspiratory conduit, according to some embodiments of the disclosure.

FIG. 43 is a sectional view of another humidification component for use with a respiratory apparatus for delivering breathing gases to a patient, where the humidification component includes a humidification chamber having a conductive plate coupled with a heating element in the mount, and the humidification chamber returns conditioned breathing gases to the respiratory apparatus, according to some embodiments of the disclosure.

FIG. 44 is a sectional view of another humidification component for use with a respiratory apparatus for delivering breathing gases to a patient, where the humidification component includes a humidifier having a humidification chamber and a humidification base unit with a heating element, and the humidification chamber returns conditioned breathing gases to the respiratory apparatus, according to some embodiments of the disclosure.

FIG. 45 is a schematic diagram of an adapter for coupling a humidification component with a respiratory apparatus for delivering breathing gases to a patient, where the adapter is couplable with the mount of the respiratory apparatus, according to some embodiments of the disclosure.

FIG. 46 is a schematic diagram of a vaporizer with an interlocking mechanism associated with the housing, the housing including two locking pins which are coupled with a switch on a dial, according to some embodiments of the disclosure.

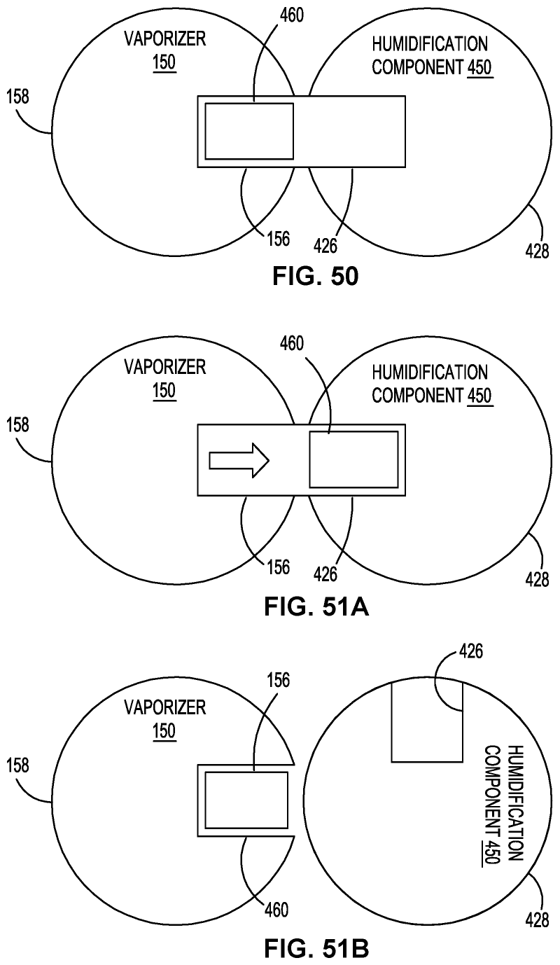

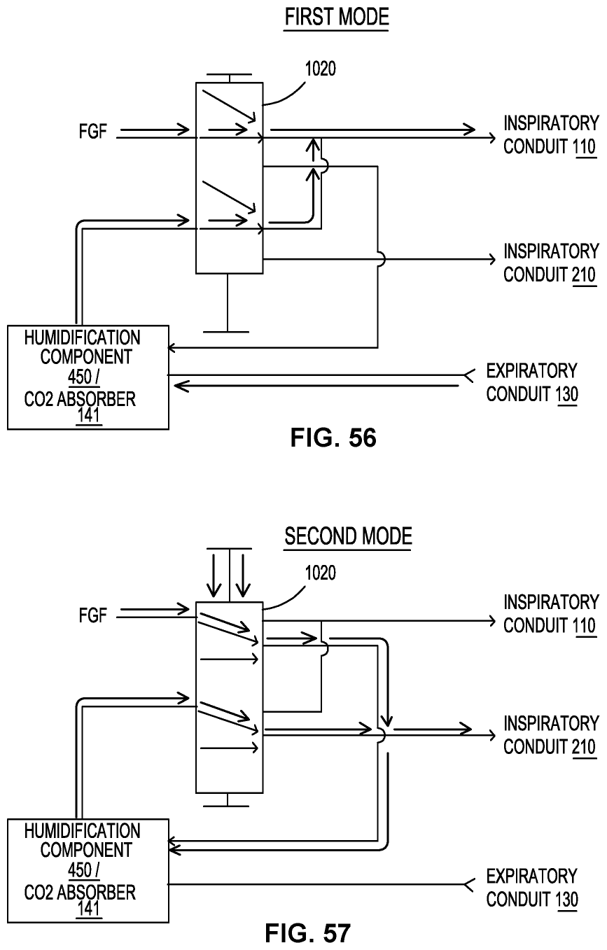

FIGS. 43A-C are schematic diagrams showing the vaporizer of FIG. 46 switched on with the locking pins extended (FIG. 47A), switched off with the locking pins retracted (FIG. 47B), and in a locked state with one locking pin fully retracted into the housing (FIG. 47C), according to some embodiments of the disclosure.

FIG. 48 is a schematic diagram showing the vaporizer of FIG. 46 positioned adjacent to a humidification component having the same interlocking mechanism associated with the housing and a switch on a dial, according to some embodiments of the disclosure.

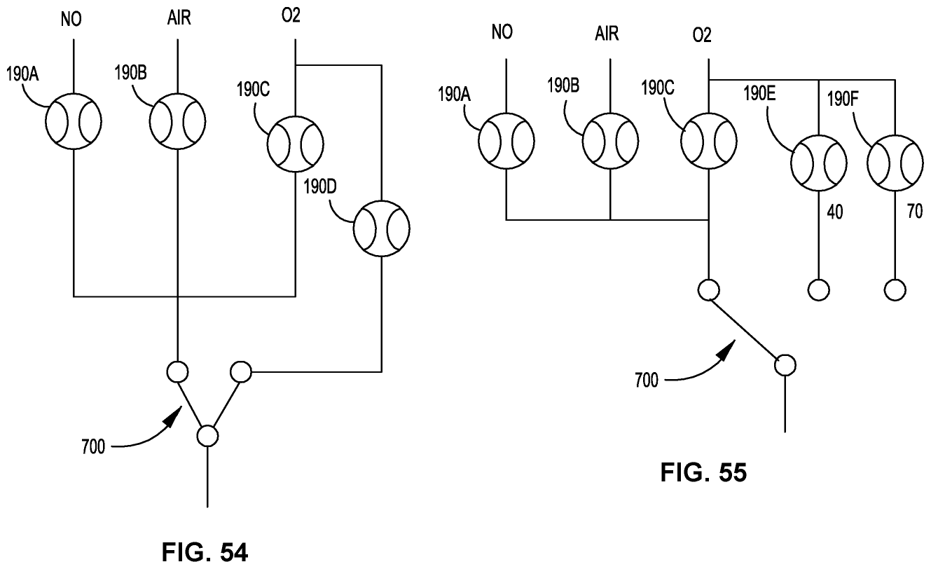

FIGS. 45A-B are schematic diagrams showing the vaporizer and humidification component with the interlocking mechanism of FIG. 48, with the vaporizer switched on with the locking pins extended, thereby locking the humidification component in the OFF position (FIG. 49A), and with the humidification component switched on with locking pins extended, thereby locking the vaporizer in the OFF position (FIG. 49B), according to some embodiments of the disclosure.

FIG. 50 is a schematic diagram showing another interlocking mechanism in which the vaporizer and humidification component include a slot in the housing and a locking pin can slide between the slots to lock either the vaporizer or humidification component in the OFF position, according to some embodiments of the disclosure.

FIGS. 47A-B are schematic diagrams showing the interlocking mechanism of FIG. 50 in which the locking pin is slid over to lock the humidification control in the OFF position (FIG. 51A) and in which the humidification component is switched on, and the vaporizer control is locked in the OFF position (FIG. 51B), according to some embodiments of the disclosure.

FIG. 52 is a schematic diagram showing switching between a vaporizer and humidification component in the respiratory apparatus, according to some embodiments of the disclosure.

FIG. 53 is a schematic diagram showing mechanically interlocked switches for a vaporizer and humidification component in the respiratory apparatus, according to some embodiments of the disclosure.

FIG. 54 is a schematic diagram showing a fixed flow meter for a second mode of operating the respiratory apparatus, being a high flow mode, according to some embodiments of the disclosure.

FIG. 55 is a schematic diagram showing two variable flow meters for a second mode of operating the respiratory apparatus, being a high flow mode, according to some embodiments of the disclosure.

FIG. 56 is a schematic diagram showing gas flow through a respiratory apparatus in a first mode being an anaesthetic ventilation mode, according to some embodiments of the disclosure.

FIG. 57 is a schematic diagram showing gas flow through a respiratory apparatus in a second mode being a high flow mode, according to some embodiments of the disclosure.

### DETAILED DESCRIPTION

Embodiments of the invention are discussed herein by reference to the drawings which are not to scale and are intended merely to assist with explanation of the invention.

### Components of Anaesthesia Machines

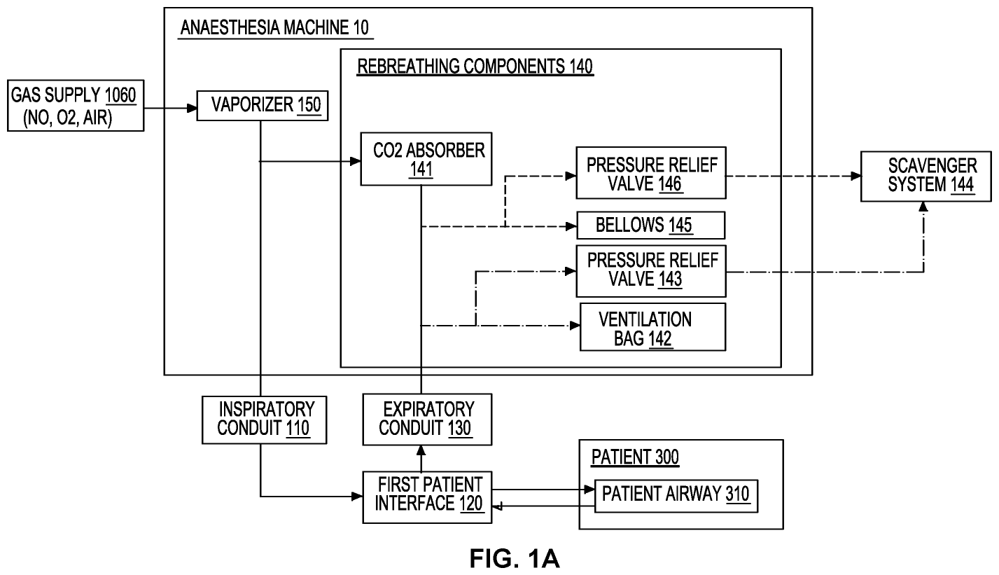

FIG. 1A is a schematic diagram showing components of an anaesthesia machine 10, which is configurable to receive a gas supply 1060 for delivering a respiratory support to a patient 300 through piped connections known in the art. The gas supply 1060 may include one or more of an anaesthetic gas (e.g. nitric oxide (NO)), oxygen (O₂) and air supply. The air supply may be ambient air. Flow meters may be incorporated into the gas supply 1060, or placed upstream of the anaesthesia machine 10, or incorporated into it, to control the flow of gases through the machine. Typically such flow meters are manually controlled but may also be precision controlled by a controller of the anaesthesia machine.

A breathing circuit delivers gases to the patient 300 and returns expired gases to rebreathing components 140. Typically the breathing circuit includes corrugated tubing, valves and one or more patient interfaces for directing gases into the patient's airway and removing expired gases. In the schematic illustration of FIG. 1A the breathing circuit is simplified and denoted as including (but not limited to) inspiratory conduit 110 and first patient interface 120 which directs gas into the airway 310 of patient 300 and expiratory conduit 130 which collects expired gases. Thus, first patient interface 120 may be a sealing interface such as a sealing mask or endotracheal tube and may be configured to direct expired gases from the patient 300 to an expiratory flow path 130 which returns the expired gases to rebreathing components 140 of the anaesthesia machine 10. The inspiratory and expiratory conduits are typically connected to the patient interface by a wye piece connector.

One or more vaporizers 150 convert volatile anaesthetics such as isoflurane and sevoflurane from liquid to vapour, and control introduction of these agents into the breathing circuit in accurately controlled concentration and dosages as required by the user, typically an anaesthetist clinician. Typically, vaporizers 150 are manually controlled but may be precision controlled by a controller of the respiratory apparatus. In some embodiments, vaporizers 150 feed into the rebreathing components 140.

Integrated into the anaesthesia machine 10 is a ventilation system which ventilates patient 300 during induction and after administration of anaesthetic agents to achieve ongoing anaesthesia. A manual ventilation bag 142 is typically used during induction (dot-dash lines inside rebreathing components 140) when volatiles are being delivered and prior to the patient being intubated. The compliance of the ventilation bag 142 enables the patient to breathe in and out a fixed volume of gas through a sealing first patient interface 120 in the form of a face mask. Once intubated, the ventilation mode changes from manual to mechanical, effectively isolating the manual ventilation bag 142 and associated pressure relief valve 143 from the rebreathing components 140 so that ventilation occurs via a mechanical system (dash lines inside rebreathing components 140). Typically this involves a collapsible bellows 145 that controls the tidal volume and timing of breaths delivered to the patient through a sealing first patient interface 120 in the form of an endotracheal tube. Gases provided to the patient may be pressure, flow or volume controlled. Pressure relief valves 143, 146 provide for release of excess gases from the rebreathing components 140 (arising from fresh gas flow from vaporizers 150 and returned expired patient gases) while preventing ambient air from entering the breathing circuit.

Rebreathing components 140 provide a gas recirculation system in which exhaled gases from the patient are treated as they flow around the circuit and are then re-inhaled. This provides advantages by reusing oxygen and volatiles that are present in the expired gas flow from the patient, reducing costs as well as the presence of anaesthetic agents in the atmosphere. Expired gases in the rebreathing components 140 are passed through CO₂ absorber 141 which may include a canister containing soda lime (or another CO₂ absorbing substance). The soda lime (a mixture of NaOH & Ca(OH)2) acts as a CO₂ scrubber to remove CO₂ before gases in the rebreathing components 140 re-enter the inspiratory conduit 110. Additionally, gases from pressure relief valves 143, 146 are directed via an exhaust (not shown) to an external scavenger system 144 which filters and collects anaesthetic gases from the gas flow.

It is to be understood that further features may be provided as part of an anaesthesia machine 10, such as e.g. patient monitoring, suction, pressure gauges, regulators and “pop-off” valves to protect the patient and components of the machine from high pressure gases, as are known in the art. For simplicity, these are not included in the example shown.

FIG. 1B is a schematic diagram of a ventilator 20 which may be used in an Intensive Care Unit (ICU). Ventilator 20 ventilates a patient with gases from gas supply 1060 and often with active humidification by humidifier 420 which is configured to heat and humidify gases delivered to the patient's airway 310. A ventilator 20 can support the patient's own breathing or replace it by delivering respiratory gases to a patient that are controlled to replicate ‘normal’ inhalation and exhalation breathing phases. Mechanical ventilator 184 may include a flow modulator and/or blower, and controls the pressure, volume and breathing rate of breathing gases delivered through inspiratory conduit 110 which is delivered to the patient by a sealing first patient interface 120. The sealing first patient interface may be invasive (e.g. endotracheal tube or laryngeal mask airway (LMA)) or non-invasive (e.g. sealing face mask). Exhaled gases leave the patient via the first patient interface and expiratory conduit 130 where they are treated e.g. by filter 182 and released to atmosphere. In some non-invasive ventilation systems expired gases exit through a vent or exhaust ports in the patient interface 120 or the expiratory conduit which enables expired gases to exit to atmosphere without returning to the ventilator apparatus 20.

### Components of High Flow Systems

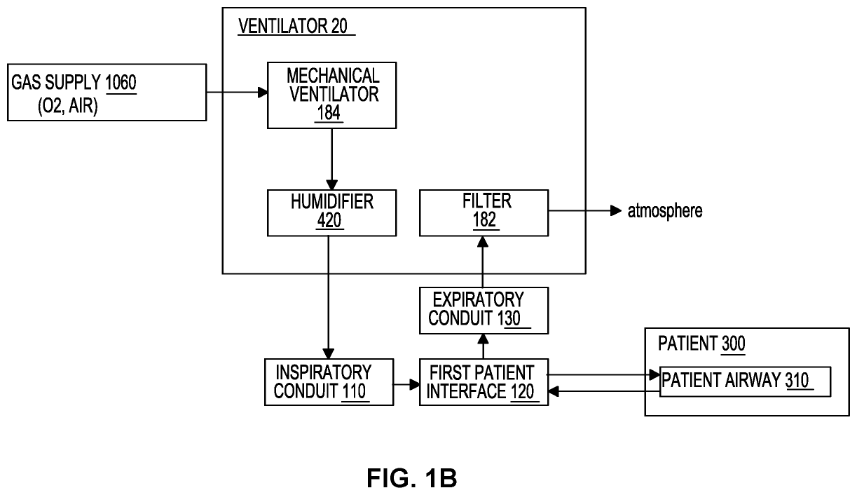

FIG. 2 is a schematic diagram of components of a high flow system 30 which is configurable to receive from a gas supply 1060 for delivering high flow respiratory support to a patient 300. The gas supply 1060 may be one or more of an anaesthetic gas (e.g. nitric oxide (NO)), oxygen (O2) or air supply, preferably an O2 and/or air supply. The air supply may be ambient air. High flow system 30 has a flow modulator 250 configured to generate gas flows that are passed through a humidifier 420 which is configured to heat and humidify gas flows generated by the flow modulator 250. In some embodiments, the flow modulator 250 may comprise a gas supply 1060 as described below. The humidified high gas flow is delivered to the patient 300 by a second inspiratory conduit 210 and a non-sealing second patient interface 220. Typically this is a nasal cannula, which directs the high flow of breathing gases into the patient's airway 310 through one or both nares. An optional filter 230 may be provided between the inspiratory conduit 210 and the second patient interface 220 so that components of the breathing circuit upstream from the filter can be reused without risk of contamination by any inadvertently captured expired gases by the second patient interface 220. In some configurations, the flow modulator 250 is configured to provide gases to the patient through high flow system 30. In some embodiments, the flow modulator comprises a gas generation means, for example a blower adapted to receive gases from the environment outside of the high flow system 30 and propel them through the high flow system 30. In some configurations, the flow modulator 250 may comprise a source available from a hospital gas outlet or wall supply (e.g. oxygen or air), or one or more containers of compressed air and/or another gas and one or more valve arrangements adapted to control the rate at which gases leave the one or more containers. In some configurations, the flow modulator 250 may comprise an oxygen concentrator.

In this specification, “high flow” means, without limitation, any gas flow with a flow rate that is higher than usual/normal, such as higher than the normal inspiration flow rate of a healthy patient, or higher than some other threshold flow rate that is relevant to the context. It can be provided by a non-sealing respiratory system with substantial leak, for example happening at the entrance of the patient's airways. It can also be provided with humidification to improve patient comfort, compliance and safety. “High flow” can mean any gas flow with a flow rate higher than some other threshold flow rate that is relevant to the context—for example, where providing a gas flow to a patient at a flow rate to meet inspiratory demand, that flow rate might be deemed “high flow” as it is higher than a nominal flow rate that might have otherwise been provided. “High flow” is therefore context dependent, and what constitutes “high flow” depends on many factors such as the health state of the patient, type of procedure/therapy/support being provided, the nature of the patient (big, small, adult child) and the like. A person skilled in the art would appreciate, in a particular context what constitutes “high flow”. But, without limitation, some indicative values of high flow can be as follows.

In some configurations, high flow delivery of gases to a patient at a flow rate of greater than or equal to about 5 or 10 litres per minute (5 or 10 LPM or L/min).

In some configurations, high flow delivery of gases to a patient is at a flow rate of about 5 or 10 LPM to about 150 LPM, or about 10 LPM to about 120 LPM, or about 15 LPM to about 95 LPM, or about 20 LPM to about 90 LPM, or about 20 LPM to about 70 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied by embodiments of the systems disclosed, may comprise, but is not limited to, flows of at least about 5, 10, 15, 20, 30, 40, 50, 60, 70, 80, 90, 100, 110, 120, 130, 140, 150 LPM, or more, and useful ranges may be selected to be any of these values (for example, about 20 LPM to about 90 LPM, about 15 LPM to about 70 LPM, about 20 LPM to about 70 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM). Thus, ‘high flow’ or ‘high flow respiratory support may refer to the delivery of gases to a patient at a flow rate of between about 5 or 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM.

In “high flow” the gas delivered will be chosen depending on for example the intended use of a therapy or support. Gases delivered may comprise a percentage of oxygen. In some configurations, the percentage of oxygen in the gases delivered may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “High flow” for premature/infants/paediatrics (with body mass in the range of about 1 to about 30 kg) can be different. The flow rate can be set to about 0.4 LPM/kg to about 8 LPM/kg with a minimum of about 0.5 LPM and a maximum of about 70 LPM. For patients under 2 kg maximum flow may be set to 8 LPM.

High flow may be used as a means to promote gas exchange and/or respiratory support through the delivery of oxygen and/or other gases, and through the removal of CO₂ from the patient's airways. High flow may be particularly useful prior to, during or after a medical procedure. Further advantages of high gas flow can include that the high gas flow increases pressure in the airways of the patient, thereby providing patency support that opens airways, the trachea, lungs/alveolar and bronchioles. The opening of these structures enhances oxygenation, and to some extent assists in removal of CO2.

The increased pressure can also keep structures such as the larynx from blocking the view of the vocal chords during intubation. When humidified, the high gas flow can also prevent airways from drying out, mitigating mucociliary damage, and reducing risk of laryngospasms and risks associated with airway drying such as nose bleeding, aspiration (as a result of nose bleeding), and airway obstruction, swelling and bleeding.

In this specification, the terms subject and patient are used interchangeably. A subject or patient may refer to a human or an animal subject or patient.

In this specification, it is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### Overview

Embodiments of the present disclosure provide systems and apparatus that provide for integration of different forms of respiratory support into a single system, or provide integrated switching between separate systems or apparatuses that enables convenient use of those systems or apparatuses by a clinician user who may desire to switch between different forms of respiratory support delivered to a patient. Aspects of the disclosure relate to various systems, devices, apparatuses, switching mechanisms and lumen assemblies as well as systems incorporating humidification. It is to be understood that one of skill in the art would appreciate that various features and advantages described in the context of one aspect have utility in the context of another aspect and that such combinations are within the scope of and expressly form part of this disclosure.

System Switching with Gas Control

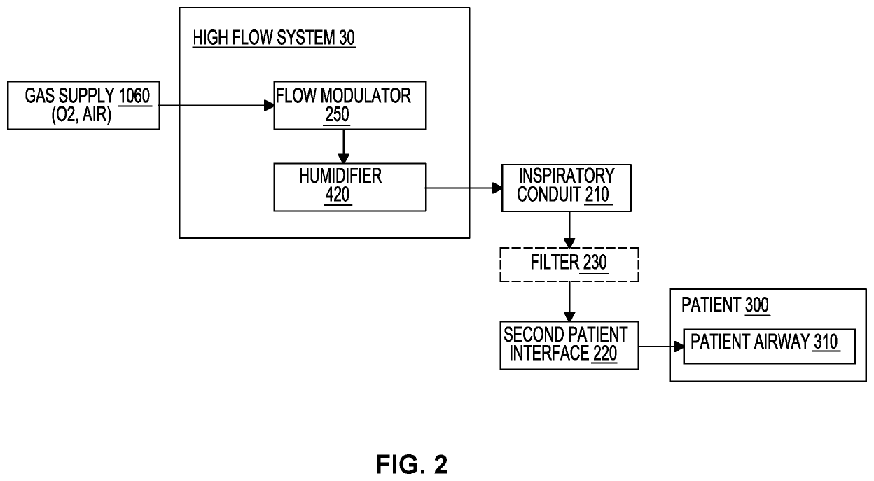

FIG. 3 is a schematic illustration showing components in a system 1000 for delivering breathing gases to a patient 300. The system 1000 includes a first respiratory apparatus 100 configurable to deliver breathing gas including one or more anaesthetic agents to the patient and a second respiratory apparatus 200 configurable to deliver breathing gas to the patient at a pre-determined flow rate. First respiratory apparatus 100 may incorporate one or more components of an anaesthesia device 10 (FIG. 1A) and second respiratory apparatus 200 may incorporate one or more components of a high flow system 30 (FIG. 2). For simplicity, like numerals are utilised to denote such components throughout this disclosure.

A switching means 700 is operable to select a mode of operation of the system 1000, said mode of operation being selected from a group including a first mode in which breathing gases are delivered to the patient by the first respiratory apparatus 100 and a second mode in which breathing gases are delivered to the patient by the second respiratory apparatus 200 at the predetermined flow rate which is typically in a range of about 20 LPM to about 90 LPM for most patients.

FIG. 3 shows switching means 700 in broken line designating flexibility in the manner in which the switching means 700 is deployed to provide for switching between the first mode and the second mode. In some embodiments, the switching means 700 is arranged between the gas supply 1060 and the first and second apparatuses 100, 200 and/or may include one or more elements forming part of the first apparatus or the second apparatus as will be exemplified by reference to various embodiments described herein. Thus, while switching means 700 is shown schematically as a box feature in FIG. 3, it is to be understood that the switching means may be brought into effect, in that it consists of or includes, one or more switching mechanisms configured to alter flow of breathing gas in system 1000 according to selection of the first mode or the second mode.

A switching mechanism may in turn consist of or include one or more switching elements. Thus the switching means 700 may include a user operable actuator which enables a user to manually select the mode of operation and in turn, causes operation of system components in the selected mode, and/or a sensor driven automated system that operates the system components in a mode which is determined by a sensor detecting e.g. which of the first patient interface 100 and the second patient interface 200, is coupled with the patient's airway 310. Various switching elements may be operatively coupled to actuate substantially simultaneously, or in response to other switching elements or actuators or under control of a controller as will become apparent by reference to the non-limiting examples provided.

While various embodiments are disclosed herein that prevent delivery of anaesthetic agents to the patient when the second mode is selected, it is to be understood that yet other approaches may be deployed, as alternatives or in addition to the examples shown in the Figures. For example, the system 1000 may inactivate release of anaesthetic agents to the patient by shutting down vaporizers or reducing their function to a level that is ineffective. Alternatively or additionally the system 1000 may inactivate delivery of anaesthetic agents to the first respiratory apparatus 100. Alternatively or additionally the system may inactivate anaesthetic agents in a flow of breathing gases delivered from the first respiratory apparatus 100 by use of a neutralizer in the first respiratory apparatus that becomes activated when the system is operated in the second mode such that any anaesthetic agents that may be flowing through the system are rendered ineffective. While wasteful this can be an important safety measure.

When the first mode is selected, the system 1000 directs breathing gases in a first inspiratory flow path 110, in which a first patient interface 120 directs breathing gases into an airway 310 of the patient 300. The first patient interface 120 is a sealing interface such as a sealing mask or endotracheal tube and is configured to direct expired gases from the patient to an expiratory flow path 130 which returns the expired gases to the first respiratory apparatus 100. Returned expired gases are treated by rebreathing components 140 as described in relation to FIG. 1.

When the second mode is selected, the system 1000 isolates flow of breathing gases from the first respiratory apparatus 100 so as to prevent delivery of anaesthetic gas including NO and vaporized anaesthetic agents to the patient 300. Thus when the second mode is selected, the system directs flow of breathing gases in a second inspiratory flow path 210 in which a second patient interface 220 directs the breathing gases into an airway 310 of the patient 300 and is a non-sealing interface. Typically, the second patient interface is a nasal cannula having one or more nasal prongs directing gases into one or both nares of the patient.

In one embodiment, the switching means 700 includes a switching mechanism located between a gases supply 1060 and the first and second respiratory apparatuses 100, 200 and includes a gas delivery apparatus which receives a supply of gases including NO, O₂ and/or air and provides a flow meter to control the flow rate of gases through one or more breathing gas outlets. Preferably, the flow meter controls flow of breathing gases through the one or more breathing gas outlets responsive to selection of the first mode of operation or the second mode of operation. This may be brought into effect in several ways.

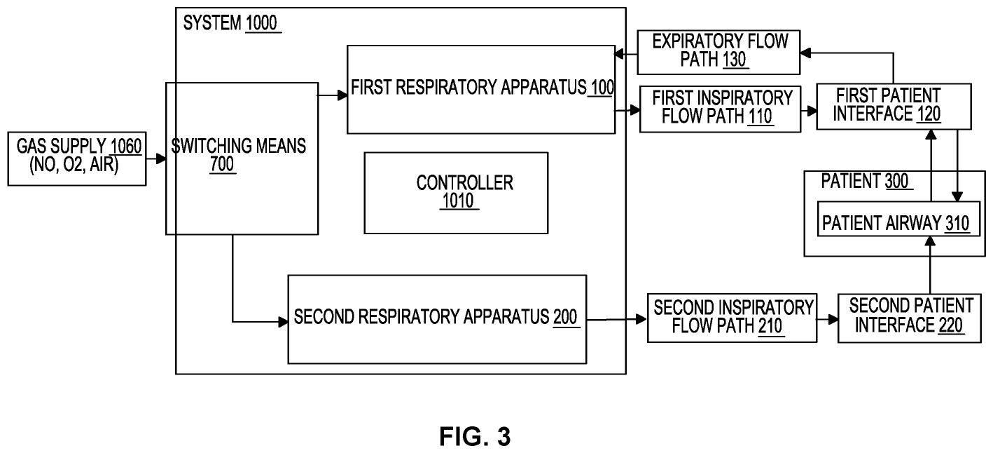

In one example shown schematically in FIGS. 4A and 4B, gas delivery apparatus 1040 includes a gas mixing element 1042 for combining NO, O₂ and air in a proportion required for operation of the system in the first mode. Flow meters may be incorporated into the gas supply 1060, or placed upstream of the gas delivery apparatus 1040, or incorporated into it, to control the proportion of gases entering gas mixing element 1042. Typically such flow meters are manually controlled e.g. by a proportional valve with rotary actuator, but may also be precision controlled by a controller 1010 of the system 1000. Safety features may be built in to limit flow rates and proportions of gases to be within safe limits, for example to ensure the ratio of O₂ to NO does not decrease below 0.25.

Flow meter 1090 controls flow rate of breathing gas from the gas mixing element 1042. First gas outlet 1044A supplies breathing gas from the gas delivery apparatus 1040 to the first respiratory apparatus 100 when the first mode is selected (FIG. 4A), and second gas outlet 1044B supplies breathing gas from the gas delivery apparatus to the second respiratory apparatus 200 when the second mode is selected. (FIG. 4B). To achieve this, a first switching element 710 may be operatively coupled with the switching means 700 and may be operable to permit flow of NO into the gas mixing element 1042 when the first mode is selected (FIG. 4A), and to preclude flow of NO into the gas mixing element when the second mode is selected (FIG. 4B). Simultaneously with operation of the switching means 700 to select the first mode, flow meter 1090 may be operatively coupled with the switching means 700 and may limit rate of flow to a low rate of up to 15 LPM, preferably 10 to 15 LPM and with operation of the switching means 700 to select the second mode, flow meter 1090 may increase rate of flow to up to 90 LPM, preferably 40 to 70 LPM. A second switching element 720 operatively coupled with the switching means 700 is operable to control gas flow from the gas delivery apparatus 1040 to one of the first and second gas outlets 1044A, 1044B. When the first mode is selected, breathing gas (including NO) from the gas delivery apparatus 1040 is directed only to the first gas outlet 1044A as represented by solid lines in FIG. 4A. When the second mode is selected, breathing gas from the gas delivery apparatus which excludes NO is directed only to the second gas outlet 1044B as represented by solid lines in FIG. 4B.

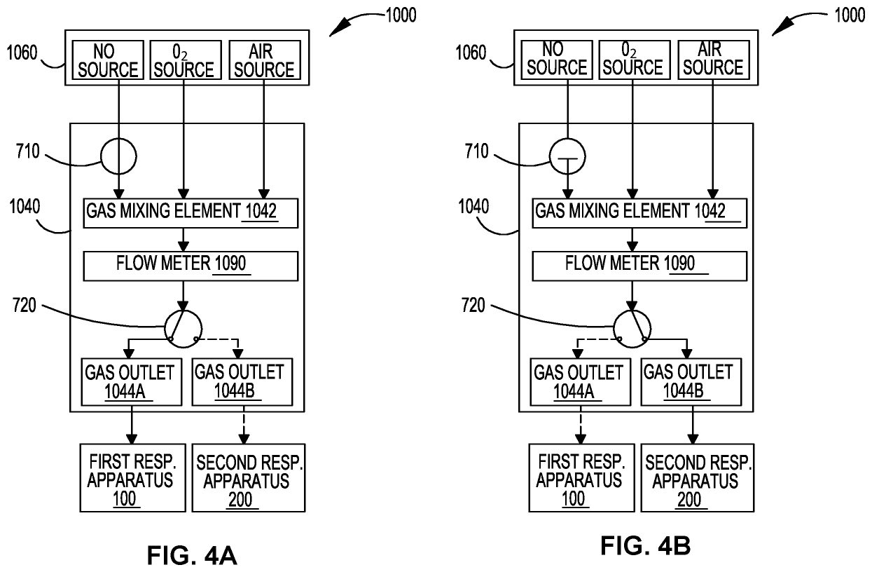

In another example shown schematically in FIGS. 5A and 5B, gas delivery apparatus 1040 includes a single, common gas outlet (CGO) 1044 supplying breathing gas from the gas delivery apparatus to the first and second respiratory apparatuses 100, 200 of the system 1000. First switching element 710 operatively coupled with the switching means 700 is operable to control input to the common gas outlet 1044 such that when the first mode is selected (FIG. 5A), the common gas outlet receives breathing gas from the gas mixing element which can combine NO, air and O₂; and when the second mode is selected (FIG. 5B), the common gas outlet receives breathing gas from the flow meter 1090. A second switching element 720 coupled with the switching means 700 is operable to control gas flow from the CGO 1044 to either the first respiratory apparatus 100 or the second respiratory apparatus. When the first mode is selected, breathing gas (including NO) from the gas delivery apparatus 1040 is directed from the gas mixing element to the CGO) 1044 and to the first respiratory apparatus 100 as represented by solid lines in FIG. 5A. When the second mode is selected, breathing gas comprising O2 (and optionally excluding NO) is directed from the flow meter 1090 to the CGO 1044 and to the second respiratory apparatus 200 as represented by solid lines in FIG. 5B. It is to be understood that in some embodiments, when the second mode is selected, breathing gas comprising air and O2 (and optionally excluding NO) is directed from the flow meter 1090 to the CGO 1044 and to the second respiratory apparatus.

In FIGS. 4A to 5B, the first and second switching elements 710, 720 may be operatively coupled so that they operate substantially simultaneously with or following operation of the switching means e.g. a knob or actuator operable by a user or an electronic controller of the system. Alternatively, the switching means 700 may incorporate the first and second switching elements 710, 720 such that they are incorporated into a common mechanical or pneumatic actuator which is operable by a user to trigger the first and second switching elements 710, 720. The first and second switching elements may include e.g. one or more gas flow or diverter valves.

In the embodiments shown in FIGS. 4A to 5B, first respiratory apparatus 100 and the second respiratory apparatus 200 may be integrated in a unitary machine. This provides a convenient complete respiratory support system 1000 which provides the capability to deliver anaesthesia to the patient in the first mode, further supported by high flow respiratory support in the second mode which can be beneficial e.g. when preparing to intubate the patient, or when weaning the patient off sedation. This arrangement conveniently places features for user control of the second respiratory apparatus 200 delivering high flow respiratory support together with features for user control of the first respiratory apparatus 100 delivering sedation. The unitary machine also simplifies and reduces the instrumentation that occupies valuable space in the clinical environment.

In some embodiments, the unitary machine may include a humidifier (not shown), typically located between the flow meter and the second patient interface 220 delivering gases from the second respiratory apparatus 200. The humidifier is configured to condition the breathing gas to a pre-determined temperature and/or humidity before delivery to the patient in the second mode. This has the advantage of streamlining set-up of the humidifier by integrating it into the daily routine of setting up the integrated system 1000.

However, it is to be understood that the switching mechanism described could similarly be deployed in a system 1000 in which the first respiratory apparatus 100 and second respiratory apparatus 200 are separate machines. Beneficially, the switching means 700 as described provides integrated control of the operation of these machines such that a single switching input simultaneously permits delivery of gas to the patient from the first respiratory apparatus 100 and prevents delivery of gas from the second respiratory apparatus 200 when the first mode is selected, or the vice versa when the second mode is selected.

Advantageously, the embodiments shown in FIGS. 4A to 5B remove the need to set up an additional oxygen supply; both the first respiratory apparatus 100 and the second respiratory apparatus 200 receive oxygen from a common supply 1060. Furthermore, this arrangement facilitates simultaneous switching to both the first respiratory apparatus 100 and the second respiratory apparatus 200 such that when the second mode is selected, the first respiratory apparatus 100 ceases to deliver gases to the patient 300. This provides improved safety by preventing delivery of anaesthetic agents including NO and volatile anaesthetics vaporized by the first respiratory apparatus in the flow of gases delivered to the second patient interface 220. Furthermore, it does not allow anaesthetic agents to enter the environment avoiding inhalation of these agents by carers attending to the patient, while also reducing wastage.

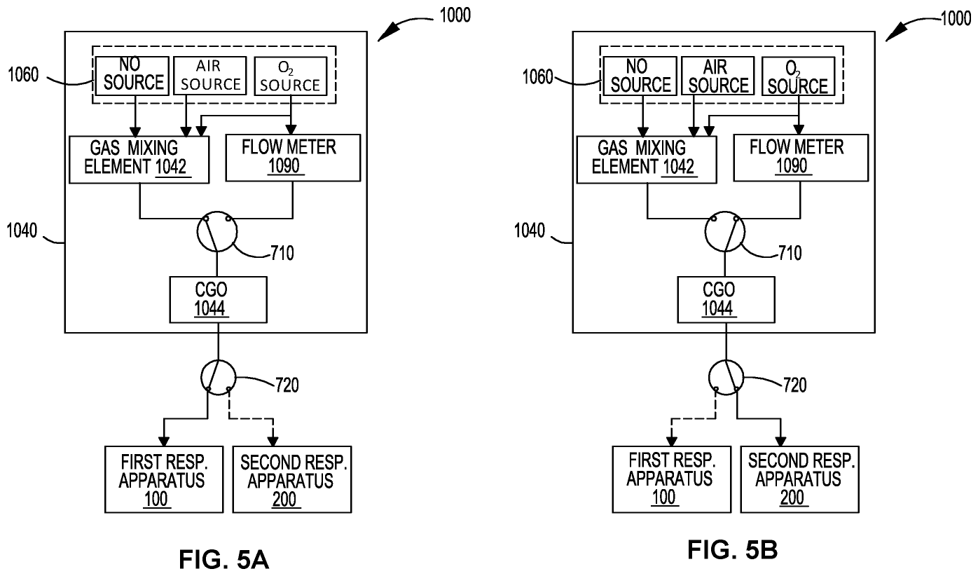

In another example shown schematically in FIGS. 6A and 6B, gas delivery apparatus 1040 provides a first switching element 710 which controls flow of breathing gas (including anaesthetic agents) from the gas delivery apparatus to the first respiratory apparatus 100, and a second switching element 720 outside the gas delivery apparatus which controls flow of breathing gases through the second respiratory apparatus 200. As depicted by the dot-dash line, switching elements 710 and 720 are operatively linked so as to operate substantially simultaneously upon selection of the required mode of operation by switching means 700. In the first mode depicted in FIG. 6A, first switching element 710 is open and second switching element 720 is closed. This permits flow from gas supply 1060 to gas mixing element 1042 and into gas outlet 1044 which supplies first respiratory apparatus 100, while preventing flow through second respiratory apparatus 200. In the first mode of operation, breathing gases including anaesthetic agents are delivered to the patient by first patient interface 120 which also receives expired gases from the patient and returns them to rebreathing components 140 of the first respiratory apparatus via expiratory conduit 130.

In the second mode of operation, depicted in FIG. 6B, the first switching element 710 is closed and second switching element 720 is open. This prevents flow of gases to gas outlet 1044 which in turn prevents delivery of breathing gases including anaesthetic agents from the first respiratory apparatus 100 to the patient. Meanwhile, flow meter 1090 receives oxygen (and optionally, air) from gas supply 1060 and increases the rate of flow to a predetermined rate of up to 90 LPM, preferably 40 to 70 LPM. The high flow of gas from flow meter 1090 is preferably passed through humidifier 420 and delivered to the patient 300 via second patient interface 220. In this arrangement the flow meter 1090 forms a key component of the second respiratory apparatus 200, together with preferred humidifier 420, as depicted by the broken line encircling these features. An advantage of providing second switching element 720 downstream from flow meter 1090 is that the flow meter does not require time to wind up to the predetermined high flow rate when the second mode is selected. It is to be understood, however, that second switching element 720 can be located upstream of the flow meter 1090 or downstream of the humidifier 420.

Switching System with Manifold

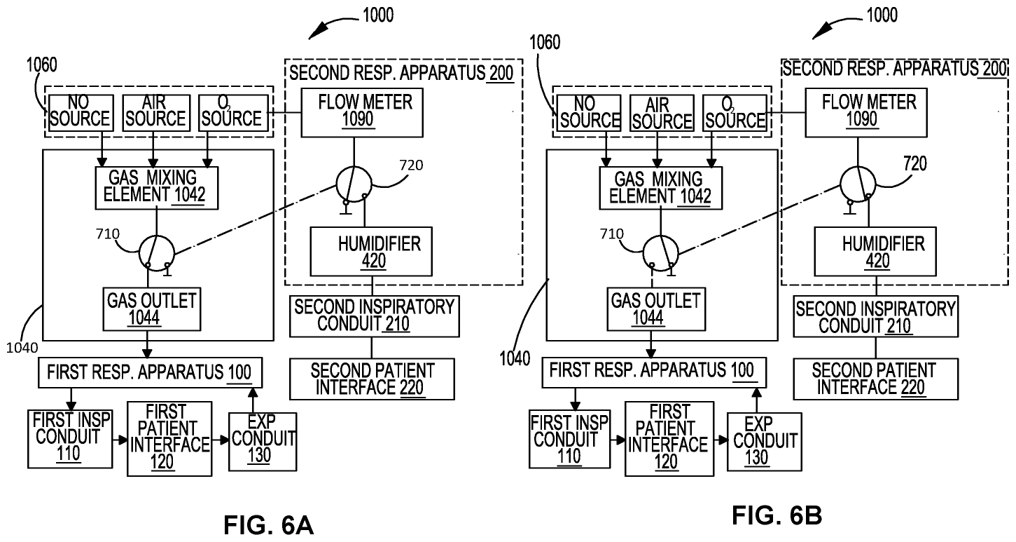

In another example of a system 1000 in which the switching mechanism 700 is located between gas source 1060 and the first and second respiratory apparatuses 100, 200, a gas diverter receiving a supply of gases including anaesthetic gas and a breathing gas contains two switching means operable to control flow of gases to the first and second respiratory apparatuses. An example of a gas diverter 800 is depicted schematically in FIGS. 7A and 7B. The gas diverter 800 receives a supply of gases including NO, O₂ and/or air. Gas diverter 800 has a first switching element 710 which controls flow of NO from the gas supply 1060 to an outlet 820 and a second switching element 720 which controls flow of breathing gas, namely O₂ to an outlet 822 or 828. In some examples, the breathing gas may include air, and the second switching element consists of paired elements 720A and 720B which cooperate to direct the flow of O₂ and air to outlets 822/828 and 824/826 respectively. As depicted by the broken lines inside gas diverter 800, switching elements 710 and 720A,B are operatively linked so as to operate substantially simultaneously upon selection of the required mode of operation by switching means 700. First and second switching elements 710, 720 are operatively coupled either by physical (e.g. pneumatic, magnetic, mechanical) or electronic means such that operation of one switching element occurs substantially simultaneously with the other switching element.

In the first mode depicted in FIG. 7A, the first switching element 710 (which may be e.g. a flow control valve) is open allowing flow of NO to outlet 820. Meanwhile, second switching element 720A,B (which may be e.g. a gas diverter) directs breathing gases (O₂ and air) to outlets 822 and 824 respectively. In the second mode depicted in FIG. 7B, the first switching element 710 is closed, preventing flow of NO to outlet 820. Meanwhile, second switching element 720A,B directs breathing gases (O₂ and air) to outlets 826 and 828 respectively. Thus, operation of the switching means 700 to select the first mode provides a supply of breathing gases and NO to first respiratory apparatus 100 (typically via a flow meter), while operation in the second mode prevents supply of gas to the first respiratory apparatus. Advantageously, first switching element 710 prevents supply of NO outside the gas diverter 800 such that breathing gases delivered to the second respiratory apparatus 200 exclude anaesthetic agents when the gas diverter is in the second configuration.

Advantageously, the gas diverter manifold 800 is installed between a gas supply 1060, such as a gas wall supply in an operating theatre (or other medical location) and the gas inlet ports of the first respiratory apparatus 100 which are in fluid communication with vaporizer 150 (see FIG. 1). The gas diverter manifold 800 is also installed between the gas supply 1060 and the gas inlet ports of the second respiratory apparatus. Activation of the second mode automatically causes cessation of supply of NO to the first respiratory apparatus 100 and diversion of oxygen and air to the second respiratory apparatus 200. Advantageously, the gas diverter 800 could be supplied as a standalone component that could be retrofitted to existing anaesthesia machines or it could be incorporated into newly built machines. In some embodiments, during cessation of supply of NO to the first respiratory apparatus 100, a small residual amount of flow of O₂ (and optionally air) may be permitted to flow from the gas diverter manifold 800 to the first respiratory apparatus. This may be desirable in scenarios where existing anaesthesia machines to which the gas diverter 800 has been retrofitted are configured to alarm if the machine detects that it is not receiving a flow and/or pressure from an O2 or air supply.

### Gas Diverter as Standalone

While the gas diverter 800 has been described in the context of a component of the system 1000, it is to be understood that gas diverter 800 may be supplied as a standalone device for use with a respiratory system such as a system providing the functionality of the first respiratory apparatus 100 (typically an anaesthesia machine) and the second respiratory apparatus 200 (delivering high flow respiratory support). Although the gas diverter 800 depicted in FIGS. 7A and 7B provides three inlets, three outlets to the first respiratory apparatus 100 and two outlets to the second respiratory apparatus 200, it is to be understood that the air inlet and outlets may be omitted such that gas diverter 800 receives a flow of NO and O2 only. Gas diverter 800 includes a manifold between the inlets and the outlets which provides the required flow paths for delivery of gases to the first and second respiratory apparatuses 100, 200 in the first and second modes respectively. In some embodiments, the manifold may combine the flow of NO and O₂ so that they are delivered to the first respiratory apparatus 100 via a single common gas outlet. Thus, in one embodiment, gas diverter 800 includes a first outlet (combining 820/822), a second outlet 828 and a manifold between the first and second inlets and the first and second outlets which provides a first flow path to the first outlet and a second flow path to the second outlet. In this embodiment, gas delivery device 800 is operable in a first configuration wherein the first flow path is open and the second flow path is closed and a second configuration in which the second flow path is open and the first flow path is closed. In the first configuration (FIG. 7A), the gas delivery device 800 prevents flow of anaesthetic gas to the outlets.

As disclosed in the context of FIGS. 7A and 7B, gas delivery device 800 includes one or more switching elements in the manifold for creating the first and second flow paths. The one or more switching elements may include first valve 710 controlling flow of anaesthetic gas (NO) in the manifold such that in the first configuration the first valve 710 is open and directs NO to the first flow path, and in the second configuration (FIG. 7B) the first valve 710 is closed. The switching elements may include second valve 720 controlling flow of breathing gas (O₂) in the manifold such that in the first configuration the second valve 720 directs breathing gas to the first flow path, and in the second configuration the second valve 720 directs breathing gas to the second flow path. Ideally one or both of the first and second valves are operable to control rate of flow of gas therethrough. When a third gas, such as room air or high pressure air from a flow modulator 250, is delivered through gas delivery device 800, the manifold may combine the breathing gases (air and O₂) so that second valve 720 (depicted as 720A,B) directs breathing gases to a single outlet 828 delivering breathing gas to the second respiratory apparatus in the second configuration. Ideally, the second valve is operable to control O2 concentration in breathing gas delivered to the first and second flow paths. This may be done directly by controlling the proportion of O2 gas flowing through the manifold, or indirectly by controlling rate of flow of O2.

In some embodiments, gas delivery device 800 is operable in a third configuration in which both the first flow path and the second flow path are open and a stop-cock or other blocking mechanism may be utilised at the patient end to preclude flow though the first or second patient interface. A gas mixer (not shown) may also be included for combining received gases in a proportion required to deliver a required therapy. The gas delivery device 800 may include a switching means operable by a user to select a configuration for operation of the gas delivery device which is achieved by switching elements (e.g. valves and gas flow diverters) in the manifold. The switching means may be located e.g. on the gas diverter device 800, the first respiratory apparatus 100 or on the second respiratory apparatus 200, or on a patient interface through which breathing gas is directed into an airway of the patient.

The switching means may be pneumatic, mechanical, electronic or utilise any other mechanism that is suitable to trigger operation of the switching elements. In an embodiment utilising electronic switching means, gas delivery device 800 is configured for connection to a power source and the power source may include a battery. Ideally both mains power and battery supplies are utilised, such that in the event of power outage, the battery is charged and ensures continued operation of the gas delivery device until conclusion of the procedure or until mains power is restored.

In some embodiments, the switching means is activated in response to detection of a change in state detected by one or more system sensors. These sensors may include one or more of e.g. a pressure sensor, CO₂ sensor, O₂ sensor, flow sensor, gas concentration sensor or the like and they are ideally located and configured to determine if a breathing circuit delivering gases to the patient airway is associated with a first patient interface by which gases (e.g. including anaesthetic agents) are delivered to the patient airway by a substantially sealed interface, or associated with a second patient interface by which gases excluding anaesthetic agents are delivered to the patient in by a non-sealing interface. The switching means may be in wired or wireless communication with one or more switching elements to control operation of the gas delivery device according to the configuration selected by the user.

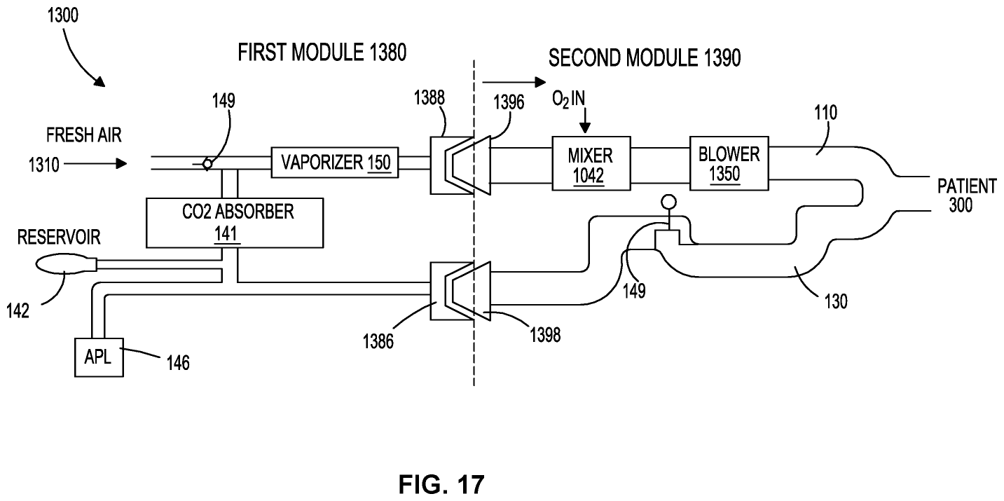

In some embodiments, gas delivery device includes an output module (akin to monitor 1094 in FIG. 20) providing one or both of a visible and audible indication of the configuration in which the gas delivery device is operating. The output module may also present to the user other parameters relevant to use of the gas delivery device or the respiratory system as a whole, such as flow rates and gas concentrations. Operation of the output module may be activated e.g. when switching means is operated by the user to select the second configuration, or it may have a button or actuator to switch on and off with visible and/or audible indications being provided at all relevant times while switched on.

O₂ switching

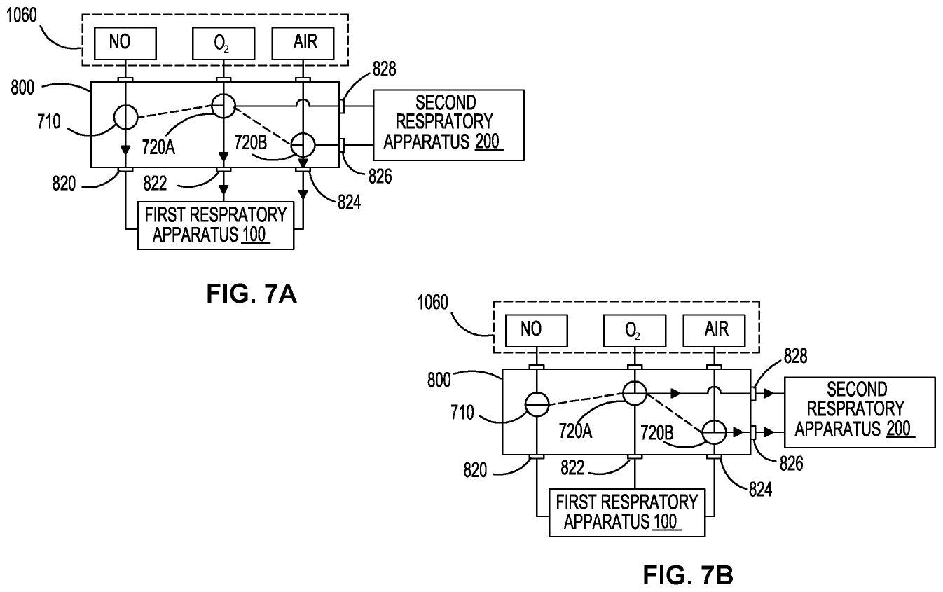

In another example of a system 1000 in which the switching mechanism 700 is located between a gas source 1060 and the first and second respiratory apparatuses 100, 200, the switching mechanism includes a first switching element 710 operable to direct flow of O₂ to the first respiratory apparatus 100 in the first mode and to the second respiratory apparatus 200 in the second mode. FIG. 8 provides a schematic illustration depicting operation of such a switching mechanism 700 in the first mode. Notably, in the first mode a second switching element 720 is also open and operable, in a preferred embodiment, to permit the first respiratory apparatus 100 to perform in either a manual (147) or automatic (148) ventilation/rebreathing mode. In the embodiment shown in FIG. 8, the manual first mode has been selected. Second switching element 720 is responsive to the first switching element 710. Therefore, when the first switching element 710 is switched to the second mode, O₂ is directed to the second respiratory apparatus 200 (represented by flow modulator 250 and humidifier 420) and the second switching element moves to the lowest position 722 closing off flow from the first apparatus rebreathing components 140 to the patient 300. In some embodiments, when the first switching element 710 is switched to the second mode, O₂ and/or air may be directed to the second respiratory apparatus 200. Since operation of the first switching element 710 in the second mode prevents flow of O₂ into the gas mixing element 1042 of first respiratory apparatus 100, there is also no flow to the patient 300 through first patient interface.

As in the embodiments exemplified in FIGS. 4A to 5B, the arrangement of FIG. 8 removes the need to set up an additional oxygen supply; both the first respiratory apparatus 100 and the second respiratory apparatus 200 receive oxygen from a common supply 1060. Furthermore, this arrangement facilitates simultaneous switching to both the first respiratory apparatus 100 and the second respiratory apparatus 200 such that when the second mode is selected, the first respiratory apparatus 100 ceases to deliver gases to the patient 300. This provides improved safety by preventing delivery of anaesthetic agents including NO and volatile agents vaporized by vaporizers 150 of the first respiratory apparatus into the flow of gases delivered to the second patient interface 220. Furthermore, it does not allow anaesthetic agents to enter the environment avoiding inhalation of these agents by carers attending to the patient, while also reducing waste.

The embodiments shown in FIGS. 6A to 8 have particular utility in systems 1000 in which first respiratory apparatus 100 and the second respiratory apparatus 200 are separate machines. Ideally, the second respiratory apparatus 200 in each case has a humidifier 420 (specifically shown in FIG. 8) to condition the breathing gas to a pre-determined temperature and/or humidity before delivery to the patient in the second mode. Furthermore and as previously stated, the first and second switching elements 710, 720 are operatively coupled for substantially simultaneous operation with or following operation of the switching means 700 although it is to be understood that in some embodiments, the switching means may incorporate the first and second switching elements such that they are incorporated with a common mechanical or pneumatic actuator which is operable by a user to trigger the first and second switching elements. The first and second switching elements 710, 720 may include one or more gas flow or diverter valves.

### Switching Interface/Control

In some embodiments, including in the context of the examples already described, it may be desirable that the predetermined flow rate of breathing gas delivered to the patient 300 in the second mode is selectable from a range of from about 20 LPM to about 90 LPM by the user operating the switching means although in some cases such as paediatric or neonatal patients, a lower range may be desired. Preferably, the required pre-determined flow rate is selectable by the user from a plurality of pre-determined available flow rates such as e.g. 0 LPM, 40 LPM and 70 LPM although it is to be understood that additional and/or different predetermined rates may be selectable within the high flow ranges disclosed herein.

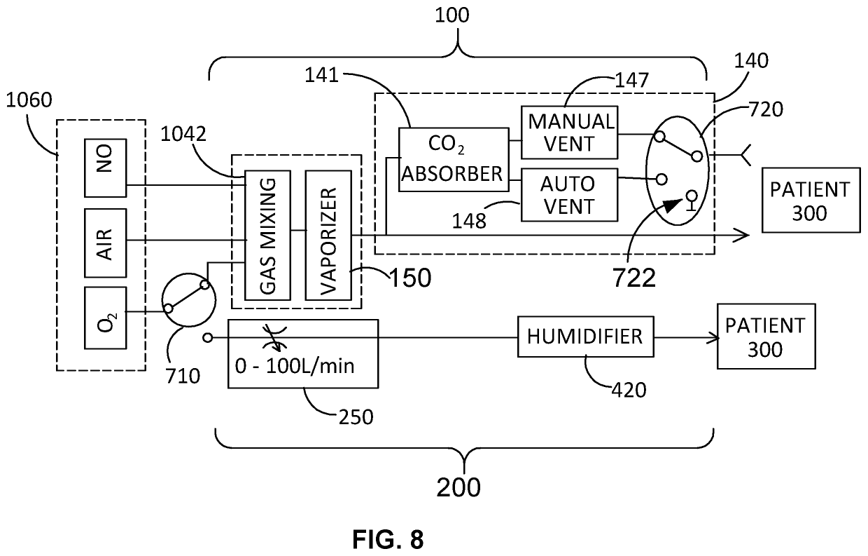

Selection of the required predetermined rate may be achieved by operation of the switching means 700 which in some embodiments includes a rate selector in the form of e.g. a knob, sliding switch, touch screen or other actuator which provides for user selection of the required predetermined flow rate from a plurality of predetermined flow rates. An example of a rate switching element 730 associated with a rate selector is presented in the schematic illustration of FIG. 9 which is similar to FIG. 8, with the exception that flow modulator 250 has been replaced with two flow modulators 250A, 250B operating at e.g. 70 LPM and 40 LPM respectively, which are in fluid communication with rate switching element 730 which also has an “off” position 732. In some embodiments, rate switching element 730 may replace first switching element 710 however in some embodiments it may be desirable to provide rate switching element 730 in addition to first switching element 710 to avoid issues of delay in gas flow rate wind up when the second mode is selected. Preferably rate switching element 730 is operatively coupled with the second switching element 720 so that operation of the rate selector to deliver flow from the second respiratory apparatus 200 (i.e. through humidifier 420 to the second patient interface as shown) in the second mode prevents delivery to the patient of breathing gases from the first respiratory apparatus.

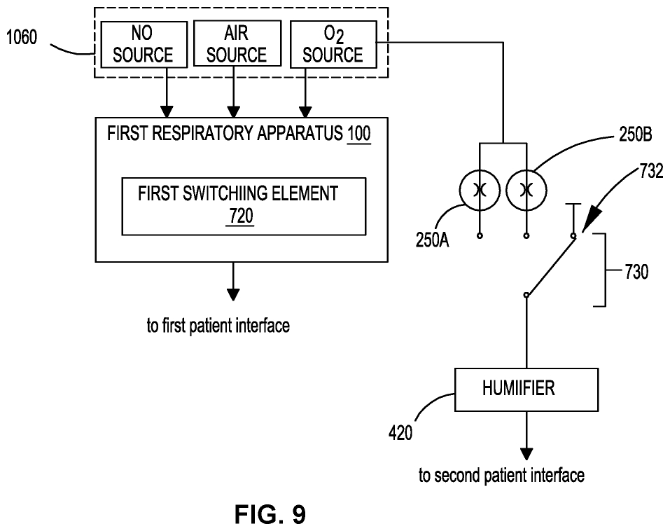

In some embodiments operation of the rate switching element 730 to select a rate of 0 LPM permits supply of O₂ to the first respiratory apparatus. This is shown schematically in FIG. 10 in which rate switching element 730 controls flow of O₂ to the first and second respiratory apparatuses 100, 200. Thus, when the rate switching element 730 is operated to select one of the two predetermined flow rates (exemplified as 40 LPM and 70 LPM) there is flow of O₂ to the second respiratory apparatus i.e. in the second mode, and when the rate switching element 730 is operated to select 0 LPM, flow from the O₂ supply is directed to the first respiratory apparatus 100, i.e. in the first mode. Rate switching element 730 and second switching element 720 are operatively coupled such that operation of the rate selector in the second mode activates switching of the second switching element to the lowest position 722 closing of flow from the first apparatus rebreathing components 140 to the patient 300. Since operation of the rate switching element 730 in the second mode prevents flow of O₂ into the gas mixing element 1042 of first respiratory apparatus 100, there is no flow to the first patient interface in the second mode.

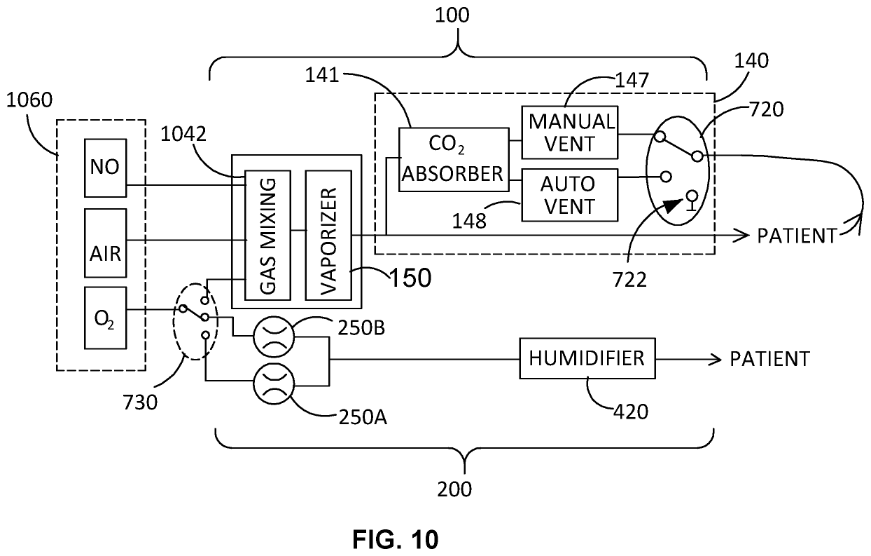

In another example involving a rate selector, a pressure controlled actuator is deployed to control of flow to the first and second respiratory apparatuses 100, 200 as shown in the schematic illustration of FIG. 11. In this arrangement, pressure controlled actuator 740 detects back flow pressure from second respiratory apparatus 200. During operation in the second mode, i.e. when rate switching element 730 is operated to select an available non-zero flow rate, the pressure controlled actuator prevents flow of gases to the first respiratory apparatus 100. When rate switching element 730 is operated to select 0 LPM flow rate, back pressure from the second respiratory apparatus 200 increases triggering pressure controlled actuator 740 to direct flow of gases to first respiratory apparatus 100.

### 3 Position Bag/Vent/HF Switch

In some embodiments, switching means 700 provides yet further functionality for system 1000 in that it can provide for selection by the user of a manual first mode of operation or a mechanical first mode of operation of the first respiratory apparatus, as well as operation of the system in the second mode. In the manual first mode of operation the first respiratory apparatus 100 deploys a ventilation bag for manual ventilation of the patient 300 e.g. during intubation, with a medical professional manually squeezing the bag to control the timing and tidal volume of the breathing gases delivered to the patient's airway by the first patient interface. In the mechanical first mode of operation the first respiratory apparatus 100 deploys the bellows to provide for mechanical ventilation of the patient 300, e.g. once sedated, wherein the bellows control the tidal volume and timing of inhalations/exhalations. In both cases, a pressure relief valve is provided to avoid over pressure in the system in the breathing circuit connected to the patient's airway.

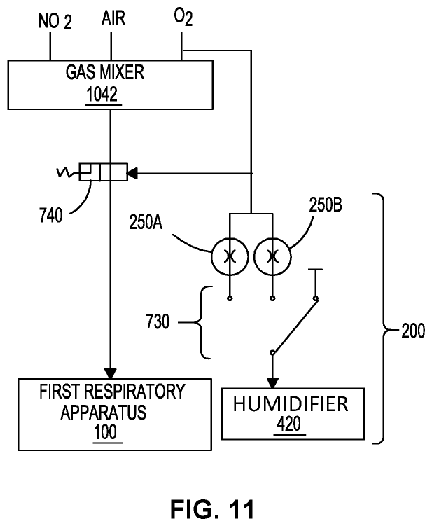

In one embodiment, the additional functionality which provides for selection of the manual first mode of operation, the mechanical first mode of operation or the second mode of operation of system 1000 is achieved by inclusion of a 3-way actuator. On example is provided in the schematic illustration of FIG. 12A which shows 3-way actuator 750 which provides fluid coupling of the rebreathing components 140 of first respiratory apparatus 100 with the ventilation bag 142 in a manual first mode or bellows 145 in a mechanical first mode, and with the second respiratory apparatus 200 in the second mode. Ideally, the first and second respiratory apparatuses receive O₂ from a common supply as described above. In order to prevent supplemental fresh gas flow (FGF) of anaesthetic agents from the vaporizer 150 contaminating breathing gases supplied to the second respiratory apparatus, a pressure controlled actuator 740 can be placed to detect a decrease in back pressure when the system 1000 is operated in the second mode, which triggers the actuator to shut off the FGF. This is shown schematically in FIG. 12B and ensures delivery of O₂ to the patient's airway via the second respiratory apparatus 200 and the second patient interface 220 in the absence of anaesthetic agents. Advantageously, use of a pressure controlled actuator, as in FIGS. 11 and 12B, provides an elegant solution the problem of contamination by FGF of gases delivered in the second mode, and avoids the need to physically or functionally couple operation of separate switching elements located in different components of the system 1000.

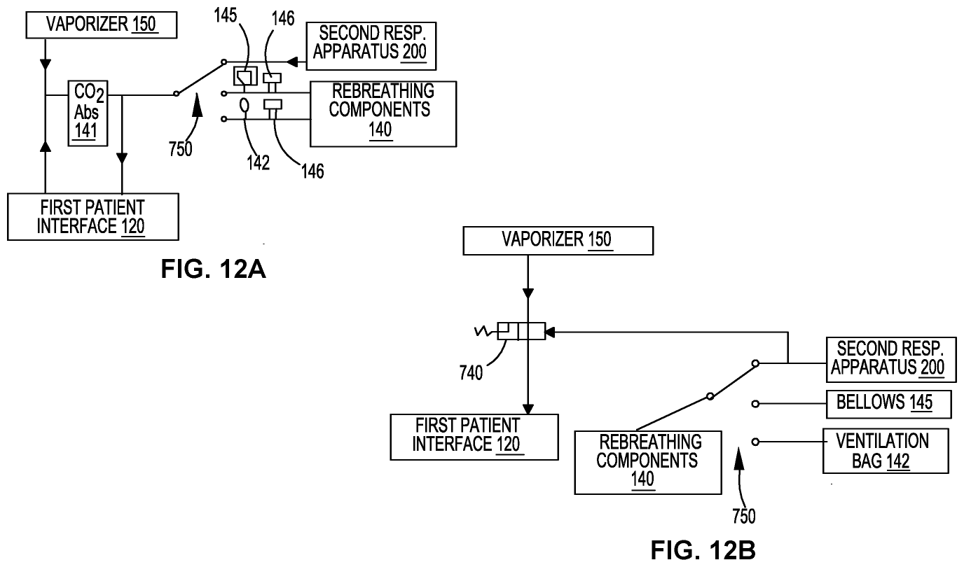

FIGS. 13A and 13B are schematic illustrations showing how a 3 position switch (FIG. 13A) could provide the physical fluid couplings required to provide for switching between the manual first mode of operation (POS I), the mechanical first mode of operation (POS II) and the second mode of operation (POS III). FIG. 13B schematically shows the breathing circuits including patient interfaces connected in each mode.

It is to be understood that while the embodiment shown in FIGS. 13A and 13B provide physical 3 position switching, an electronic 3 position switch may be utilised to yield similar control. An electronic 3 position switch may communicate with controller 1010 to control operation of the first respiratory apparatus 100 in the manual first mode or the manual second mode, or to switch to the second mode of operation in which breathing gases are delivered by the second respiratory apparatus 200. An electronic switch of this type may enjoy wired or wireless communication, the latter providing flexibility in the location of the electronic actuator which may include one or more of a button panel or touch screen attached to a housing which is removably locatable and ideally, mountable at multiple locations such as the patient's bed, the anaesthetist's person, or the hardware that is delivering gases to the patient.

An electronically enabled switch may be physically attachable to one or both of the first or second patient interfaces 120, 220 to enable quick switching of the system's mode of operation when the patient interface is swapped, e.g. when moving from pre-intubation to intubation, or when weaning the patient from sedation. Thus, a button or electronic switch located on the patient interface can be activated, causing system 1000 to select the mode of operation that will safely deliver gases to the patient through that interface. Alternatively a foot pedal or foot-operated switch or voice control may be utilised. Each of these has the potential to improve accessibility of mode selection while the anaesthetist is away from the controls located on the anaesthetic machine.

Machine Detected Changes Cause Switching on/Off NHF

While some embodiments of the system provide mechanical, pneumatic, or other physical control and operational coupling of switching elements, in some embodiments system 1000 includes a controller 1010 receiving inputs from one or more sensors deployed throughout the system. The sensors detect if a breathing circuit coupled with the patient's airway is associated with the first patient interface 120 for use with first respiratory apparatus 100 or the second patient interface 220 for use with the second respiratory apparatus 200. This information is used by the controller 1010 to operate the switching means 700 to select the mode of operation. Thus, when the sensor/s detect that the breathing circuit associated with the first patient interface 120 then the controller 1010 ensures the system is operating in the first mode of operation. Conversely, when the sensor/s detect that the breathing circuit associated with the second patient interface 220 then the controller 1010 ensures the system is operating in the second mode of operation delivering Nasal High Flow (NHF) respiratory support.

The sensors may include a pressure sensor arranged to measure back pressure in one or both of the first respiratory apparatus 100 and second respiratory apparatus 200. The system 1000 may provide a continual or intermittent flow of breathing gases through the first respiratory apparatus 100 and/or second respiratory apparatus to enable a measurement of the back pressure in the first respiratory apparatus 100 and/or second respiratory apparatus. In some embodiments, this continual or intermittent flow of breathing gases comprises a flow rate, pressure and/or volume less than the flow of breathing gases provided to the patient in the first and/or second mode. Different resistance to flow values are associated with each of the first (sealing) patient interface 120 and second (non-sealing) patient interface when coupled to the patient 300 which are used in the first and second modes respectively. When the measured back pressure indicates breathing gas is delivered to the patient by a sealing patient interface that is substantially sealed with the patient, the controller 1010 determines the breathing circuit to be associated with the first patient interface and operates the system 1000 in the first mode in which breathing gases and anaesthetic agents are deliverable to the patient 300, and expired gases returned to the rebreathing components. Alternatively, when the measured back pressure indicates breathing gas is delivered to the patient by a non-sealing patient interface, the controller 1010 determines the breathing circuit to be associated with the second respiratory apparatus and operates the system 1000 in the second mode in which breathing gases excluding anaesthetic agents are delivered to the patient at the predetermined (high flow) flow rate. In some embodiments, the pressure sensor may be located at or downstream of a flow modulator 250 of the system although the pressure sensor may be located at any convenient location in the gas flow pathway between the patient airway and the flow modulator 250.

Alternatively/additionally, the one or more sensors may include a CO₂ sensor associated with a first breathing circuit associated with the first patient interface 120 and/or a second breathing circuit associated with the second patient interface 220. In such embodiments, the controller 1010 determines the breathing circuit in which expired gas from the patient contains a higher concentration of CO₂ than ambient air to be the breathing circuit that is coupled with the patient's airway. If CO₂ concentration in both breathing circuits is higher than ambient air, the controller determines the breathing circuit with the higher CO₂ concentration to be the breathing circuit that is coupled with the patient's airway.

Alternatively/additionally, the sensors may include one or more proximity sensors such as e.g. acoustic (including audible and/or ultrasonic), optical (including infra-red), radiofrequency, pressure (in the inspiratory/expiratory conduits and/or mask cuff), flow, electrical conductivity, resistance, temperature or other sensors to determine which of the breathing circuit is coupled with the patient's airway by the first or the second patient interface. Such proximity sensors are explained in further detail in WO2016/157105A1, the contents of which are hereby incorporated herein by reference.

The switching means 700 may include one more actuators that are operable by a user, such as, but not limited to one or more of a button, switch, knob, foot operated switch or pedal. Alternatively/additionally switching means 700 may include one or more of an electronic input device, touch screen, voice activated sensor or the like, as may be operable in concert with an electronic controller of the system as will be described. One or more actuators of the switching means may be located at or near a first or second patient interface 120, 220 through which breathing gas is delivered to the patient by the first respiratory apparatus 100 or the second respiratory apparatus 100. Locating one or more of the actuators at or near the patient end of a breathing circuit delivering gases to the patient's airway provides convenience for a clinician working on the patient throughout phases of anaesthesia where it is often necessary to switch between modes of operation of the system and the form of breathing support that is delivered. Locating an actuator enabling mode selection at the patient end may be more convenient and time saving for the clinician and others in their vicinity. In other arrangements, the system 1000 is configurable to detect which of the first or second patient interfaces is attached to the patient, and modify the mode of operation accordingly.

It is to be understood that the switching means 700 may include one or more switching mechanisms such as mechanical, electronic, electromechanical, electromagnetic, pneumatic or any other suitable switching mechanisms to achieve the functionality disclosed herein. Furthermore, the one or more switching mechanisms are couplable with the switching means via wired and/or wireless coupling using techniques readily understood and ascertainable by one of skill in the art. Switching mechanisms may include actuators operable by a user of the system 1000 to select the required mode of operation and may include or consist of any of the switching mechanisms described. In some embodiments, an actuator includes an electronic input device in wireless communication with a controller 1010 of the system 1000 and movably locatable to different positions with respect to the patient 300 and/or the first and second breathing apparatuses 100, 200.

In some embodiments, the one or more switching mechanisms are operable to control one or more characteristics of breathing gases delivered to the subject, such as but not limited to presence of volatiles, flow rate, gas composition, gas concentration, temperature, and/or humidity.

For conciseness, certain features of the first respiratory apparatus 100 are not always shown in the figures. However it is to be understood that in preferred embodiments the first respiratory apparatus 100 performs the function of an anaesthetic machine 10 and includes one or more of a CO₂ absorber 141 configured to treat returned expired gas before recirculating to the patient in the first mode, a pressure limiting valve 146 configured to maintain substantially stable pressure in the system in the first mode, a variable volume 145 for displacement of gases in the first mode (e.g. using bellows or a bag ventilator), a fresh gas flow for replenishing anaesthetic gas delivered to the patient in the first mode and a vaporizer 150 for vaporizing volatile anaesthetic agents into breathing gas delivered to the patient in the first mode.

Similarly, for conciseness certain features of the second respiratory apparatus 200 are not always shown in the figures. However it is to be understood that in preferred embodiments the second respiratory apparatus 200 delivers high flow respiratory support as described herein, and includes one or more of a flow source 250 or modulator configured to generate gas flows through the system. A humidifier 420 configured to condition the breathing gas to a pre-determined temperature and/or humidity before delivery to the patient in the second mode is also typically provided although in some cases it may be omitted.

Anaesthesia machine with 3 Modes Including O₂ Flush

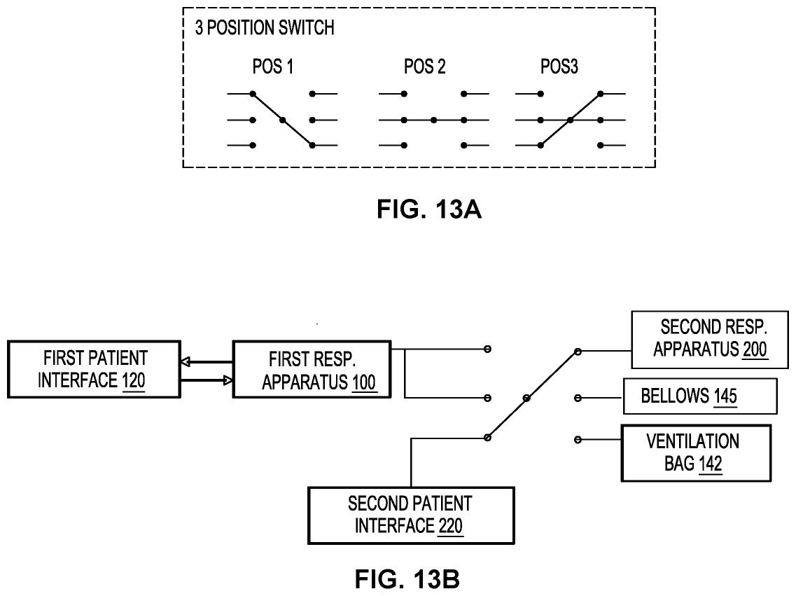

FIG. 14 is a schematic illustration of a respiratory apparatus 1000 operable to deliver breathing gases to a patient by an inspiratory gas flow path and, in some modes, to receive expired gases via an expiratory gas flow path. The respiratory apparatus is operable in a plurality of modes. In a first mode, the respiratory apparatus delivers breathing gases including one or more anaesthetic agents to the inspiratory gas flow path and receives return of expired gases via the expiratory gas flow path, typically utilizing components of first respiratory apparatus 100 as described herein. In a second mode, the respiratory apparatus disables flow of one or more anesthetic agents and delivers breathing gases including O₂ to the inspiratory gas flow path, typically utilizing components of second respiratory apparatus 200 as described herein, at a pre-determined flow rate without return of expired gas. In a transient mode the respiratory apparatus disables flow of one or more anesthetic agents and delivers high concentration O₂ to the inspiratory gas flow path.

The respiratory apparatus includes switching means operable to select one of the modes of operation from the plurality of modes. The switching means may include one or more actuators such as a button, switch, knob, foot operated switch or pedal, denoted in FIG. 14 as switching elements 710, 720 which are operatively coupled to deliver breathing gases in a first mode or a second mode in a manner akin to that described in the context of FIGS. 4A and 4B. In the first mode, the respiratory apparatus is operable to deliver breathing gases including anesthetic agents to the patient by a first patient interface forming a sealed interface with the patient's airway and returning expired gases to respiratory apparatus by the expiratory gas flow path as described herein. Preferably, the respiratory apparatus includes first respiratory apparatus 100 having features of FIG. 1A corresponding to an anesthesia machine and the first patient interface is a sealing mask or endotracheal tube.

In the second mode, the respiratory apparatus is operable to deliver breathing gases to the patient by a second patient interface, such as a nasal cannula, which forms a non-sealing interface with the patient's airway. Notably, in the first mode, FGF containing anesthetic agents from the NO source and/or vaporizer 150 enter the inspiratory gas flow path to supplement the patient's sedation. However, in the second and transient modes, flow of FGF to the second patient interface is disabled by switching element 720 preventing release of an aesthetic agents into the environment via the non-sealing interface, avoiding inhalation of these agents by carers attending to the patient, while also reducing wastage. In some configurations, the inspiratory flow path in the second and transient modes are the same. In some configurations, the inspiratory flow path in the first and third modes are the same.

In a preferred embodiment, the transient mode is activated only during operation of an actuator that is normally biased off. For example, the switching means may include a button or trigger configured to be activated by a user to operate the respiratory apparatus in the transient mode while the trigger or button is depressed and wherein release of the button or trigger disables the transient mode. Operation of the button or trigger may open flush valve 730 which permits high flows of O₂ to flow through the system, bypassing the gas mixing element 1042 and flow meter 1090. The transient mode may be used to flush the respiratory apparatus with O₂ e.g. during setup or after use. Ideally, the user can select the transient mode to flush the respiratory apparatus including the first and or second respiratory apparatuses. This may include flushing the first respiratory apparatus 100 in a manual mode of operation in which bag ventilation components are flushed, or in a mechanical mode of operation in which mechanical bellows system components are flushed. This may require manual connection of the ventilation bag for some machines although typically this is always connected with manual (bag) or mechanical (bellows) ventilation mode being selectable by a user operating a switch that alters the gas flow path to the selected ventilation components.

For operation of the respiratory apparatus in the second mode, it is desirable to configure the apparatus in gas flow communication with a flow modulator configured to provide gas flows through the system at a predetermined flow rate consistent with high flow respiratory support, and a humidifier configured to condition the breathing gas to a pre-determined temperature and/or humidity before delivery to the patient in the second mode. Such features are represented in FIG. 14 by second respiratory apparatus 200 having features of FIG. 2 together with optional filter 230. It is to be understood, however, that humidification could be provided, to a limited extent, by reconfiguring a CO₂ absorber in the first respiratory apparatus, if this is also configurable for high flow support. In this context, it is to be noted that while FIG. 14 shows two gas outlets 1044A, 1044B, a common gas outlet may supply a unitary machine providing respiratory support in the first and second modes and also configurable to provide a transient mode in which the system is flushed with O₂. This arrangement provides for use of a common gas source to operate the respiratory apparatus in the three modes removing the need to set up an additional oxygen supply to deliver high flow respiratory support and to enable O₂ flushing.

Blower Driven Anaesthesia Machine with High Flow Mode

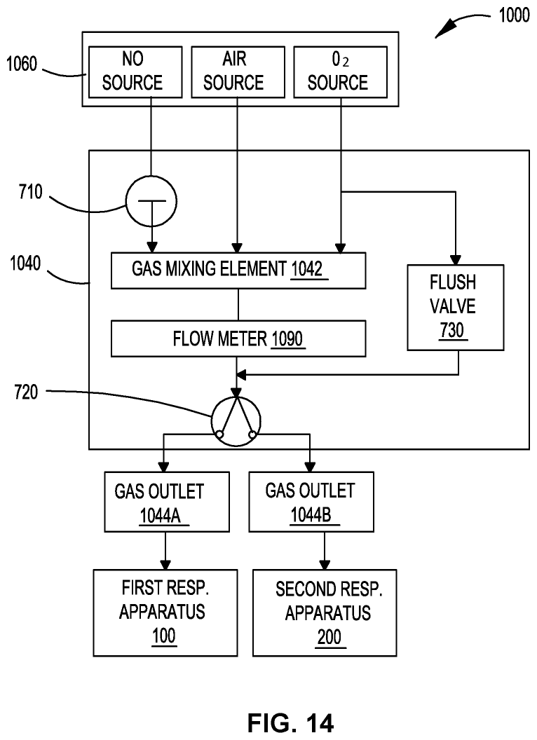

Another aspect of the present disclosure provides a system for delivering breathing gases to a patient which enables delivery of different modes of respiratory support including ventilation, anaesthesia and in some embodiments, high flow respiratory support. FIGS. 15A and 15B are schematic drawings of a system 1300 which has a flow generator 1350 which is adapted to receive fresh air and gases 1310 from the environment and propel them through the system for delivery to the airway 310 of a patient 300 through an inspiratory conduit 110 coupled with a sealing first patient interface 120. A switching actuator such as a button, switch, knob, foot operated switch or pedal, or an electronic interface in operable communication with a system controller, is operable by a user to select a mode of operation of the system 1300 wherein in a first mode (FIG. 15A) breathing gases are delivered in a closed gas flow circuit in which expired gases are returned to the system by expiratory conduit 130 for rebreathing by the patient, and in a second mode (FIG. 151B) in which breathing gases are delivered in an open gas flow circuit without rebreathing.

Ideally, the flow generator 1350 is a blower with variable control which is selectable by a user or a system controller. Ideally the switching actuator includes or is operatively coupled with a switching mechanism 1370 which controls flow of fresh gas into system 1300 according to the mode of operation selected using the switching actuator. In the first mode (FIG. 15A) the switching mechanism 1370 prevents a first flow of fresh breathing gas to the system and permits return of expired gases to the system whereas in the second mode the switching mechanism permits the first flow of fresh breathing gas into the system and prevents return of expired gases to the system. In some embodiments, the switching mechanism 1370 is a gas flow diverter and may, in some arrangements, be a pressure controlled gas flow diverter configured to detect back pressure in the system 1300 and operate in the first mode, when a high back pressure is detected, signalling that a sealed patient interface returning expired gases to the system has been applied to the patient 300.

Typically, the switching mechanism 1370 is located upstream of the flow generator 1350 as shown in FIGS. 15A and 15B. Switching mechanism 1370 is operatively coupled with the flow generator 1350 such that operation of the switching actuator to select the first mode causes operation of the flow generator at a low flow rate such as less than 15 LPM, and selection of the second mode causes operation of the flow generator at a higher flow rate compatible with patient ventilation, and selection of a third mode causes operation of the flow generator at high flow rates of up to 90 LPM.

In some embodiments, operation of system 1300 in the first mode enables delivery of anaesthesia. In such embodiments, system 1300 also receives a supply of oxygen from O₂ source 1060 and vaporized volatile anaesthetic agents such as sevoflurane from vaporizer 150. These gases are mixed in the required proportions and concentrations (as determined by a user or a controller of the system) by gas mixer 1042. The mixed gases are delivered to the patient via inspiratory conduit 110 and sealing first patient interface 120 into the patient's airway 310. Expired gases are returned by the first patient interface 120 to the expiratory conduit 130 and to the system 1300, where CO2 is removed by CO2 absorber 141 and the gases are recycled back to the inspiratory pathway.

In some embodiments, operation of system 1300 in the second mode enables provision of ventilatory support to the patient. This form of support can be delivered by the same first patient interface 120 as in the first mode of operation with the distinction that the switching mechanism 1370 operates to permit flow of fresh air into the system 1300 while preventing recirculation of gases. Thus, gases returned by expiratory conduit 130 may be vented to the environment or an exhaust, or released through vent holes in a patient interface through which respiratory support is delivered. In another form of respiratory support delivered in the second mode, the first patient interface 120 is replaced with a non-sealing patient interface such as a nasal cannula, and the flow rate generated by flow generator is increased to deliver high flows, so that the system 1300 is operated in a second mode delivering high flow respiratory support to the patient 300. Thus, operation of the switching actuator to select the second mode causes operation of the flow generator at a flow rate (e.g. up to 90 LPM) sufficient to deliver high flow respiratory support. In embodiments where switching actuator 1370 is a pressure controlled gas flow diverter, a low back pressure in the system 1300 consistent with connection of second, non-sealing patient interface 220 such as nasal cannula (or disconnection of the expiratory conduit 130 from the system 1300) automatically opens flow of fresh gas into the system while shutting off recirculation of gases. O₂ may also be provided from O₂ source 1060 during operation of the system in the second mode, with the O₂ concentration selected by operation of the switching actuator which may be operatively coupled with the O₂ source or gas mixer 1042, or by manual selection of O₂ concentration at a supply controller provided at the O₂ source. System 1300 is also configured to prevent supply of anaesthesia gases to the system when the second mode is selected. This may be achieved by providing shutting off vaporizer 150 either manually by the user, or by providing a vaporizer switching mechanism that is operatively linked with the switching mechanism 1370 and/or system controller 1010.

In some embodiments, system 1300 includes a pressure relief valve 146 to maintain substantially stable gas pressure in the system when operating in the first mode, which would otherwise increase with addition of oxygen and volatiles to system 1300 in the first mode. An exhalation valve 143 or variable flow constriction may be provided to modulate gas flows in the system 1300 to generate expiratory and inspiratory breathing cycles, particularly when this cannot be achieved by modulating flow through of flow generator 1350. As shown in FIGS. 15A and 151B, in some embodiments system 1300 is configured to receive a supply of anaesthesia gases from the vaporizer 150 located downstream of the flow generator 1350 before inspiratory conduit 110 and the expiratory conduit 130 is couplable with the system 1300 downstream of the flow generator. However, that need not be the case.

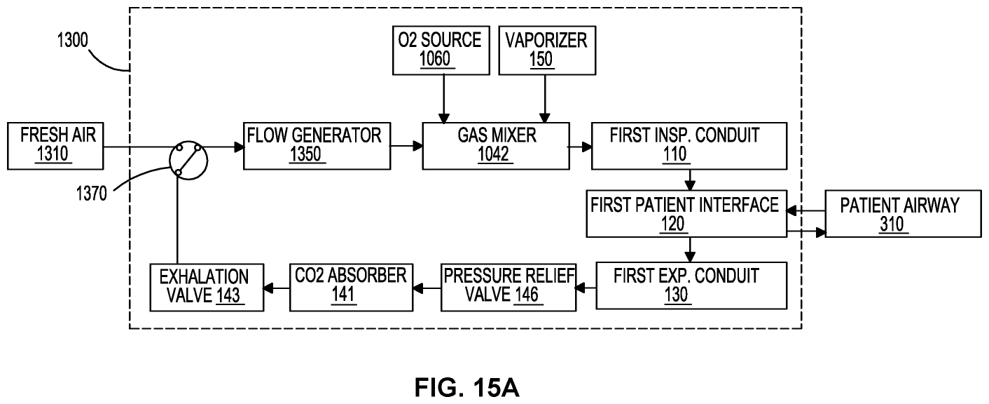

FIGS. 16A to 16E are schematic illustrations showing various alternative embodiments for system 1300. In FIG. 16A vaporizer 150 is located downstream of the flow generator 1350 and switching mechanism 1370 provides a separate gas flow path via a separate inspiratory conduit for delivering high flow respiratory support to a second non-sealing patient interface. An adjustable pressure limiting valve (APL) is provided to release returned gases into the scavenger system (as described in relation to FIG. 1). It is to be understood however that in the various embodiments disclosed, an APL may be represented by any form of relief valve and that an adjustable pressure-limiting valve is just one example. In FIG. 16B vaporizer 150 is provided in the returned gases flow path, upstream of CO2 absorber 141 (although it could be located downstream of CO2 absorber 141). In FIG. 16C vaporizer 150 is connected in parallel with flow generator 1350 and ventilation bag 142 provides for manual ventilation of the patient. In FIG. 16D, vaporizer 150 is connected upstream of the flow generator 1350 which receives and drives gases including anaesthetic agents to the inspiratory conduit 110. In FIG. 16E, system 1300 includes a gas flow reflector 1360 configured to collect anaesthetic gases expired from the patient and return them to the inspiratory gas flow path in a following inhalation phase.

In various embodiments, system 1300 includes a humidifier configured to condition the breathing gas to a pre-determined temperature and/or humidity before delivery to the patient in the second mode. Ideally, the humidifier (not shown) is located downstream of the blower, before the second patient interface 220.

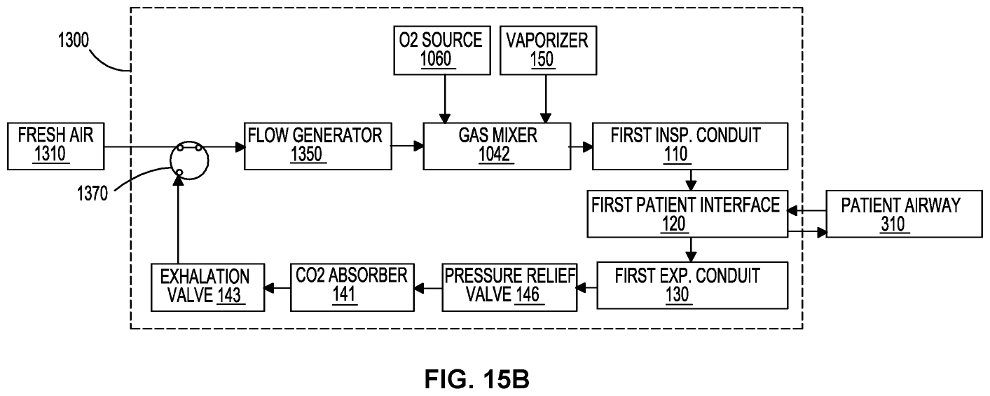

Another system for delivering breathing gases to a patient which enables delivery of different modes of respiratory support including ventilation, anaesthesia and in some embodiments, high flow respiratory support is shown in the schematic illustration of FIG. 17. Here, system 1300 is operable in a first mode and a second mode, wherein the first mode includes a recirculating gases flow between the system and the patient's airway, and the second mode includes a non-recirculating gases flow between the system and the patient's airway. System 1300 includes a first module 1380 including a first set of respiratory components and a second module 1390 including a second set of respiratory components. The second module 1390 is configured to cooperate with the first module 1380 (or vice versa) to switch between the two modes. Thus, the system 1300 is operable in the first mode when the first module 1380 is activated or coupled together with the second module 1390, and the system is operable in the second mode when the first module is inactivated or uncoupled from the second module, such that the second module functions independently of the first module.

In some embodiments, during the first mode of operation of system 1300, gases delivered to the patient 300 in the recirculating gases flow include an anaesthetic agent delivered by vaporizer 150 which vaporizes volatile anaesthetic agents into breathing gas delivered to the patient. Thus, the first set of respiratory components of the first module 1380 may include one or more of: a CO2 absorber 141 configured to treat returned expired gas before recirculating to the patient in the first mode; a pressure limiting valve 146 configured to maintain substantially stable pressure in the system in the first mode; a variable volume (such as a ventilation bag 142 or bellows) for displacement of gases in the first mode. A fresh gas flow from vaporizer 150 replenishes anaesthetic gas delivered to the patient in the first mode.

During the second mode of operation the second module 1390 operates independently of the first module 1380 to deliver e.g. ventilatory support and in some embodiments, high flow respiratory support to the patient 300. Thus, the second set of respiratory components of the second module 1390 may include one or more of a flow source such as a blower 1350 configured to generate gas flows through the second set of respiratory components; an inspiratory conduit 110 and a patient interface configured to direct gases from the non-recirculating gases flow to the patient's airway. When the system 1300 is used in the second mode to deliver ventilatory support, the patient interface may be a sealing patient interface, with expired gases returned to the system via expiratory conduit 130 where they are vented to atmosphere or an exhaust (and not recirculated to the patient). Alternatively expired gases may exit the sealing patient interface through port holes in the interface or the expiratory conduit. When the system 1300 is used in the second mode to deliver high flow respiratory support, the patient interface is ideally a non-sealing second patient interface such as a nasal cannula. A humidifier (not shown) may be provided to condition the breathing gas to a pre-determined temperature and/or humidity before delivery to the patient in the second mode; and a filter upstream of the patient interface may be provided to reduce contamination of the second set of respiratory components upstream of the filter so that they may be reused.

In some embodiments the first module 1380 includes a first gases outlet 1388 and a first gases inlet 1386, and the second module 1390 includes a second gases inlet 1396 and a second gases outlet 1398, wherein the first gases outlet is couplable with the second gases inlet and the first gases inlet is couplable with the second gases outlet for operation of the system 1300 in the first mode, as shown in FIG. 17. Thus, in the first mode of operation the system utilises the same patient breathing circuit, comprising inspiratory conduit 110 and expiratory conduit 130, to deliver breathing gases including anaesthetic agents to the patient. A one way valve 149 may be provided to prevent backflow of gases to the patient. It is to be understood that in use, the patient interface may be switched by a user to ensure that gases delivered in the first mode are via a sealing first patient interface such as a sealing mask or endotracheal tube and gases delivered in the second mode are via a non-sealing second patient interface such as a nasal cannula, or a sealing patient interfaces with expired gases vented or exhausted i.e. not returned to the patient.

Notably, operation of the system 1300 in the first mode permits a first flow of fresh breathing gas 1310 to the system and simultaneously permits return of expired gases to the system due to the coupling between second gases outlet 1398 and first gases inlet 1386. A one way valve 149A may be provided to prevent backflow of returned gases to the fresh gas supply 1310. Meanwhile, operation of the system 1300 in the second mode permits the first flow of fresh breathing gas 1310 into the system independently of the first module 1380 and prevents return of expired gases to the system due to decoupling of second gases outlet 1398 and first gases inlet 1386. Additionally, operation in the second mode prevents release of anaesthetic agents from the system e.g. by provision of a shut off valve in first gas outlet 1388 and/or by switching off vaporizer 150 and/or providing a bypass around vaporizer 150.

### Switching Provided in The Breathing Circuit

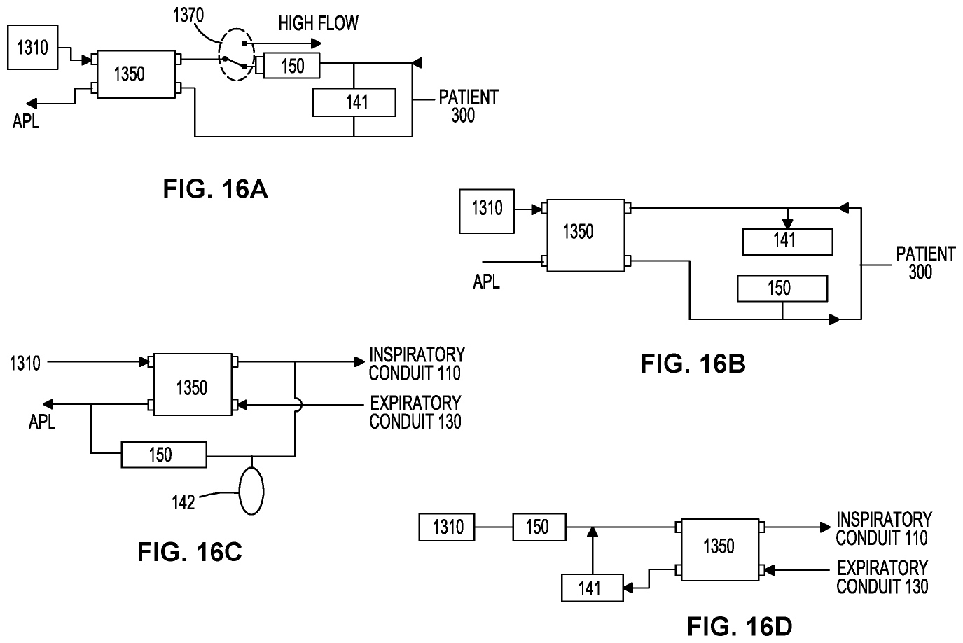

Another aspect of the present disclosure relates to switching at the patient end of a breathing circuit delivering breathing gases to a patient 300 in a first mode or a second mode. In one embodiment, a system for delivering breathing gases to a patient, includes a flow modulator 250 configured to generate gas flows through the system in a gas delivery circuit and a switching mechanism forming part of the gas delivery circuit. FIG. 18A is a perspective sketch of a switching mechanism 900 which is configured to switch between gas flow paths in the gas delivery circuit according to selection of a first mode of operation in which inspiratory gas flow path 910 is in fluid communication with a first patient interface 120, or a second mode of operation in which inspiratory gas flow path 910 is in fluid communication with a second patient interface 220.

Typically, the first patient interface 120 forms a substantially sealing interface with the patient's airway, and receives expiratory gas from the patient. Thus in the first mode, expiratory pathway 930 returns expired gases to the system, specifically to a ventilator or anaesthesia machine of the system where the expired gases are treated e.g. by filtering and released to atmosphere, or recirculated in the anaesthesia machine rebreathing system. Typically, the second patient interface 220 forms a non-sealing interface with the patient's airway and is e.g. a nasal cannula which permits release of expired gases to the environment, around the non-sealing nasal prongs of the cannula.

In one embodiment illustrated in FIG. 18A, switching mechanism 900 includes an ‘x-piece’ connector 950 and actuator 940 in the form of a rotary switch which is operable by a user to switch between gas flow paths in the gas delivery circuit. The actuator may take any suitable form such as e.g. a knob, switch, lever or the like. Also shown in FIG. 18A is an optional anaesthetic reflector 960. FIG. 18A is a top sectional illustration of the switching mechanism 900 of FIG. 18A in the first mode, in which breathing gas in inspiratory flow path 910 is directed to first patient interface 120 and expired gases are returned through a common conduit to the switching mechanism and returned to the system through expiratory gas flow path 930. FIG. 18C is a top sectional illustration of the switching mechanism 900 of FIG. 18A in the second mode, in which breathing gas in inspiratory flow path 910 is directed to second, non-sealing patient interface 220. No gases are returned in this mode.

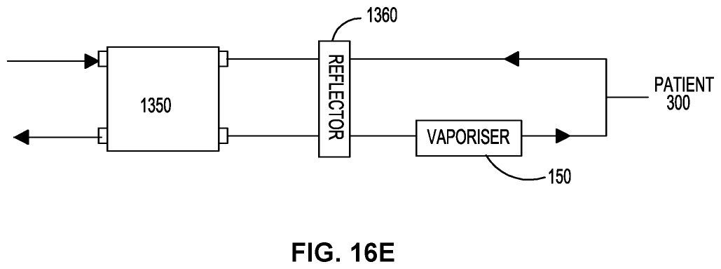

In some embodiments, the system is configured to receive a user input to select the first mode of operation in which the flow source 1350 generates a low flow rate below a pre-determined flow rate, or the second mode of operation in which the flow source generates a high flow rate at or above the pre-determined flow rate, and wherein the switching mechanism 900 operates responsive to flow rate of gas in the inspiratory gas flow path as generated by the flow source. A bi-stable switch 948 as depicted in FIGS. 19A and 19B could be used in such an embodiment. When flow through the ‘x-piece’ exceeds a threshold flow rate e.g. 15 LPM or e.g. corresponding to delivery of high flow respiratory support, flow is delivered to the second patient interface 220 (FIG. 19B). Below the threshold flow rate, flow is delivered to the first patient interface 110 and expired gas is returned through expiratory flow path 930 (FIG. 19A). It is to be understood, however, that the switching mechanism may include any suitable mechanism including but not limited to one or more of a gas flow diverter, pneumatic switch, rotary switch, lever, flap or valve or the like.

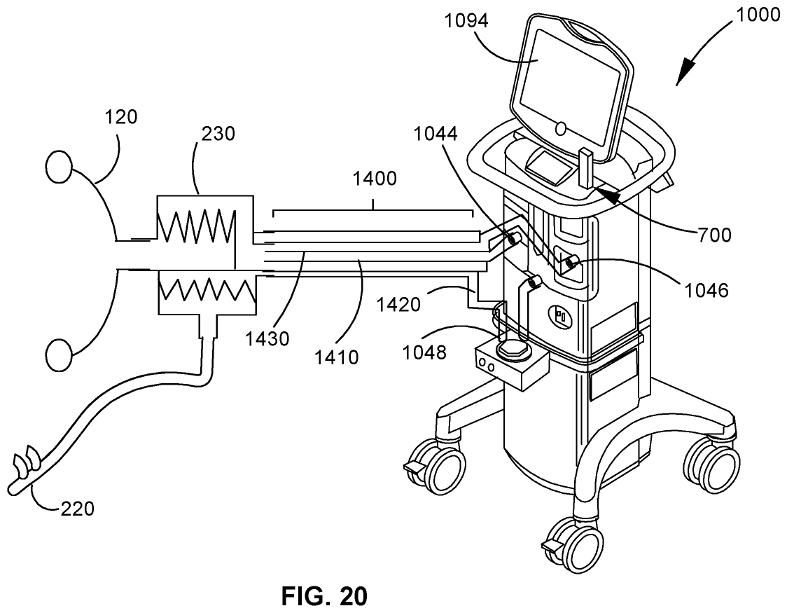

In some embodiments, a user controlled actuator 1440 operates a flow diverter controlling flow of breathing gases toward a patient. An example is shown schematically in FIGS. 23A and 23B. In some embodiments, actuator 1440 is operatively coupled with the system 1000 so that operation of the actuator by a user to switch flow delivery between patient interfaces causes control of system 1000 to switch to the mode of operation in which breathing gases are delivered. For example, In FIG. 23A, actuator 1440 is in a first position and directs breathing gases from first inspiratory conduit 1410 to the first patient interface 120 which returns expired gases via expiratory conduit 1430. This is consistent with a first mode of operation which, as disclosed herein provides for delivery of breathing gases including an anaesthetic agent, and return of expired gases for treatment by rebreathing components. In FIG. 23B, actuator is in a second position and directs breathing gases from inspiratory conduit 1410 to the second patient interface 220 which is a non-sealing interface such as a nasal cannula. This requires the system controller 1010 to direct breathing gases into inspiratory conduit 1410 consistent with the second mode of operation (i.e. high flow with no anaesthetic agents). Expired gases are exhaled into the environment. This is consistent with a second mode of operation which, as disclosed herein provides for delivery of high flow respiratory support.

In some embodiments, the system utilising the switching mechanism 900 includes one or more sensors configured to monitor one or more characteristics of gas in the gas delivery circuit, and controls operation of the flow modulator 250 based on said one or more monitored characteristics. These characteristics may indicate one or more of e.g. flow, pressure and CO₂. The system controls operation of the flow modulator 250 to generate low flows (e.g. less than 15 LPM) when the one or more sensors indicate breathing gas flow is to the first patient interface 110, and to generate high flows when the one or more sensors indicate breathing gas flow is to the second patient interface 220 using various techniques described herein.

As one of skill would understand when considering this disclosure as a whole, the system utilising the switching mechanism 900 or other switching concepts disclosed herein may include one or more components commonly found in anaesthesia machines, such as a CO₂ absorber configured to treat returned expired gas before recirculating to the patient in the first mode, a pressure limiting valve configured to maintain substantially stable pressure in the system in the first mode, a variable volume (e.g. ventilation bag or bellows) for displacement of gases in the first mode, a fresh gas flow for replenishing anaesthetic gas delivered to the patient in the first mode, and a vaporizer for vaporizing volatile anaesthetic agents into breathing gas delivered to the patient in the first mode. Additionally, such a system may include one or more features of a high flow respiratory support system, such as a flow modulator configured to provide high flows through the system and a humidifier configured to condition the breathing gas to a pre-determined temperature and/or humidity before delivery to the patient in the second mode.

While switching mechanism 900 has been described in the context of a component of a system 1000, it is to be understood that the switching mechanism 900 may be supplied as a component separate from such a system. In this sense, the switching mechanism 900 may be regarded as a breathing gas connector having an inlet port 911 couplable with a gas flow conduit 910 receiving breathing gases from the respiratory support system, a first outlet port 921 couplable with a first gas flow path delivering breathing gas to the patient through a first patient interface 120, a second outlet port 922 couplable with a second gas flow path delivering breathing gas to the patient through a second patient interface 220 and a switching mechanism 940 operable to switch the connector between a first mode of operation the in which the connector directs gas from the inlet port to the first outlet port and a second mode of operation in which the connector directs gas from the inlet port to the second outlet port.

An expiratory gases port 931 is couplable with an expiratory gas conduit, wherein in the first mode, the switching mechanism directs expired gases in the first gas flow path to the expiratory gases conduit. The switching mechanism may be operatively couplable with a controller 1010 of the respiratory support system, wherein operation of the connector switching mechanism 940 may select the first mode of operation of the second mode of operation. The first mode of operation causes the controller to operate the respiratory support system in a first mode in which breathing gases including an anaesthetic agent are delivered to a gas flow conduit 910 coupled with the connector. The second mode of operation causes the controller to operate the respiratory support system in a second mode in which breathing gases are delivered at a pre-determined flow rate to a gas flow conduit 910 coupled with the connector. In some embodiments, connector switching mechanism 940 is operatively couplable with a controller of the respiratory support system such that operation of the connector switching mechanism to select the second mode of operation causes the controller to prevent flow of anaesthetic agents in the breathing gas.

A sensor may be provided to detect one or more characteristics of gas at the first outlet port 921 or the second outlet port 922, said characteristics being used to determine if the connector is coupled to the patient's airway by a first (sealing) patient interface 120 or a second (non-sealing) patient interface 220, the sensor providing input to a controller of the respiratory support system which automatically selects a corresponding mode of operation of the respiratory support system. The one or more characteristics include but are not limited to gas pressure, CO₂ concentration; and gas flow rate. A sensor wire locatable inside a conduit providing a gas flow path between the sensor and the respiratory support system controller.

In another aspect, a switching mechanism may be provided by simultaneous use of the first and second patient interfaces 120, 220. Thus when the first and second patient interfaces are applied to the patient simultaneously the first mode is selected, and when only the second patient interface is applied to the patient the second mode of delivery is selected. In one arrangement, the first patient interface is a mask is capable of sealing over the second patient interface being a nasal cannula without occluding flow through the cannula. Inspiratory flow is delivered via the cannula with expiratory gases returned via the face mask. This arrangement can be deployed to deliver anaesthesia in a closed system utilising the cannula to deliver anaesthetic agents to the patient and the mask to return expired gases. Pressure sensors may be used to determine when the mask is applied to the patient and sealed over the cannula, such that when a target pressure is detected at the mask (e.g. inside the mask or within the mask cuff), then the system responds by initiating delivery of anaesthetic agent through the nasal cannula. Commonly owned patent publication WO2015/145390 discloses a mask suitable in this context and is hereby incorporated herein by this reference.

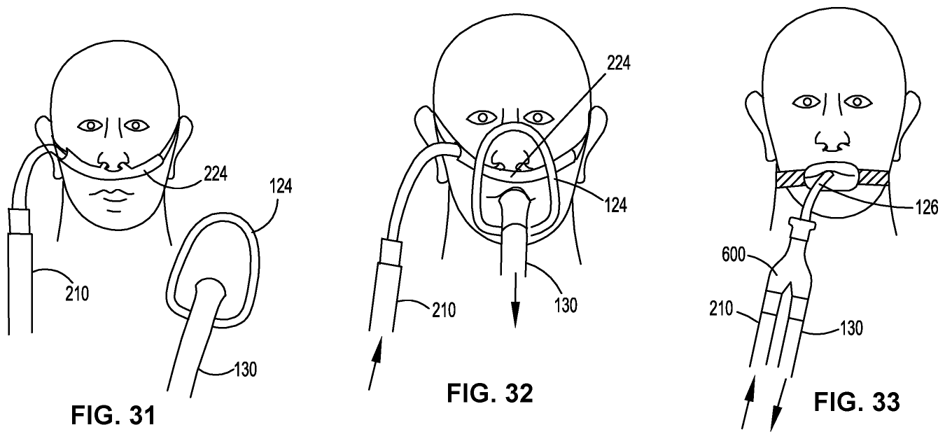

FIGS. 31 to 33 illustrate one example of how switching between modes of use of a system for delivering breathing gases to a patient may be provided by simultaneous or separate use of the first and second patient interfaces. The system includes a flow source configured to provide gas flows through the system in a gas delivery circuit having an inspiratory gas flow path 210 and an expiratory gas flow path 130. In the first mode, the system is operable to deliver the breathing gases to the patient via the inspiratory gas flow path 210 and a first patient interface in fluid communication with the inspiratory flow path, and to deliver expiratory gases from the patient via the expiratory gas flow path 130 and a second patient interface in fluid communication with the expiratory gas flow path, the breathing gases including a first flow parameter. In the second mode, the system is operable to deliver the breathing gases to the patient via the inspiratory gas flow path 210 and a third patient interface, the breathing gases including a second flow parameter. The first and second flow parameters include or correspond to a first and second flow rate respectively. In some embodiments, the first flow rate is less than 15 L/min and the second flow rate is greater than 15 L/min. In some embodiments, the second flow rate is in the range of between about 20 L/min and about 90 L/min, optionally between about 40 L/min and about 70 L/min. It is to be understood, however, that the first flow parameter may alternatively or additionally include a pressure and/or volume parameter.

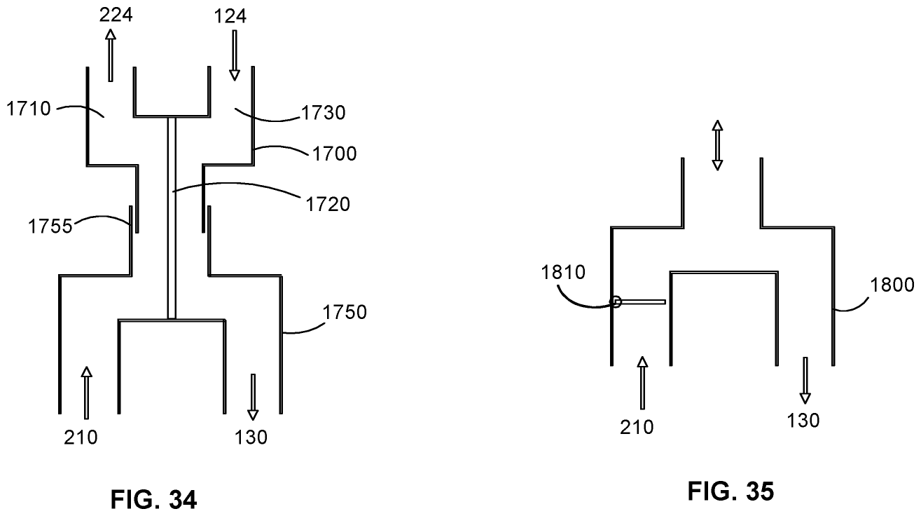

As shown in FIG. 31, the first patient interface is a non-sealing patient interface shown as nasal cannula 224 which is in fluid communication with inspiratory gas flow path 210, and the second patient interface is a sealing patient interface shown as mask 124. In FIG. 32, the nasal cannula 224 and mask 124 are applied to the patient simultaneously such that the system is operable in the first mode providing anaesthetic ventilation, with the mask 124 configured to seal over the nasal cannula 224 and with the patient. In this arrangement, breathing gases are delivered to the patient via the inspiratory conduit providing inspiratory gas flow path 210 and nasal cannula 224, and expiratory gases from the patient are delivered from the patient via the mask 124 and the expiratory conduit providing expiratory gas flow path 130. In FIG. 31, only the nasal cannula 224 is applied to the patient for delivery of breathing gases to the patient's airway in the second mode providing high flow respiratory support. In FIG. 31, the first and third patient interfaces are the same and are the nasal cannula 224. In some embodiments, the expiratory flow path 130 is not required or is inoperable in the second mode. In an alternative embodiment, the breathing gases are delivered to the patient via gas flow path 130 and the mask 124, and expiratory gases from the patient are delivered from the patient via the nasal cannula and gas flow path 210. In such an embodiment, the gas flow path 130 is an inspiratory gas flow path and the gas flow path 210 is an expiratory gas flow path. In some embodiments, the expiratory gases are returned to the system.

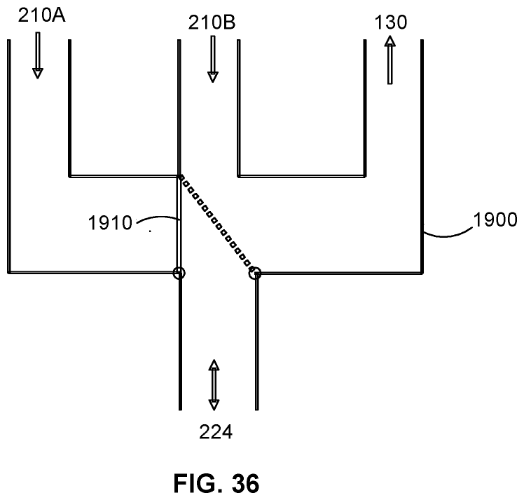

FIG. 33 provides an arrangement for operation of the system in a third mode. In the third mode, anaesthetic ventilation is delivered by an endotracheal tube 126. Here, the inspiratory conduit providing inspiratory gas flow path 210 has been decoupled from nasal cannula 224 and coupled with an inlet of coupling 600. Similarly, the expiratory conduit providing expiratory gas flow path 130 has been decoupled from mask 124 and coupled with an outlet of coupling 600. Patient end of coupling 600 has been connected with a fourth patient interface shown as an endotracheal tube 126. In some embodiments the endotracheal tube 126 may be a laryngeal mask or a tracheostomy interface. In the third mode, the breathing gases may be delivered to the patient including a third flow parameter which may include one or more of a flow rate, pressure or volume parameter.

An advantage of mode switching according to the examples described in relation to FIGS. 31 to 33 is that a single inspiratory conduit and a single expiratory conduit can be used to provide several modes of respiratory support to the patient. This reduces cost and complexity of breathing circuits utilised to treat the patient.

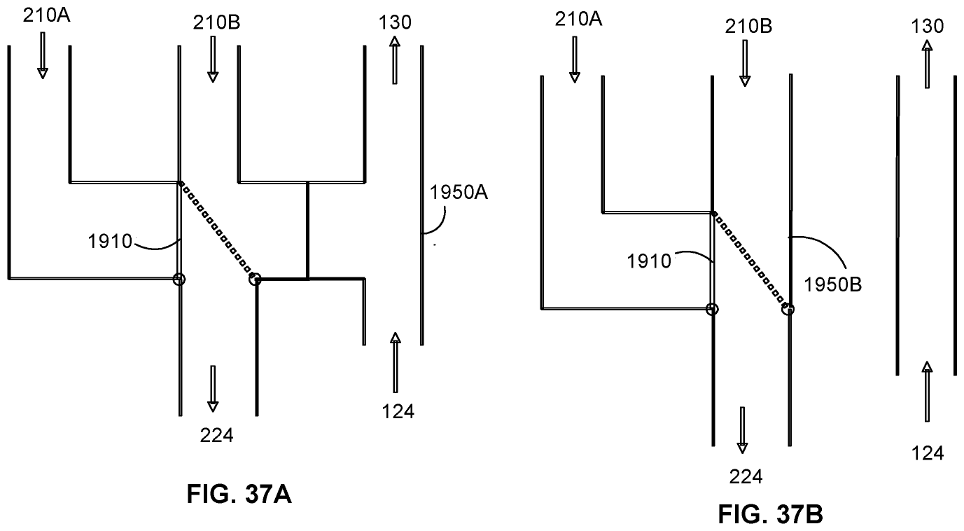

FIG. 34 is a schematic illustration of a novel connector 1700 that may be used to facilitate interchanging of components for delivery of respiratory support in the first and second modes as described in relation to the embodiments of FIGS. 31 to 33. The connector 1700 is configured to couple with a standard wye-piece connector 1750. In normal use, wye-piece connector 1750 is configured to couple at port 1755 with a conduit providing a flow of the respiratory gas to a mask 124 of the type shown in FIGS. 31 and 32. Wye-piece connector 1750 receives a flow of respiratory gas from an inspiratory flow path 210 and provides an outflow pathway for expired gases from the patient via expiratory gas flow path 130. When used consistent with FIG. 32, the mask 124 form a sealed interface with the patient's airway for delivery of breathing gases which may include anaesthetic agents, while returning expired gases to the respiratory apparatus via the expiratory gas flow path 130.

To facilitate interoperability between different patient interfaces, the novel connector 1700 may be used, which provides a wall 1720 configured to protrude into the wye-piece connector 1750. When connected, the wall 1720 separates the flows in the inspiratory and expiratory flow paths 210, 130 into separate limbs 1710 and 1730. In use, connector 1700 is configured to couple first limb 1710 with a conduit attached to a nasal cannula 224 and is configured to couple second limb 1730 with a conduit attached to a face mask 124. Owing to the separating wall 1720, inspiratory flow to the cannula 224 (see FIGS. 31, 32) remains separate from the expiratory flow received from the mask 124 (when used in the configuration of FIG. 32). To switch swiftly to the apparatus required to deliver support in the third mode, the connector 1700 can be removed from the wye-piece connector 1750 which instead is coupled with an endotracheal tube 126. Advantageously, connector 1700 can be used to couple and decouple the wye-piece connector 1750 from the nasal cannula 224 and the mask 124 simultaneously and with fewer disconnections/reconnections of parts for swift connection of the endotracheal tube 126 and less scope for error. When switching back to the first mode of support with the mask 124 configured to seal over the nasal cannula 224 (as per FIG. 32), the endotracheal tube 126 is decoupled from wye-piece connector 1750 and the connector 1700 is reconnected, simultaneously connecting the cannula 224 and mask 124.

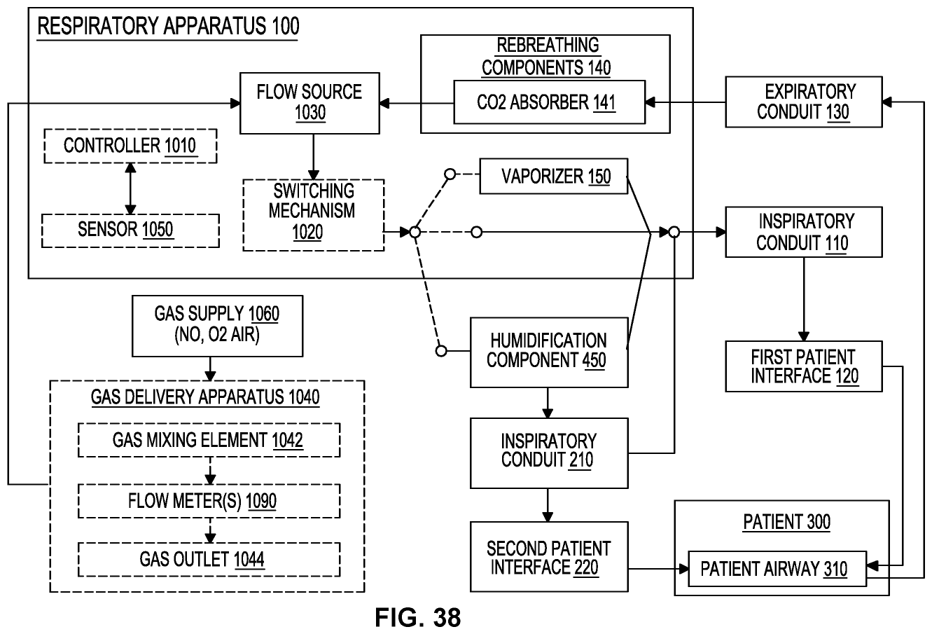

FIG. 35 is a schematic illustration of another connector 1800 provided for use in the arrangement illustrated in FIG. 32 in lieu of the standard wye-piece connector 1750. Using connector 1800, the mask 124 is used to remove expired gas via expiratory gas flow path 130. For operation in the second mode (FIG. 32), connector 1800 may remain in place connected between the mask and the expiratory gas flow path 130 but decoupled from the inspiratory gas flow path 210 which is instead couple with the nasal cannula 224. One way valve 1810 is biased for flow in inspiratory gas flow path 210 toward the mask 124, operating to prevent expiratory gases from the mask from exiting to atmosphere since there is no inspiratory gas conduit attached to the connector 1800 in this mode of operation. To operate in the third mode (FIG. 33), the inspiratory conduit can be reconnected to the connector and the mask 124 can be decoupled and replaced with connection of the endotracheal tube 126. An advantage of this arrangement is that connector 1800 may be used in delivery of both modes of respiratory support shown in FIGS. 32 and 33 while avoiding the need to disconnect and reconnect all the conduits required to deliver and remove gases as is the case with a standard wye-piece connector.

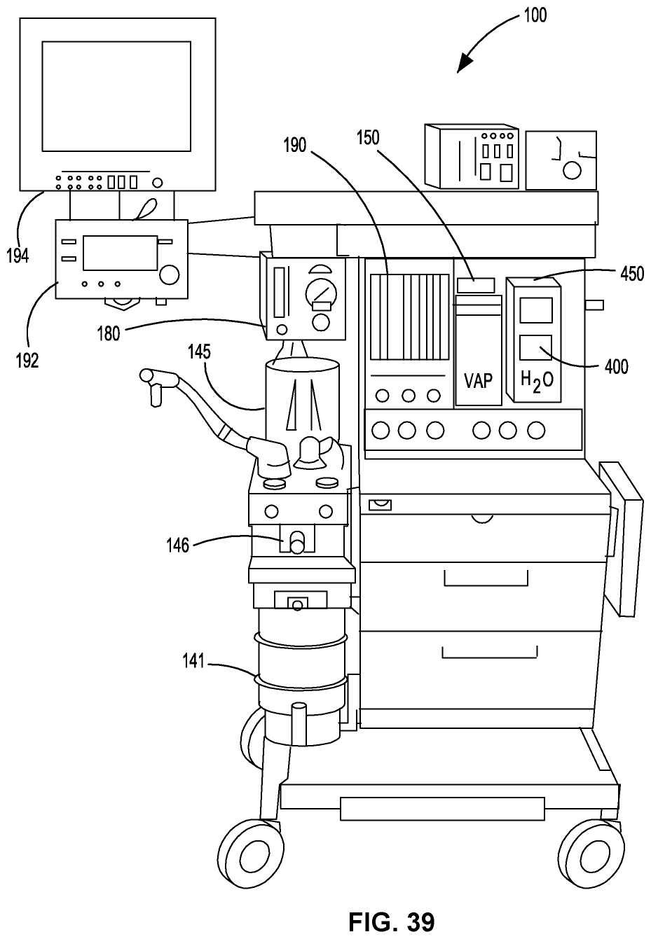

FIG. 36 is a schematic illustration of another novel connector 1900 configured for use in an embodiment where a nasal cannula 224 is used to deliver different modes of respiratory support. Connector 1900 is couplable with three conduits providing fluid communication with each of a first inspiratory gas flow path 210A configured to provide a flow of gases for delivery of nasal high flow respiratory support; a second inspiratory gas flow path 210B configured to provide a flow of gases for delivery of anaesthetic ventilation, and an expiratory gas flow path for exit of expired gases from the patient. A switch 1910 (such as a switching valve, solenoid or the like) is provided to alter internal flow paths of the connector 1900 when a different mode of support is selected. When the nasal cannula 224 is used for delivery of anaesthetic ventilation, switch 1910 is in the position shown in solid line, permitting delivery of respiratory gases (including anaesthetic agents) from the inspiratory gas flow path 210B, and providing a pathway for expired gases, 130. During rebreathing, a bag mask can be applied over the nasal cannula 124 with sufficient pressure applied (e.g. by a clinical attendant) that the cannula can provide both the inspiratory and expiratory flow paths. In high flow respiratory support, switch 1910 is in the position shown in broken lines. Switch 1910 may be operatively linked with other switching means in the system which are operated by a user (or a system controller) to determine the mode of support that is to be delivered.

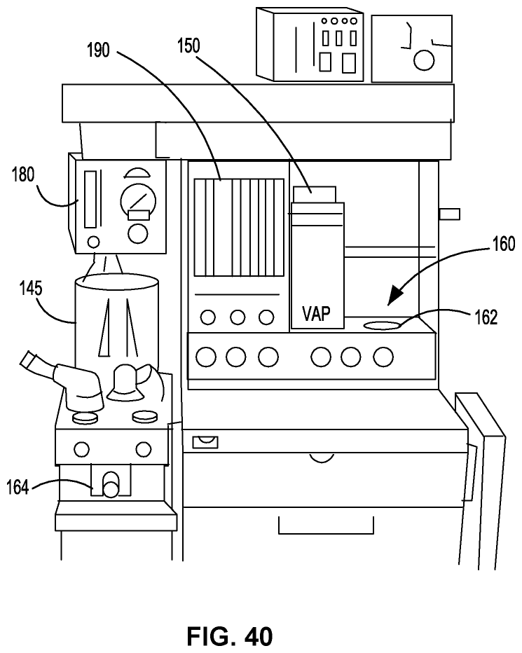

FIGS. 37A-B are schematic illustrations of a connector 1950A, B which is a variation on connector 1900 from FIG. 36, wherein there is provision for connection with both a nasal cannula 224 and a mask 124. This connector 1950A,B provides for delivery of nasal high flow when switch 1910 is in the broken line position. When switch 1910 is in the solid line position and anaesthetic ventilation may be provided by cannula 224 with expiratory gases removed by application of face mask 124, with the apparatus arranged as shown in FIG. 32. FIG. 37A shows connector 1950A with all flow paths in a unitary connector piece. FIG. 37B shows connector 1950B comprising the inspiratory flow paths with connection for the nasal cannula 124, with a separate conduit used to provide expiratory gas flow path 130 attached to mask 124.

Typically, a system 1000 with which the patient interfaces shown in FIGS. 31-33 are used may include a controller 1010 in communication with the flow modulator 250, and one or more of a sensor or an input interface in communication with the controller to provide an input to the controller to control the flow modulator to provide the flow of breathing gases in the first or second mode. The sensor and/or input interface may also be configured to provide an input to the controller to control the flow modulator to provide flow of breathing gases in the third mode.

The system may also include a humidifier 420, wherein the breathing gases are heated and/or humidified by the humidifier in the second mode prior to being delivered to the patient. In some embodiments, the breathing gases in the first and/or third modes may be heated and/or humidified by the humidifier 420. Ideally expiratory gases from the patient are returned to the inspiratory gas flow path 210 in the first mode and the third mode. A CO₂ absorber 141 may be provided to remove CO₂ from the expiratory gases before returning the expiratory gases to the inspiratory gas flow path 210.

### Tri-Lumen Tube Assembly

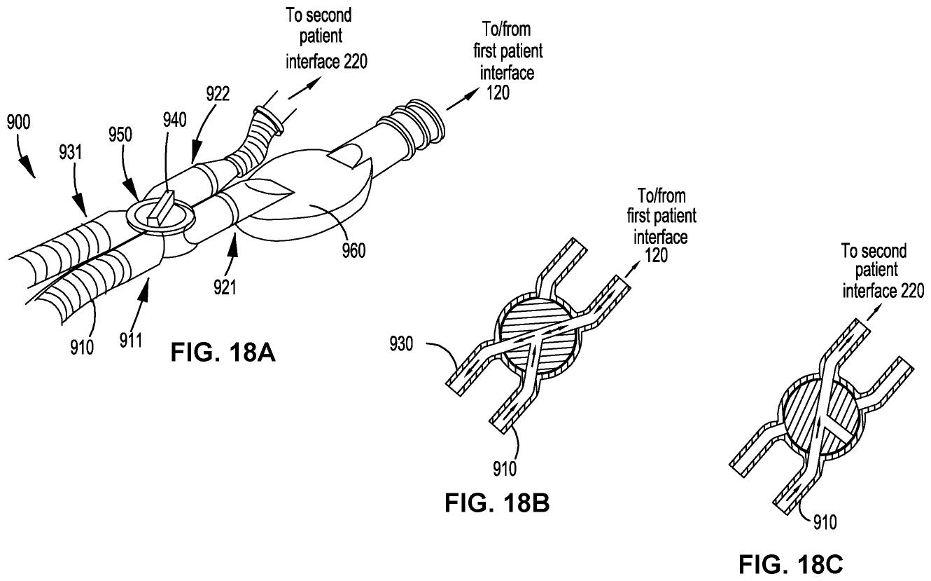

Another aspect of the present disclosure provides a multi-lumen assembly 1400 for use with a respiratory support system 1000 as depicted schematically in FIG. 20. The multi-lumen assembly 1400 has a plurality of conduits as shown in FIG. 21. A first inspiratory conduit 1410 has a first conduit inflow end couplable with a first gas outlet 1044 of the respiratory support system and configured to deliver breathing gases including an anaesthetic agent to the patient. The first inspiratory conduit 1410 has a first conduit outflow end couplable with a first patient interface 120 configured to sealingly engage with and direct flow into an airway of the patient. In the embodiment shown, first patient interface 120 is schematically depicted as a sealing mask however it is to be understood that the first patient interface may be an endotracheal tube, LMA or the like.

A second inspiratory conduit 1420 has a second conduit inflow end couplable with a second gas outlet 1048 of the respiratory support system and is configured to deliver high flow breathing gases to the patient at a flow rate between 20 L/min and 90 L/min. The second inspiratory conduit 1420 has a second conduit outflow end couplable e.g. via a 3 way safety connector, with a second patient interface 220 which is configured to direct flow into an airway of the patient and may be a non-sealing interface such as a nasal cannula.

An expiratory conduit 1430 has an expiratory conduit outflow end couplable with an expired gas inlet 1046 of the respiratory support system. The expiratory conduit 1430 is configured to return expired gases from the patient to the respiratory support system.

In some embodiments, the multi-lumen assembly 1400 includes or is operable with a connector portion 1415 wherein the outflow end of the first inspiratory conduit 1410 and an inflow end of the expiratory conduit 1430 form a common gas flow pathway 1416. A schematic illustration is provided in FIG. 21. Common gas flow pathway 1416 is defined by a single gas exchange conduit 1417 which is couplable with first patient interface 120. Ideally, the connector portion 1415 includes a taper, such as a 22 mm taper, to reduce the overall cross section of the assembly in a region of the single gas exchange conduit 1417. In some embodiments, the connector portion is configured to orient the second conduit outflow end of second inspiratory conduit 1420 so that it diverges from the single gas exchange conduit. The connector portion is then couplable e.g. by a 3 way safety connector, with a second patient interface 220 for delivery of high flow respiratory support.

### Retaining Mechanism

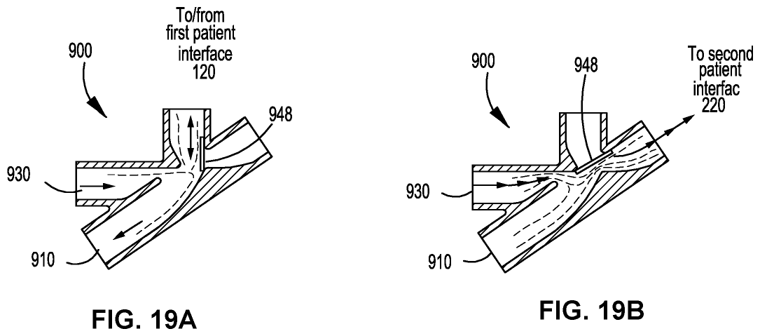

In some embodiments, at least a length of the plurality of conduits is arranged co-axially, with the outer wall of an inner lumen defining the inner wall of the next lumen, as shown schematically in FIG. 21. In other embodiments, at least a length of the plurality of conduits is arranged in parallel and the multi-lumen assembly 1400 includes a mechanism to retain at least a length of the plurality of conduits in a group such as in a strip where the conduits are arranged side by side (FIG. 22A) or in a bundle (FIGS. 22B and 22C). In some embodiments, the plurality of conduits are configured to plug into three respective “slots” of the respiratory support system 1000 to deliver anaesthesia and/or ventilation, and high flow respiratory support, depending on the selected mode of operation of the system. A patient end adapter can be used to plug first and second patient interfaces into a single connector (e.g. connector 1500), or the conduits can be separated along part of the assembly to move the patient ends independently.

In some embodiments, the retaining mechanism includes a webbing 1452 arranged between at least pairs of the plurality of conduits at intervals or continuously along at least a length of the plurality of conduits which are arranged in a strip. The webbing (or webbing portions)1452 may be frangible to facilitate separation of at least a length of one or more of the plurality of conduits from the group e.g. to separate the second inspiratory conduit 1420 which is coupled with a second patient interface 220, whereas the first inspiratory conduit and expiratory conduits are coupled with a single first patient interface 120.

Alternatively or additionally, the retaining mechanism may include a sheath (or skin) 1454 applied around the plurality of conduits as illustrated in FIG. 22B. Ideally, part of sheath 1454 is removable from a length of the multi lumen assembly, e.g. to separate the second inspiratory conduit 1420. The sheath may be formed from any suitable material such as a plastic extrusion or wrap which may have a continuous sheet form, be woven or an expandable “netting” which can be stretched over the plurality of conduits. One benefit of a sheath 1454 formed of a smooth plastic or similar coating is that it is readily cleaned by wiping and has a neat appearance. Advantageously, the patient interfaces can be swapped out, particularly when used in conjunction with filters 230 which may be assembled together with, or incorporated as part of, the patient interface being used. This enables the tri-lumen tube assembly 1400 to be re-used for different patients. Alternatively or additionally, a filter 230 may be incorporated into our couple with the tri-lumen tube assembly 1400 such that a single filter is able to be used with different interchangeable patient interfaces. Additionally it has the capacity to also carry a sensor wire or gas sampling line 227 which may be utilised to transmit sampled expired gas or sensor signals from a sensor located at a patient interface (such as a second patient interface in the form of a nasal cannula) to a controller 1010 of the respiratory support system 1000 which can be used to control switching between modes of operation of the system, as described herein.

Alternatively or additionally, the retaining mechanism may include one or more retainers or clips 1456 configured to retain two or more conduits in the plurality of conduits in a group. An example of a retainer/clip 1456 is illustrated in FIG. 22C and has slots for retaining 3 conduits in a group or bundle. It is to be understood that several retainers 1456 could be used to retain the bundle of conduits together along a desired length. In some embodiments the retainer/clip 1456 provides slots for accommodating 2, 3, 4 or more conduits and may also have slots for retaining e.g. sensor wires or other elongate members. In some embodiments, retainers 1456 are slidable along a length of one of more of the conduits in the plurality of conduits. An advantage of retainers 1456 is that their location on the multi-lumen assembly 1400 can be changed as required, not all slots need to be used, and they are capable of being cleaned and re-used. They may also be used to re-join conduits that have been separated when a web or sheath has previously been provided.

In some embodiments, the multi-lumen assembly 1400 includes or is operable with a flow switching mechanism operable to direct flow of breathing gas into the first inspiratory conduit 1410 or into the second inspiratory conduit 1420. The switching mechanism is operable by a user to select a mode of support which in turn determines the flow of breathing gas into the first or second inspiratory conduit. The switching mechanism is associated or operably coupled with the respiratory support system 1000 and may take any suitable form such as a button, switch, knob, foot operated switch or pedal or other actuator operable by the user. In the embodiment illustrated, switching mechanism is shown schematically as a switch lever 700.

In some embodiments the switching mechanism is operatively linked with a respiratory support system controller 1010 by electronic means enabling the user to switch modes by use of a touch screen or other electronic interface. In some embodiments, the switching mechanism may be coupled directly with a gas flow diverter controlling flows to the first gas outlet 1044 and second gas outlet 1048 of the system. Alternatively, an electronic switching mechanism which has functional control over valves and/or gas flow diverters or other means used to control flow of gases into the first and second inspiratory conduits 1410, 1420 provides scope for flexibility in the location of the switching mechanism, as well as the potential for central control over other components of the respiratory support system 1000 such as vaporizers, gas sources, gas mixers, flow modulators and the like.

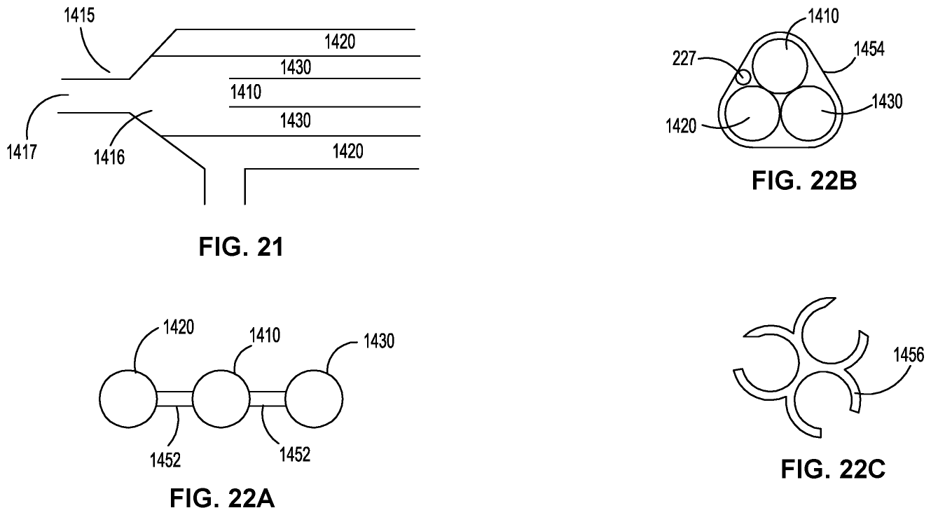

FIG. 24 shows an example of a patient end connector 1500 which is couplable with a tri-lumen assembly 1400 via connector coupling 1550. In some embodiments, the patient end connector 1500 includes one or more switching elements 1570 which are operable by a user to switch the connector between a first mode of operation in which the connector directs breathing gas to the first patient interface via first coupling 1510 and directs expired gases from the patient to a the expiratory flow path via expiratory coupling 130, and a second mode of operation in which the connector directs breathing gas to the second patient interface via second coupling 1520. Thus, switching elements 1570 are in operable communication with a controller 1010 of the respiratory support system 1000 to communicate the mode of selection so that the gas delivery system can be configured accordingly. Alternatively/additionally, controller 1500 may receive control signals from system controller 1010 to redirect flows inside the controller e.g. by activating valves and/or gas flow diverters, to direct breathing gas to the first or second patient interface.

In various embodiments of the multi-lumen assembly 1400 and actuators and connectors that are used with or form part of the multi-lumen assembly, a gas sampling conduit 227 (FIG. 22B) may be provided for monitoring one or more characteristics of gas, such as CO2. These characteristics may be used by a respiratory support system controller to determine if the first or second patient interface is attached to the connector and is applied to the patient's airway.

System Switching with a CO2 Trigger

Another aspect of the present disclosure relates to CO₂ sensing as a means for providing mode switching in a system for delivering breathing gases to a patient. This aspect will be described in the context of a system for providing respiratory support 1000 as described herein, which is operable to deliver breathing gases in a first mode through a first patient interface 120, and in a second mode through a second patient interface 220, The system includes one or more CO₂ sensors configured to detect CO₂ in expired gas from the patient. The system controller 1010 receives an input from the one or more CO₂ sensors and operates the system switching mechanism according to the detected CO₂, selecting the first mode when the detected CO₂ indicates that the first patient interface is connected to the patient, and selecting the second mode when the detected CO₂ indicates that the second patient interface is connected to the patient.

In some embodiments, system controller 1010 switches control when it determines there to have been a change in detected CO₂ concentration. However, detection of a change need not be required since comparison with a reference value (e.g. corresponding to typical ambient CO₂ levels) may be sufficient for the controller to determine if a first patient interface 120 or a second patient interface 220 is attached to the patient. Detection of the patient circuit (and patient interface) connected to the patient enables the system controller 1010 to automatically select the correct mode of operation to deliver the required respiratory support to the patient. For instance, in the first mode breathing gases are delivered to the patient's airway through the first patient interface which may be a sealing interface, and expired gases are returned to the system by an expiratory gas flow path. Expired gases may be redirected to atmosphere or an exhaust, e.g. when breathing gases are delivered to the patient to provide ventilatory support in the first mode. Alternatively, the first mode may be a rebreathing first mode in which expired gases returned to the system are recirculated via a rebreathing system as disclosed herein, for delivery to the patient via the first patient interface. In a rebreathing first mode the breathing gases may include one or more volatile anaesthetic agents vaporised into the breathing gas by a vaporizer as disclosed herein. In the second mode, breathing gas may be delivered at a high flow rate of at least 20 LPM (for adults) and up to about 90 LPM and these gases may be delivered to the patient through a second patient interface 220 which may be a non-sealing interface such as nasal cannula 224.

The system may include a CO₂ sensor associated with a first breathing circuit for delivery of breathing gases in the first mode and/or a second breathing circuit for delivery of breathing gases in the second mode, and the controller 1010 may determine the breathing circuit containing highest concentration of CO₂ to be the breathing circuit that is attached to the patient and controls delivery of gases to the determined breathing circuit according to the relevant mode of operation.

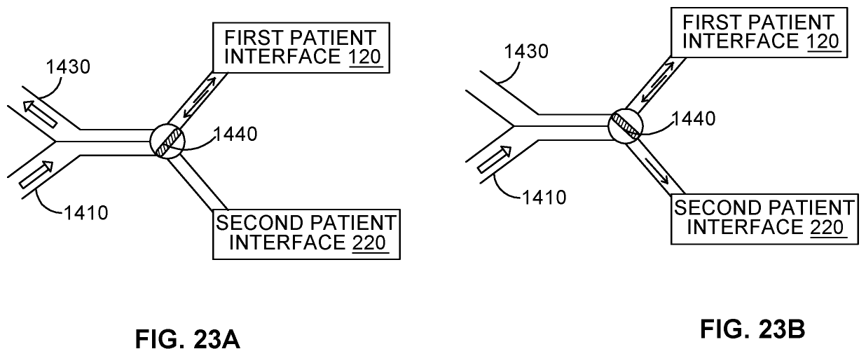

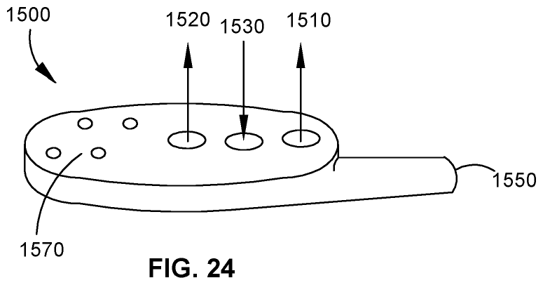

Typically, when the one or more CO₂ sensors detect CO₂ in expired gases in the expiratory gas flow path 130 returning expired gases to the system, the system controller 1010 determines the first breathing circuit including the first patient interface 120 to be attached to the patient. The first patient interface 120 may include a sealing mask 124 or endotracheal tube 126 as depicted in FIGS. 25 and 26 and a CO₂ sensor (not shown) is provided in the gas flow path of the expiratory conduit returning expired gases to the system. The CO₂ sensor may be located in the main body of the system, e.g. at the expired gas inlet, or anywhere along the length of the expired gas conduit 130. A nasal cannula 224 depicted in FIGS. 25 and 26 has nasal prongs 226 which may direct high flow breathing gases into the nares of patient 300.

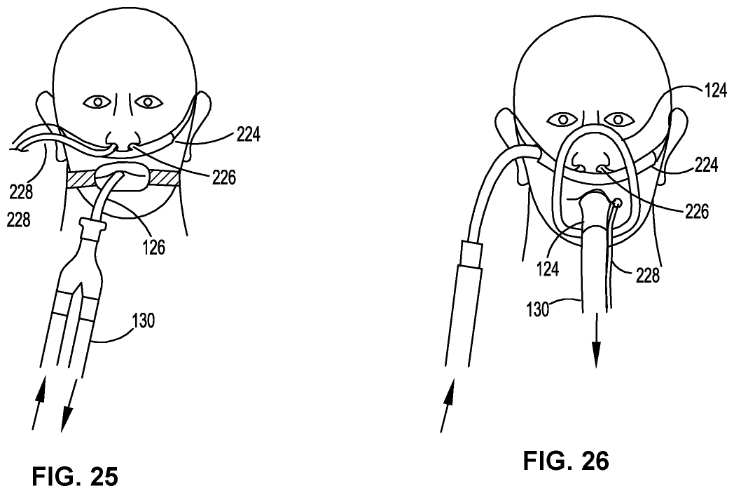

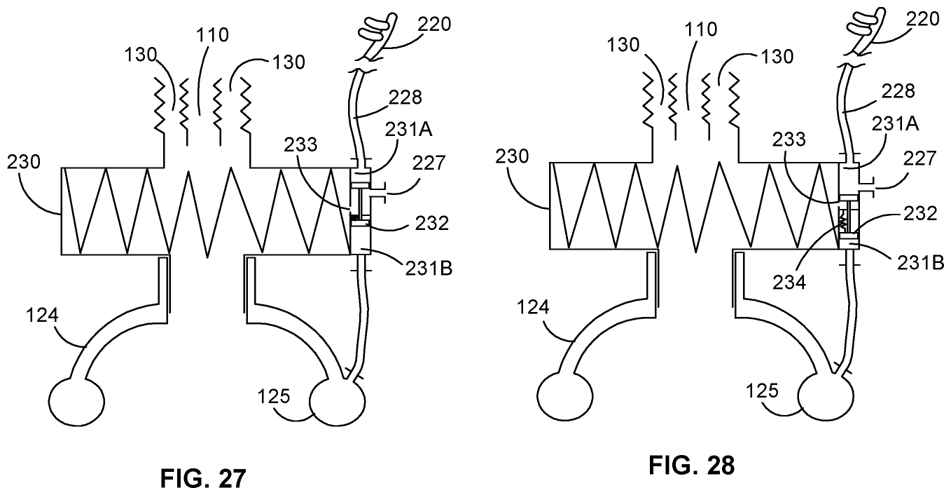

Since a non-sealing patient interface does not have return conduit for expired gases, a CO2 sensor may be located on the patient end of the interface, e.g. on a prong 226 or the cannula body to which the prongs attach. Alternatively, a sampling conduit 228 can be used to return sampled gas exiting the nares to a CO₂ sensor located elsewhere in the apparatus. The sampling conduit 228 may therefore connect with a gas sampling line 227 (FIGS. 27 and 28) which is in fluid communication with a CO₂ sensor providing input to system 1000 or it may provide fluid communication with the expiratory gas conduit 130 as depicted in FIG. 26. Since CO₂ will quickly dissipate into the atmosphere, in embodiments where the expired gas is directed to the expiratory gas conduit 130 via sampling conduit 228, the controller 1010 may be configured to associate a lower detected CO₂ concentration with use of the second, non-sealing patient interface 220/224.

The sampling conduit 228 may provide a flow into the expiratory gas conduit 130 or it may provide a separate conduit in fluid communication with a dedicated gas sampling inlet of system 1000. FIG. 26 shows cannula 224, and face mask 124 which is coupled with expiratory gas conduit 130 (for simplicity, the inspiratory gas conduit 110 is not shown). Sampling conduit 228 directs expired gases from the patient's mouth into the expiratory gas conduit 130 for sensing by a CO₂ sensor. In this arrangement, the nasal cannula 224 may be used to deliver a breathing gases which may include anaesthetic agents in a first mode with the mask providing means to direct expired gases via expiratory pathway 130. In some embodiments, sampling conduit 228 may be decoupled from a sealing mask 124 expiratory gas conduit and coupled with an endotracheal tube 126 expiratory gas conduit (and vice versa) which enable sensing during different phases of sedation including induction, ongoing anaesthesia with ventilation, and weaning.

A range of different approaches to sensing as a means to provide mode switching in a system for delivering breathing gases to a patient may be used. In one example, pressure in the mask may be monitored by a pressure sensor and used to detect when the mask is in place on the patient. The mask may be placed over the high flow cannula (as in FIG. 32) or it may replace the cannula. Detection of the mask on the patient may be achieved by detecting an increase in pressure from pressures expected during delivery of high flow respiratory support (consistent with a mask being placed over the cannula) and indicates a rebreathing circuit may be intended to be used and this may cause switching from high flow mode of operation of the respiratory support system 100 to a rebreathing mode. Additionally or alternatively, an optical sensor may be provided in the mask which is configured to monitor changes in emitted/detected light that occurs in the presence of the patient's skin, and may be used to detect placement of the mask on the patient. This may be used in conjunction with a pressure sensor in the cuff of a mask which detects an increase in pressure when the mask is applied to the patient which may trigger a switch in control from a high flow mode to a rebreathing mode. Likewise, if an opposite change in these measurements is detected then this may be consistent with the mask being removed from the patient which may trigger a switch in control from a rebreathing mode to a high flow mode.

Alternatively or additionally, sensing in the expiratory flow path of the rebreathing tube may be utilised to determine when the mask is in place on the patient to trigger the switch to rebreathing. The mask may be placed over the high flow cannula (as in FIG. 32) or it may replace the cannula. One or more parameters in the expiratory flow path such as flow rate, pressure, temperature, or humidity may be determined using suitable sensors. An increase in one or more of these parameters (e.g. if a sensor determines temperature in the expiratory flow path to have increased or to be higher than ambient), would indicate the mask is on the patient and trigger switching from high flow mode to rebreathing mode.

FIGS. 27 and 28 show another arrangement utilising a piston driven assembly to selectively direct expired gases from each of the first patient interface (mask 124) and second patient interface (nasal cannula 224) to a gas sampling line 227. A sampling conduit 228 collects expired gases from nasal cannula 224 into a first chamber 231A of the assembly. The assembly is housed with filter 230 (which may be omitted) that receives expired gases from face mask 124 and directs them in to first chamber 231A through an opening 233. A second chamber 231B is adjacent the first chamber and is in fluid communication with an air filled cuff 125 of mask 124. Piston 232 is movable between the two chambers 231A, 231B between a first position (FIG. 27) and a second position (FIG. 28). The first position occludes gas flow from sampling conduit 228 to gas sampling line 227 and expired gases from mask 124 are received through filter 230 and opening 233 into first chamber 231A and directed to gas sampling line 227 (FIG. 27). The second position occludes flow of expired gases from mask 124 to gas sampling line 227 and expired gases from sampling conduit 228 are directed to gas sampling line 227 (FIG. 28). When mask 124 is applied to the patient's face for delivery of breathing gases in the first mode, pressure in cuff 125 increases, shifting piston 232 away from the cuff to the first position. When the mask 124 is removed from the patient's face, biasing means 234 shifts plunger to the second position. It is to be understood that while biasing means 234 is shown as a resilient biasing means in the Figures, operation of the plunger may be electronically, mechanically or otherwise controlled.

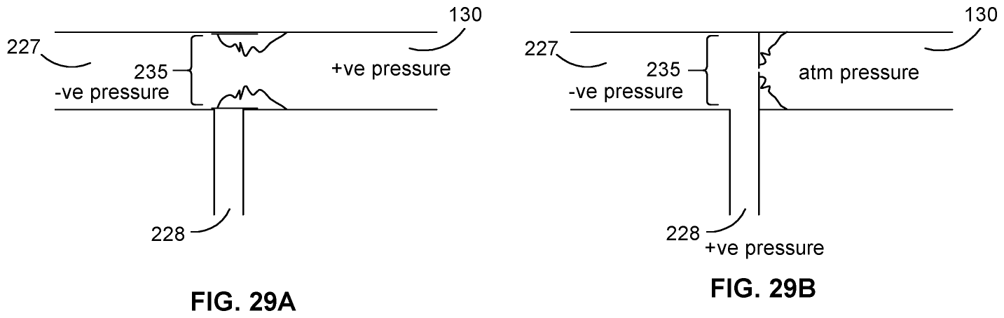

In another arrangement shown in FIGS. 29A and 29B, airway pressure as determined by pressure in the mask itself is used to selectively direct expiratory gas into gas sampling line 227. FIG. 29A shows the arrangement while mask is in use in the first mode, and FIG. 29B shows the arrangement while nasal cannula (not shown) is in use. Gas sampling line 227 has lower pressure than both the expiratory conduit 130 from the face mask and sampling conduit 228 from the nasal cannula. In FIG. 29A, positive pressure in the mask during use in the first mode causes a pressure responsive flow diverter 235 to open a flow of expiratory gas from expiratory conduit 130 into gas sampling line 227 while preventing a flow of expiratory gas from sampling conduit 228 (from the nasal cannula). In FIG. 29B, the mask is removed from the patient so that pressure in expiratory conduit 130 is equivalent to atmospheric pressure. During the second mode of operation, the nasal cannula is applied to the patient and expired gas in the sampling conduit 228 increases the pressure causing flow diverter 235 to open a flow of expiratory gas from sampling conduit 228 into gas sampling lie 227 while substantially preventing a flow of expiratory gas from expiratory conduit 130 due to the pressure differential and owing to the arrangement of flow diverter 235 when operated in the second mode.

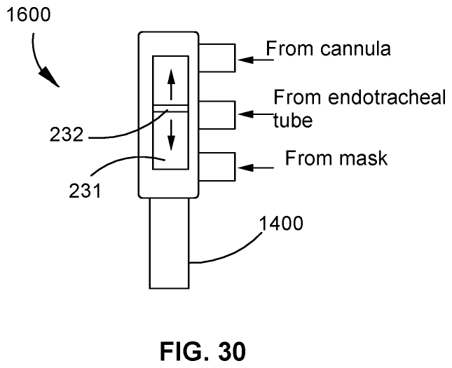

In yet another arrangement shown in FIG. 30, a three way switch 1600 is actuated by a user to selectively direct expiratory gas from one of a nasal cannula, an endotracheal tube, and a sealing mask into a gas sampling line 227 which, in a preferred embodiment, is incorporated into a multi-lumen assembly 1400. A piston 232 coupled with an actuator operated by the user moves within a chamber 231 inside the switch housing to direct expired gas flows according to the mode of operation or patient interface selected by the user. When the piston is moved most distally of the multi-lumen tube assembly 1400, high pressure expiratory gases from the cannula (via sampling conduit 228) enter the chamber 231 and gas sampling line 227 inside the multi-lumen assembly. When the piston is shifted toward the multi-lumen tube assembly 1400, flows from the cannula cannot enter the gas sampling line and instead, expiratory gases enter from one of the endotracheal tube or mask.

In some embodiments, the system 1000 includes a first respiratory apparatus 100 delivering breathing gases in the first mode, and a second respiratory apparatus 200 delivering breathing gases in the second mode as described herein. The first respiratory apparatus 100 may thus include one or more of a CO₂ absorber configured to treat returned expired gas before recirculating to the patient in the first mode, a pressure limiting valve configured to maintain substantially stable pressure in the system in the first mode; a variable volume (such as a ventilation bag or bellows) for displacement of gases in the first mode, a fresh gas flow for replenishing anaesthetic gas delivered to the patient in the first mode and a vaporizer for vaporizing volatile anaesthetic agents into breathing gas delivered to the patient in the first mode. The second respiratory apparatus 200 includes a flow modulator configured to provide high gas flows through the system and typically includes a humidifier configured to condition the breathing gas to a pre-determined temperature and/or humidity before delivery to the patient in the second mode.

In preferred embodiments, when the second mode is selected, the system isolates flow of breathing gases from the first respiratory apparatus to the patient so that there is no delivery of anaesthetic agents to the patient 300. In some embodiments the first respiratory apparatus 100 and the second respiratory apparatus 200 are integrated in a unitary machine although that need not be the case. In either case, a humidifier may be provided to condition the breathing gas to a pre-determined temperature and/or humidity before delivery to the patient in the second mode.

In some embodiments the system 1000 includes a display device or monitor 1094 in operative communication with the system controller 1010 and configurable to display one or more CO₂ traces based on inputs from the one or more CO₂ sensors received by the system controller. The system controller 1010 may automatically determine which of CO₂ sensor signals to display on the monitor 1094, selecting the trace corresponding to the CO₂ sensor input representing the highest detected CO₂ values from the plurality of CO₂ sensors or the CO₂ values most likely to resemble patient values (e.g. higher than ambient CO₂ concentration). Alternatively, the system controller 1010 may cause simultaneous or cycling display of all CO₂ traces on monitor 1094, or on one or more a separate monitors which may be devoted solely to CO₂ monitoring.

Respiratory Apparatus with Humidification

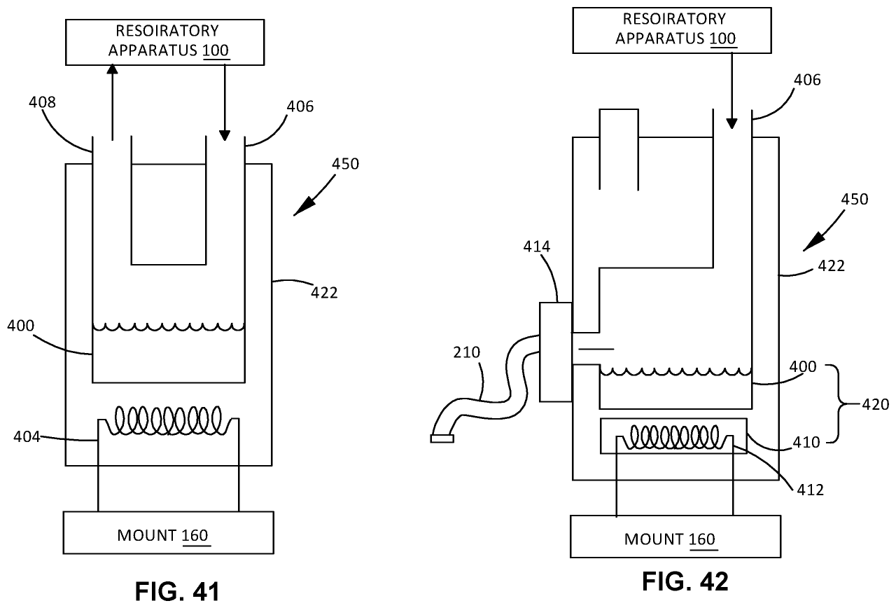

FIGS. 38 to 40 illustrate a respiratory apparatus 100 for delivering breathing gases to a patient 300 in accordance with an embodiment of the disclosure. The respiratory apparatus 100 includes a flow source 1030 for providing a flow of breathing gases in an inspiratory flow path for delivery to the patient 300. The respiratory apparatus 100 also includes a mount 160 for coupling with at least one vaporizer 150 for vaporizing one or more volatile anaesthetic agents into the flow of breathing gases in the inspiratory flow path before delivery to the patient 300. The respiratory apparatus 100 also includes a return path for recirculating expired gases received from the patient 300 via an expiratory flow path to the inspiratory flow path. The mount 160 is couplable with a humidification component 450 for conditioning the flow of breathing gases in the inspiratory flow path to a pre-determined temperature and/or humidity before delivery to the patient 300.

The components of the respiratory apparatus 100 as shown and described in relation to FIGS. 38 to 40 having similar numbering to the components of the anaesthesia machine 10, ventilator 20 and high flow system 30 are intended to refer to the same components. Thus, the description of those components in relation to the anaesthesia machine 10 is considered to be applicable to the respiratory apparatus 100 according to some embodiments of the disclosure.

The flow source 1030 of the respiratory apparatus 100 may include a flow modulator (such as flow modulator 250 described with reference to FIG. 2), or more particularly, a flow generator adapted to receive one or more breathing gases externally of the respiratory apparatus 100 and generate a gas flow through the respiratory apparatus 100. The flow generator may be in fluid communication directly or indirectly with a gas supply 1060 for providing the one or more breathing gases to the respiratory apparatus 100. The gas supply 1060 may be a source of gases supplied by one or more hospital gas outlets, such as located in an operating theatre or ICU. The gas supply 1060 may be configured to supply nitric oxide (NO), oxygen (O2) and/or air to the flow generator.

In some embodiments, the flow source 1030 includes the gas supply 1060. The flow source 1030 and/or respiratory apparatus 100 may include one or more valve arrangements adapted to control the rate at which one or more gases, e.g., one or more of NO, O2 and air supplied from the gas supply 1060, are provided to the inspiratory flow path. This may also allow for control of mixing of the one or more gases to a desired composition for use by the respiratory apparatus 100. In alternative embodiments, the flow source 1030 may not include the gas supply 1060. Instead, the flow source 1030 may include one or more containers of compressed air and/or another gas and one or more valve arrangements adapted to control the rate at which the gases leave the one or more containers to provide the flow of breathing gases to the inspiratory flow path.

In other embodiments, the respiratory apparatus 100 is in gas flow communication with a gas delivery apparatus 1040 that is in fluid communication with the gas supply 1060, as illustrated by the broken lines shown in FIG. 38. The gas delivery apparatus 1040 may receive a supply of gas including one or more of NO, O2 and air from the gas supply 1060. The gas delivery apparatus 1040 includes a gas mixing element 1042 for combining one or more of NO, O2 and air from the gas supply 1060 in a proportion required for delivering the breathing gases in a desired proportion for use by the respiratory apparatus 100. For example, when the respiratory apparatus 100 is operating to provide anaesthetic ventilation with one or more anaesthetic agents to the patient 300, the gas mixing element 1042 may include at least nitric oxide (NO) in the breathing gases provided to the respiratory apparatus 100. Alternatively, when the respiratory apparatus 100 is operating to provide anaesthetic ventilation without anaesthetic agents to the patient 300, the gas mixing element 1042 may include O2 and/or air.

The gas delivery apparatus 1040 also includes one or more flow meters 1090 for controlling the flow of the breathing gases provided to the respiratory apparatus 100 as shown in FIG. 38. The gas delivery apparatus 1040 may include a flow meter 1090 for controlling the gas lines delivering each of NO, O2 and air (see e.g., FIGS. 54 and 55 with flow meters 190 of the respiratory apparatus 100). The flow meters 1090 may control the gas mixing by changing the flow rate of each of the breathing gases provided to the respiratory apparatus 100, and thereby, the proportion of the breathing gases. The gas delivery apparatus 1040 may also include a gas outlet 1044 couplable with the respiratory apparatus 100 for supplying gas from the gas delivery apparatus 1040 to the respiratory apparatus 100.

In other embodiments, the respiratory apparatus 100 may include the gas delivery apparatus 1040. The gas delivery apparatus 1040 may be in fluid communication with the flow source 1030 for providing the breathing gases to the flow source 1030 for generating a gas flow in the inspiratory flow path for delivery to the patient 300. Alternatively, the gas delivery apparatus 1040 may replace the flow source 1030 such that the gas outlet 1044 directly provides the flow of breathing gases to the inspiratory flow path.

The respiratory apparatus 100 provides an inspiratory flow path through which a flow of breathing gases is directed into the patient's airway 310. The flow of breathing gases from the flow source 1030 may be conditioned and/or modified in the inspiratory flow path prior to delivery to the patient 300. As illustrated in FIG. 38, the flow of breathing gases may be directed from the flow source 1030 (or gas supply 1060 and/or gas delivery apparatus 1040) to a vaporizer 150, a humidification component 450 external to the respiratory apparatus 100, or directly to an inspiratory conduit 110 or an inspiratory conduit 120. The options for directing the flow of breathing gases may be controlled by the controller 1010 of the respiratory apparatus 100. Furthermore, a switching mechanism 1020 may be provided to control switching between the options for directing the flow of breathing gases in the inspiratory flow path. This will be described in more detail in relation to FIGS. 52 and 49.

The respiratory apparatus 100 is operable in a number of modes of operation, e.g., by the controller 1010. The respiratory apparatus 100 may be operable in a first mode in which the respiratory apparatus 100 delivers breathing gases to the inspiratory flow path and receives return of expired gases via the expiratory flow path. The first mode of operation may be an anaesthetic ventilation mode, which may include providing ventilatory support with or without one or more anaesthetic agents. Typically, the breathing gases are delivered at a low flow rate, e.g., of less than about 15 L/min. In the first mode, the breathing gases may include one or more of NO, O2 and air. The breathing gases may optionally include one or more anaesthetic agents, such as nitric oxide from the flow source 1030 and/or one or more volatile agents from the vaporizer 150, for operation of the respiratory apparatus 100 in the anaesthetic ventilation mode.

In the first mode, the flow of breathing gases may be directed from the flow source 1030 to either the vaporizer 150 or directly to the inspiratory conduit 110 for delivery to the patient 300 as shown in FIG. 38. When the flow of breathing gases is directed to the at least one vaporizer 150, the flow of breathing gases may be modified to optionally include one or more volatile anaesthetic agents which are vaporized into the gas flow. The one or more volatile anaesthetic agents may include isoflurane or sevoflurane which is converted from liquid to vapour by the vaporizer 150. The flow of breathing gases with the optional anaesthetic agents is then directed to the inspiratory conduit 110 for delivery to the patient's airway 310 via a first patient interface 120. The first patient interface 120 may form a sealing interface with the patient's airway 310, and may include a mask or endotracheal tube. The first patient interface 120 may include a laryngeal mask airway (LMA) or a sealing face mask.

The respiratory apparatus 100 also provides an expiratory flow path through which a flow of expired gases is received from the patient 300. The first patient interface 120 may be configured to receive expired gases from the patient 300 in the first mode as illustrated in FIG. 38. The expired gases may flow through an expiratory conduit 130 which is coupled with rebreathing components 140 of the respiratory apparatus 100. The rebreathing components 140 includes at least a CO2 absorber 141 configured to treat returned expired gas from the patient 300 before recirculating to the inspiratory flow path in the first mode. The rebreathing components 140 may also include one or more of a ventilation bag 142, a pressure relief valve 143, a scavenger system 144, a bellows 145 and a pressure relief valve 146 as described in relation to the anaesthesia machine 10 of FIG. 1A. Thus, the respiratory apparatus 100 provides a return path for recirculating expired gases from the patient 300 via the rebreathing components 140 to the inspiratory flow path in the first mode.

The respiratory apparatus 100 may also be operable, e.g., by the controller 1010, in a second mode in which the respiratory apparatus delivers breathing gases to the inspiratory flow path at a pre-determined flow rate without return of expired gases from the patient 300. The second mode may be a high flow mode for delivery of high flow respiratory support. Typically, the breathing gases are delivered at a high flow rate, such as in the range of about 20 L/min to about 90 L/min, or in the range of about 40 L/min to about 70 L/min. In other embodiments, the pre-determined flow rate may include about 20 L/min, about 40 L/min or about 70 L/min. The breathing gases may include O2 and/or air in a proportion required for operation of the respiratory apparatus 100 in the second mode.

In the second mode, the flow of breathing gases may be directed from the flow source 1030 to the humidification component 450, and the breathing gases are then directly or indirectly provided to the second inspiratory conduit 210 for delivery to the patient 300 as shown in FIG. 38. When the flow of breathing gases is directed to the humidification component 450, the humidification component 450 is operable to condition the flow of breathing gases in the inspiratory flow path to a pre-determined temperature and/or humidity before delivery to the patient 300. The humidification component 450 may be operable to heat the flow of breathing gases to the pre-determined temperature. The pre-determined temperature and/or humidity may include temperature and/or humidity values or ranges of values that are suitable for delivery of ventilatory support, and particularly high flow respiratory support, to the patient 300. However, it is to be understood that in some embodiments it may be desirable to enable the humidification component 450 to provide humidification and/or warming of gas during delivery of support in the first, rebreathing mode, as well as enabling the vaporizer 150.

The humidification component 450 may operate in at least an invasive mode (for example, for patients with a bypassed airway) and/or a non-invasive mode (for example, for patients or users with breathing masks or nasal cannulas). Each mode can have a number of humidity settings, which can be expressed as a dew point or absolute humidity. The humidification component 450 may be controlled to deliver, at an outlet port 408 of the humidification chamber 400 and/or the patient end of the inspiratory conduit 210, humidified gases having a dew point (or absolute humidity) at or near a predetermined humidity level. For example, a user or clinician can select a setting appropriate for the current mode of operation. A number of humidity settings may be provided. For example, the humidity settings may be equivalent to a dew point of 37° C., 31° C., 29° C., 27° C., or others. The humidity setting equivalent to a dew point of 37° C. may be suitable for invasive therapy (that is, where the patient's upper airways are bypassed) whereas the other humidity settings may be suitable for non-invasive respiratory support, although the humidity settings may not be restricted to a particular type of respiratory support. Alternatively, each humidity setting may be continuously variable between upper and lower limits. A lower humidity setting may be selected by the user or clinician to reduce condensation or “rain-out” in the inspiratory conduit 210, or a higher humidity setting may be selected to improve patient comfort or physiological benefits. Some humidification components 450 disclosed herein can also include a high flow, unsealed mode, or any other modes known to those persons skilled in the art.

The humidification component 450 may provide different levels of humidity for different respiratory support applications. For example, the humidification component 450 can deliver a desired humidity level of about 44 mg/L BTPS (about 37° C. fully saturated) for invasive and/or high flow respiratory support and/or about 32 mg/L BTPS (about 31° C. fully saturated) for non-invasive forms of respiratory support. Other suitable patient comfort settings could also be delivered for various forms of respiratory support.

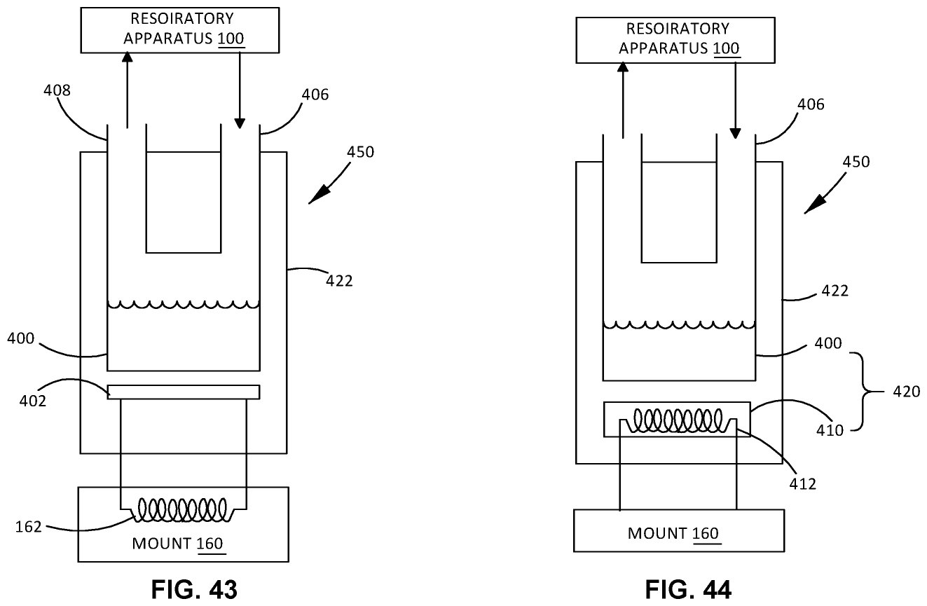

The conditioned flow of breathing gases from the humidification component 450 may be directed to the second inspiratory conduit 210 and a second patient interface 220 couplable with the patient's airway 310. The second patient interface 220 may form a non-sealing interface with the patient's airway 310, and may include a nasal cannula. Alternatively, the conditioned flow of breathing gases may be returned to the respiratory apparatus 100 and then exit through a dedicated outlet 164 for delivery to the patient 300 (see also FIG. 39). In this case, the second inspiratory conduit 210 may be coupled with the outlet 164 to deliver the conditioned flow of breathing gases to the patient's airway 310. Additionally/alternatively, the respiratory apparatus 100 may also enable the conditioned flow of breathing gases from the humidification component 450 to be directed to the first inspiratory conduit 110 in the first mode of operation. Thus, some humidification function may also be utilised in the first mode of operation if desired by the anaesthetist or clinician.

Importantly, the respiratory apparatus 100 does not allow the flow of breathing gases with anaesthetic agents to be directed to the patient 300 in the second mode in some embodiments. Operation of the humidification component 450 may prevent delivery of the one or more volatile anaesthetic agents into the flow of breathing gases in the inspiratory flow path. More particularly, operation of the humidification component 450 may disable operation of the at least one vaporizer 150, or all of the vaporizers 150 in the respiratory apparatus 100. For example, the respiratory apparatus 100 may include a one-way valve or other arrangement to prevent passage of gas flow, especially any volatile anaesthetic agents, from the vaporizer 150 to the second inspiratory conduit 210 when the humidification component 450 is in operation. Such arrangements have been described in the foregoing.

The respiratory apparatus 100 may advantageously enable selective operation of the one or more vaporizers 150 and the humidification component 450. The respiratory apparatus 100 may include an optional switching mechanism 1020 as illustrated in FIG. 38 to enable selective operation of the vaporizer 150 or the humidification component 450, and if neither are selected, the gas flow is directed to the inspiratory conduit 110 or the inspiratory conduit 210 as shown. The switching mechanism 1020 may be configured for operation depending on the mode of operation of the respiratory apparatus 100, which will be described in more detail.

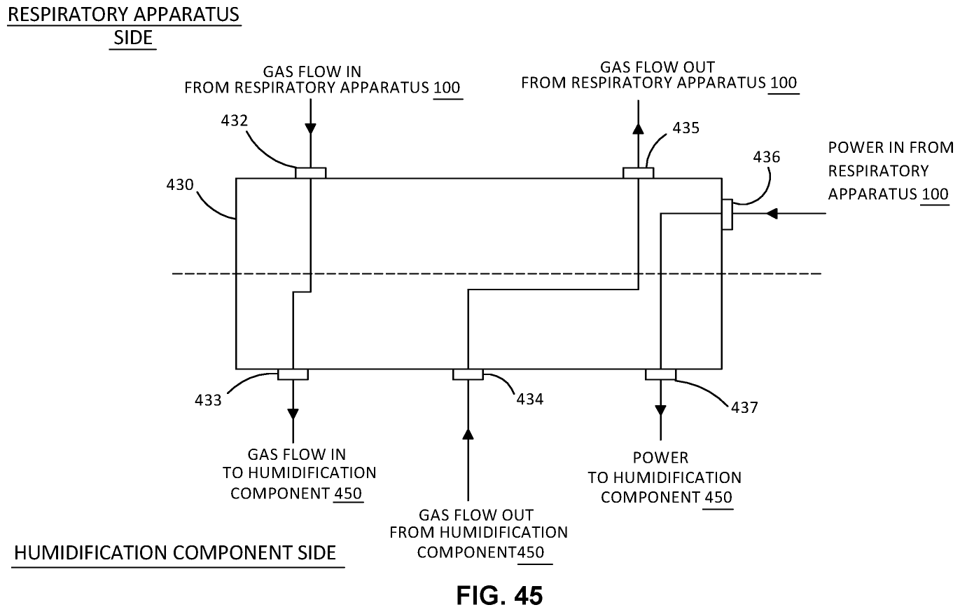

FIGS. 39 and 40 illustrate additional components of the respiratory apparatus 100, depicted as an anaesthesia machine, with similar features to the anaesthesia machine 10 shown in FIG. 1A. The respiratory apparatus 100 may include a module 180 having an auxiliary O2 flow meter and suction regulator. The respiratory apparatus 100 may also be configurable in gas flow communication with one or more flow meter(s) 190 (see also FIGS. 54 and 55) similar to the operation of the flow meter(s) 1090 of the gas delivery apparatus 1040 as shown and described in relation to FIG. 38 for controlling the flow rate/mixing of gases in the respiratory apparatus 100. The respiratory apparatus 100 may be configurable in gas flow communication with a flow meter 190 for controlling the flow of gas including one or both of air and O2 in the second mode.

The respiratory apparatus 100 may be configurable in gas flow communication with one or more of a pressure limiting valve 143, 146 configured to maintain substantially stable pressure in the respiratory apparatus 100 in the first mode, a variable volume or bellows 145 for displacement of gases in the first mode, a fresh gas flow (FGF) for replenishing breathing gases delivered to the patient 300 in the first mode (see also FIGS. 56 and 57), and a vaporizer 150 for vaporizing one or more volatile anaesthetic agents into the flow of the breathing gases before delivery to the patient 300 in the first mode. For example, see also componentry of the anaesthesia machine 10 of FIG. 1A. The respiratory apparatus 100 may also include two monitors, namely a patient monitor 192 and a system monitor 194, for displaying information useful to an operator of the respiratory apparatus 100.

The respiratory apparatus 100 also includes a mount 160 which is couplable with a vaporizer 150 and a humidification component 450 as shown in FIG. 39. The mount 160 may include a plurality of slots for receiving a housing of the vaporizer 150 and the humidification component 450. The housings of the vaporizer 150 and humidification component 450 may be slidably received in the slots of the mount 160. An empty slot of the mount 160 is illustrated in FIG. 40 where the humidification component 450 has been removed. The mount 160 may enable at least one vaporizer 150 and the humidification component 450 to be mountable adjacent to one another on the respiratory apparatus 100 as shown in FIG. 39. The vaporizer 150 and the humidification component 450 may be positioned in a side-by-side arrangement on the mount 160. The housings of the vaporizer 150 and the humidification component 450 or components thereof may cooperatively engage with one another, as will be described in relation to FIGS. 46 to 51.

In some embodiments, the mount 160 is couplable with two or more vaporizers 150 (not shown), in addition to the humidification component 450. The humidification component 450 may be positioned on the mount 160 with a vaporizer 150 located on either side of the humidification component 450. This may enable the housing of the humidification component 450 to cooperatively engage with each of the housings of the vaporizers 150, such as to disable operation of both vaporizers 150 when the humidification component 450 is in operation. This will be described in more detail in relation to FIGS. 46 to 51. The mount 160 may also include a heating element 162 shown as a heating coil in FIG. 40, which will be described in more detail in relation to FIG. 43. However, the heating element 162 is optional and may not be included in the mount 160.

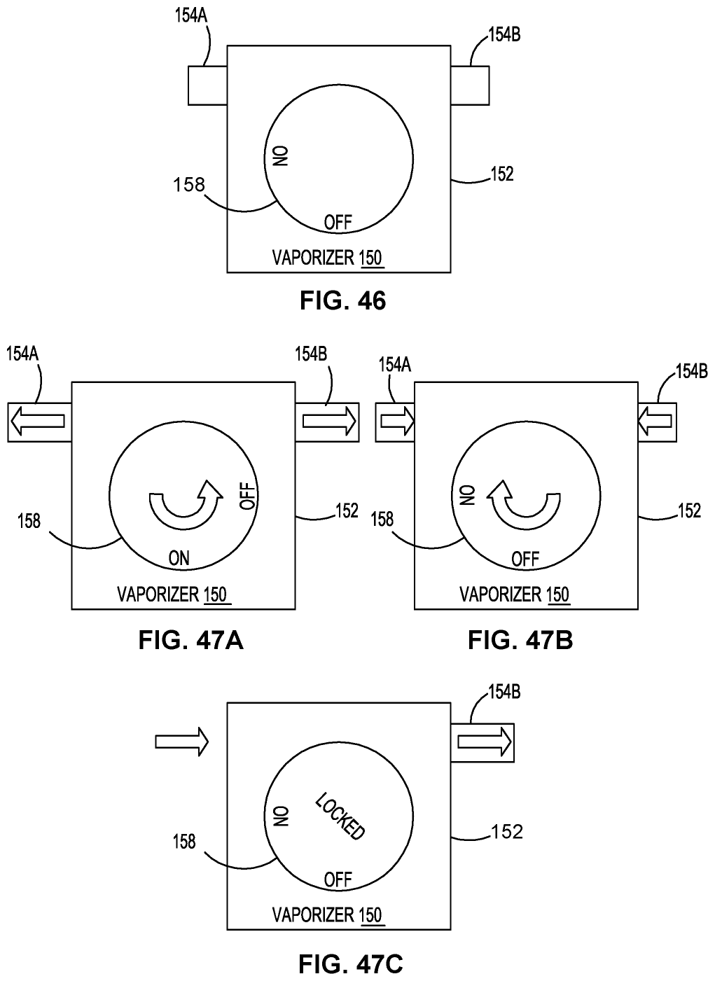

FIGS. 41 to 44 illustrate different embodiments of a humidification component 450 for use with a respiratory apparatus 100 for delivering breathing gases to a patient 300, according to some preferred embodiments of the disclosure. The respiratory apparatus 100 includes a flow source 1030 for providing a flow of breathing gases in an inspiratory flow path for delivery to the patient 300. The respiratory apparatus 100 also includes a mount 160 for coupling with at least one vaporizer 150 for vaporizing one or more volatile anaesthetic agents into the flow of breathing gases in the inspiratory flow path before delivery to the patient 300. The respiratory apparatus 100 also includes a return path for recirculating expired gases received from the patient 300 via an expiratory low path to the inspiratory flow path. The humidification component 450 is couplable with the mount 160 of the respiratory apparatus 100 for conditioning the flow of breathing gases in the inspiratory flow path to a pre-determined temperature and/or humidity before delivery to the patient 300.

The humidification component 450 may be configured for use with the respiratory apparatus 100 of FIGS. 38 to 40 and as described in the embodiments herein. Accordingly, the description of the components of the respiratory apparatus 100 will not be repeated again for conciseness. The humidification component 450 may include a humidification chamber 400 as shown in FIGS. 39 and 41 to 44, through which breathing gases are received and conditioned to the pre-determined temperature and/or humidity. For example, in the embodiment of FIG. 39, the humidification chamber 400 may be built into the housing of the humidification component 450 and/or be a replaceable component. A user may be able to refill the liquid in the humidification chamber 400. In some embodiments, the humidification chamber 400 is configured to be coupled with the mount 160, and may be slidably received onto the mount 160, such as via the housing 422 being received in a slot of the mount 160. The humidification chamber 400 with housing 422 may be slidably received in the slot along a substantially horizontal plane with the mount 160.

FIG. 41 illustrates an embodiment of the humidification component 450 which includes a housing 422 with a humidification chamber 400 through which breathing gases are received from the respiratory apparatus 100 and conditioned to the pre-determined temperature and/or humidity. The humidification chamber 400 includes an inlet port 406 for receiving a flow of breathing gases from the respiratory apparatus 100, and a return port 408 couplable with the respiratory apparatus 100 for returning the conditioned flow of breathing gases. The humidification chamber 400 includes a heating element 404, such as a heating coil, to heat liquid in the humidification chamber 400. The humidification chamber 400 is configured to electrically connect with the respiratory apparatus 100 for operation of the humidification chamber. The heating element 404 is electrically connected to the mount 160 as shown in FIG. 41.

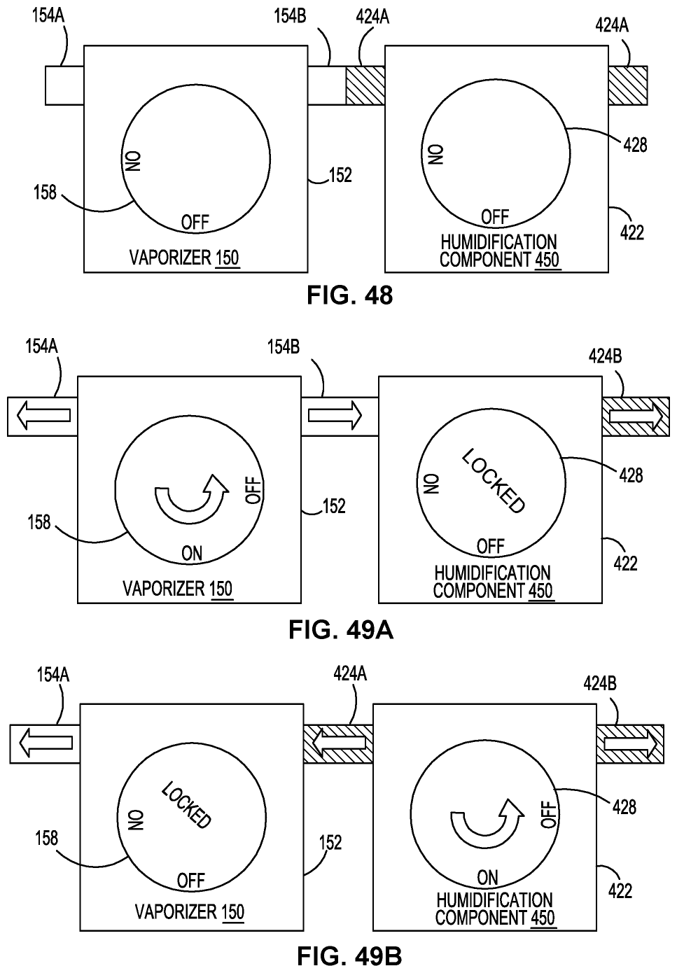

FIG. 42 illustrates another embodiment of the humidification component 450 which includes a housing 422 including a humidifier 420 having a humidification chamber 400 couplable with a humidification base unit 410 for operation of the humidification chamber 400. The humidification chamber 400 may be slidably couplable with the humidification base unit 410. The humidification chamber 400 may be slidably couplable such that it is received along a substantially horizontal plane of the humidification base unit 410. For example, U.S. Pat. No. 5,445,143 disclosed a humidification chamber operated on a base in a substantially horizontal plane which would be suitable for embodiments of the present disclosure, the disclosure of which is incorporated herein by this reference.

In some embodiments, the humidification base unit 410 is configured to be coupled with the mount 160. A housing of the humidification base unit 410 may be configured to be slidably received onto the mount 160. For example, the mount 160 may include a plurality of slots and the housing of the humidification base unit 410 may be slidably receive into one of the slots, such as shown by the mount 160 of FIG. 40. The humidification base unit 410 or a housing thereof may be received in a similar slidable manner as the humidification component 450 described in relation to FIGS. 39 and 40.

The humidification chamber 400 includes an inlet port 406 for receiving a flow of breathing gases from the respiratory apparatus 100, and an outlet port 414 for delivering the conditioned flow of breathing gases to the patient 300. The outlet port 414 is couplable with the inspiratory conduit 210 for delivering the conditioned flow of breathing gases to the patient 300 via a patient interface (e.g., a sealing or non-sealing patient interface). The inspiratory conduit 210 may be coupled with a second patient interface 220, for example, a nasal cannula, for delivering breathing gases to the patient's airway 310. The humidification base unit 410 includes a heating element 412, such as a heating coil, to heat liquid in the humidification chamber 400. The heating element 412 of the humidification base unit 410 is electrically connected to the mount 160 as shown in FIG. 42.

FIG. 43 illustrates another embodiment of the humidification component 450 which includes a housing 422 having a humidification chamber 400 including an inlet port 406 and return port 408, similar to FIG. 41. In this embodiment, the humidification chamber 400 includes a conductive plate 402 which is coupled with a heating element 404 in the mount 160, such as shown in FIG. 40. The conductive plate 402 becomes heated upon operation of the heating element 404 in the mount 160 and transfers the heat to the humidification chamber 400 to heat liquid in the humidification chamber 400.

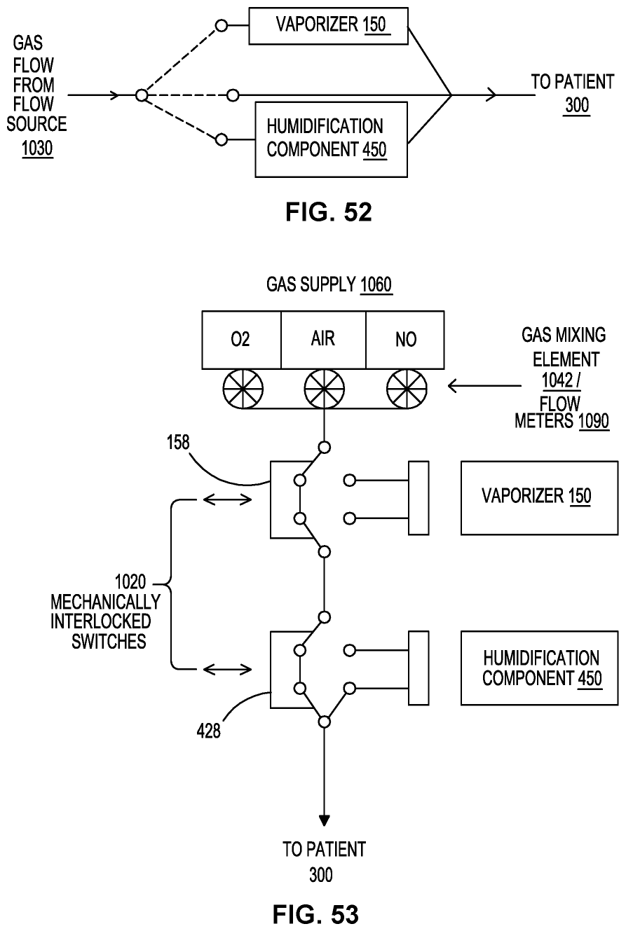

FIG. 44 illustrates another embodiment of the humidification component 450 similar to FIG. 42. In this embodiment, the humidification chamber 400 includes a return port 408 for returning the conditioned flow of breathing gases to the respiratory apparatus 100. The outlet port 414 is excluded and unable to be coupled with the patient's airway 310. In this embodiment, the respiratory apparatus 100 may include an outlet port 164 as shown in FIG. 39 through which the conditioned flow of breathing gases from the humidification component 450 is delivered to the inspiratory flow path. The outlet port 164 may be coupled with the inspiratory conduit 210 and deliver the flow of breathing gases to the patient's airway 310 via the second patient interface 220 as shown in FIG. 38.

In some embodiments, the humidification chamber 400 includes a liquid inlet that connects to a liquid reservoir for refilling of the humidification chamber 400. The humidification chamber 400 may also include a flow control mechanism to control flow of liquid into the humidification chamber 400. The humidification chamber 400 may include at least one sensor for detecting a level of liquid in the humidification chamber 400. The humidification chamber 400 may include a float valve for controlling a level of liquid in the humidification chamber 400. For example, U.S. Pat. No. 5,445,143 disclosed a dual float valve humidification chamber which would be suitable for embodiments of the present disclosure, the disclosure of which is incorporated herein by this reference.

In some embodiments, the humidification component 450 is couplable with the mount 160 via an adapter 430. FIG. 45 illustrates an exemplary adapter 430 for coupling a humidification component 450 with the respiratory apparatus 100 for delivering breathing gases to a patient 300. The adapter 430 may be couplable with the mount 160 of the respiratory apparatus 100. The adapter 430 may be received in a slot of the mount 160 in place of a vaporizer 150 or the humidification component 450.

As illustrated in FIG. 45, the adapter 430 includes a first inlet port 432 for receiving a flow of the breathing gases from the respiratory apparatus 100, and a first outlet port 433 for delivering the flow of breathing gases to the humidification component 450. The adapter 430 further includes a second inlet port 434 for receiving a conditioned flow of breathing gases from the humidification component 450. The conditioned flow of breathing gases is delivered to the patient 300 or to the respiratory apparatus 100 via a second outlet port 435 on the adapter 430. In some embodiments, the second inlet port 434 and the second outlet port 435 are not required when the humidification component 450 directly couples with the inspiratory conduit 210 to deliver the conditioned flow of breathing gases to the patient's airway 310 (see, e.g., FIG. 42).

FIG. 45 illustrates that the adapter 430 is configured to electrically connect with the respiratory apparatus 100 for operation of the humidification component 450. For example, the adapter 430 as shown in FIG. 45 includes a first power connector 436 for electrically connecting the adapter 430 with the respiratory apparatus 100. The adapter 430 also includes a second power connector 437 for electrically connecting the humidification component 450 with the adapter 430. Accordingly, the humidification component 450 may be powered through the adapter 430 connected with the respiratory apparatus 100. It is to be understood, however, that the humidification may alternatively/additionally be configured to electrically connect with a battery or other self-contained power supply as would be understood by one of skill in the art.

Advantageously, in some embodiments of the disclosure, operation of the humidification component 450 prevents delivery of the one or more volatile anaesthetic agents into the flow of breathing gases in the inspiratory flow path. More particularly, operation of the humidification component 450 may disable operation of the at least one vaporizer 150, as described in relation to FIG. 38. The respiratory apparatus 100 may include an interlocking mechanism to prevent simultaneous operation of the humidification component 450 and the at least one vaporizer 150. The interlocking mechanism may be configured to enable operation of the humidification component 450 or the at least one vaporizer 150 when in an unlocked configuration, and to disable operation of the humidification component 450 or the at least one vaporizer 150 when in a locked configuration. FIGS. 46 to 49 illustrate an exemplary interlocking mechanism of the respiratory apparatus 100 according to some embodiments of the disclosure.

FIG. 46 illustrates a vaporizer 150 including a housing 152 having a dial 158 to provide an ON/OFF switch when rotated by a user. The vaporizer 150 includes a locking element associated with the housing 152. The locking element 152 includes two locking pins 154A and 154B that are retractable within the housing 152 in the locked configuration and extendable from the housing 152 in the unlocked configuration. Each locking pin 154A and 154B may be independently retractable within the housing 152 in the locked configuration and extendable from the housing 152 in the unlocked configuration. In some embodiments, the locking element 152 may include only a single locking pin, such as locking pin 154B, as would be appreciated by a person skilled in the art.

FIGS. 47A-C are schematic diagrams illustrating operation of the interlocking mechanism of the vaporizer 150 of FIG. 46. In FIG. 47A, the dial 158 is rotated to the ON position and the locking pins 154A and 154B extend from the housing 152. In FIG. 47B, the dial 158 is rotated to the OFF position and the locking pins 154A and 154B retract towards the housing 152. In FIGS. 47A and 47B the vaporizer 150 remains in an unlocked configuration in which the dial 158 is able to be rotated between the ON/OFF positions by a user to enable or disable operation of the vaporizer 150. In FIG. 47C, an external force is applied to the locking pin 154A such that the pin becomes fully retracted within the housing 152 and is not visible. The external force may be applied by a user, or more preferably, is applied by a corresponding locking element associated with a housing 422 of the humidification component 450, as will be described. In this state, the vaporizer 150 is now in a locked configuration in which the dial 158 is disabled and unable to be rotated between the ON/OFF positions. The vaporizer 150 is in a locked configuration in the OFF position on the dial 158. The vaporizer 150 will only be able to be operated when the external force is removed and/or the locking pin 154A is released from within the housing 152.

FIG. 48 illustrates the vaporizer 150 of FIG. 46 positioned adjacent to a humidification component 450. The humidification component 450 includes a housing 422 having a dial 428 to provide an ON/OFF switch when rotated by a user. The humidification component 450 includes a locking element associated with the housing 422. The locking element includes two locking pins 424A and 424B that are retractable within the housing 422 in the locked configuration and extendable from the housing 422 in the unlocked configuration. In some embodiments, the locking element includes only a single locking pin, such as locking pin 424A, as would be appreciated by a person skilled in the art.

The vaporizer 150 and the humidification component 450 may include the same locking element as shown in FIG. 48. The vaporizer 150 and the humidification component 450 may be mountable adjacent to one another on the respiratory apparatus 100, such as by coupling adjacent to one another on the mount 160 as shown in FIG. 39, to enable cooperation with one another. Accordingly, the vaporizer 150 and the humidification component 450 may be configured for cooperation with one another to provide the interlocking mechanism. Moreover, the locking element of the vaporizer 150 or the humidification component 450 may be configured to engage with a corresponding locking element of the other of the vaporizer 150 or the humidification component 450 to provide the interlocking mechanism.

FIGS. 49A-B are schematic diagrams showing the vaporizer 150 and humidification component 450 with the interlocking mechanism of FIG. 48. In FIG. 49A, the vaporizer 150 is switched to the ON position by rotating the dial 158 and the locking pins 154A and 154B extend from the housing 152. Since the vaporizer 150 is located adjacent to the humidification component 450, the locking pin 154B of the vaporizer 150 presses against the locking pin 424A of the humidification component 450 and causes it to retract within the housing 422 of the humidification component 450 and is not visible in FIG. 49A. In this state, the humidification component 450 is now in a locked configuration in which the dial 428 is disabled and unable to be rotated between the ON/OFF positions. The humidification component 450 is in a locked configuration in the OFF position. The humidification component 450 will only be able to be operated when the vaporizer 150 is switched off by rotating the dial 158 to the OFF position to enable the locking pin 424A to extend from the housing 422.

FIG. 49B illustrates the opposite configuration of the interlocking mechanism of FIG. 49A. In this embodiment, the humidification component 450 is switched to the ON position by rotating the dial 428 and the locking pins 424A and 424B extend from the housing 422. The locking pin 424A presses against the locking pin 154B of the vaporizer 150 and causes it to retract with the housing 152 of the vaporizer 150 and is not visible in FIG. 49B. In this state, the vaporizer 150 is now in a locked configuration in which the dial 158 is disabled and unable to be rotated between the ON/OFF positions. The vaporizer 150 is in a locked configuration in the OFF position. The vaporizer 150 will only be able to be operated when the humidification component 450 is switched off by rotating the dial 428 to the OFF position to enable the locking pin 154B to extend from the housing 422.

It is advantageous for the locking element of the vaporizer 150 and the humidification component 450 to include a pair of locking pins, with a pin on each side of the housing of the vaporizer 150 and humidification component 450. This is useful as the mount 160 may concurrently receive one or more vaporizers 150 as well as the humidification component 450. If the humification component 450 is positioned between the two vaporizers 150, this enables the vaporizers 150 to both be disabled upon operation of the humidification component 450, as the locking pins 424A and 424B will cause retraction one of the locking pins 154A and 154B of the two vaporizers 150.

It is to be understood that the humidification component 450 and the vaporizer 150 may be operable using manual or electronic switching control means to achieve various operational states, such as wherein operation of the humidification component 450 is enabled while the vaporizer 150 is enabled, or wherein operation of the humidification component 450 is disabled while the vaporizer 150 is enabled, or wherein operation of the humidification component 450 is enabled while the vaporizer 150 is disabled, or operation of the humidification component 450 is disabled while the vaporizer 150 is disabled.

FIGS. 50 and 51 illustrate another interlocking mechanism according to some embodiments of the disclosure. In FIGS. 50 and 51, the vaporizer 150 and the humidification component 450 each include a slot associated with their respective housings. FIGS. 50 and 51 only illustrate a dial 158 of the vaporizer 150 and a dial 428 of the humidification component 450 for simplicity, excluding the respective housings. The dials 158, 428 are able to be rotated by a user to switch the vaporizer 150 and humidification component 450 to an ON/OFF position. The dial 158 of the vaporizer 150 includes a slot 156 and the dial 428 of the humidification component 450 includes a slot 426. A locking pin 460 is slidably movable between the slots 156, 426 to provide the interlocking mechanism.

As illustrated in FIG. 51A, the locking pin 460 is positionable within the slot 426 of the humidification component 450 by sliding the pin 460 from the slot 156 to the slot 426. A user may be able to operate the locking pin 460 and slide it between the slots 156, 426. In this state, the humidification component 450 is in the locked configuration as the dial 428 is unable to be rotated due to the presence of the locking pin 460. The vaporizer 150 is in the unlocked configuration as the dial 158 can be rotated between an ON/OFF position. In FIG. 51B, the locking pin 460 is positionable within the slot 156 of the vaporizer 150. In this state, the vaporizer 150 is in the locked configuration as the dial 158 is unable to be rotated due to the presence of the locking pin 460. In contrast, the humidification component 450 is in the unlocked configuration and the dial 428 is able to be rotated as shown to an ON position, thereby preventing operation of the vaporizer 150.

In other embodiments, the interlocking mechanism and the locking elements as illustrated in FIGS. 46 to 51 may incorporate different mechanism for operation as would be appreciated by a person skilled in the art. For example, the locking elements may include one or more resilient members or springs instead of locking pins. The locking elements may include other forms of mechanical interlocking components such as levers, bars, latches and locks to name a few.

FIG. 52 illustrates that the respiratory apparatus 100 may include a switching mechanism 1020 (not shown, see also FIGS. 38 and 53) configured to enable selective operation of the humidification component 450 and the at least one vaporizer 150. In some embodiments, the respiratory apparatus 100 may include two or more vaporizers 150 and the switching mechanism 1020 may selectively operate the two or more vaporizers 150, as well as the humidification component 450. The switching mechanism 1020 may direct the flow of breathing gases from the flow source 1030 (or from the gas delivery apparatus 1040 as previously described) to one of the vaporizers 150, the humidification component 450 or directly to the patient 300 via the first inspiratory conduit 110 or the second inspiratory conduit 120, depending on the mode of operation (see also FIG. 38).

Upon activation of the switching mechanism to operate the humidification component 450, the vaporizer(s) 150 may be prevented from operating until the switching mechanism is deactivated. Similarly, upon activation of the switching mechanism to operate one of the vaporizers 150, the humidification component 450 and/or other vaporizers 150 may be prevented from operating until the switching mechanism is deactivated.

The switching mechanism 1020 may include one or more of a gas flow diverter, bi-stable switch, pneumatic switch, rotary switch, lever, knob or other actuator which is operable by a user. For example, the switching mechanism 1020 may include each of the vaporizer 150 and the humidification component 450 being operable by a switch. The switch may be a mechanical or electronic switch. The switch may be a rotary switch operable by a user through rotation of a dial 158 on the vaporizer 150 and a dial 428 on the humidification component 450 as described in relation to FIGS. 46 to 51.

FIG. 53 is a schematic diagram showing mechanically interlocked switches for a vaporizer 150 and humidification component 450 in the respiratory apparatus 100, according to some embodiments of the disclosure. In this embodiment, the flow source 1030 includes the gas delivery apparatus 1040 which is supplied source gas via the gas supply 1060. The source gas includes NO, O2 and air, which are then delivered through the gas mixing element 1042 and/or flow meters 1090 to provide the desired composition and flow of the breathing gases in the respiratory apparatus 100. The flow of breathing gases then may pass through either a vaporizer 150 or a humidification component 450 depending on which switch 158 or 428 is operable. The switches of the vaporizer 150 and the humidification component 450 may be linked together to prevent simultaneous operation of the humidification component 450 and the vaporizer 150.

The switching mechanism 1020 may also be coupled with the interlocking mechanism of the respiratory apparatus 100 as described in relation to FIGS. 46 to 51. Upon activation of the switching mechanism 1020 to operate the humidification component 450, the interlocking mechanism enables operation of the humidification component 450 and disables operation of the vaporizer 150 due to the mechanically interlocked switches. Upon activation of the switching mechanism 1020 to operate the vaporizer 150, the interlocking mechanism enables operation of the vaporizer 150 and disables operation of the humidification component 450 due to the mechanically interlocked switches.

Enabling and disabling of each of the humidification component 450 and the one or more vaporizers 150 may occur through a variety of suitable means and in some embodiments, may involve electronic and/or mechanical actuators. These actuators may be operatively linked to the switches and interlocks discussed in relation to FIGS. 42 to 51 which enable selective operation of the humidification component 450 and the at least one vaporizer 150. For example, electronic actuators controlled by the interlocking and switching features may cause power supply to the humidification component 450 to be altered e.g. to reduce humidity to zero or a low/negligible amount. In another example, electronic actuators controlled by the interlocking and switching features may cause power supply to the one or more vaporizers 150 to be altered e.g. to reduce release of anaesthetic agents to zero or a low/negligible amount. In another example, mechanical actuators controlled by the interlocking and switching features may comprise one or more valves, such as shut off or diverter valves, that can reduce or obstruct the flow path into or from the component. Alternatively or additionally, one or more solenoids or proportional valves may be provided downstream or at the outlet of the humidification component 450 and/or the vaporizer 150 (or at the inlet of the vaporizer) to enable/disable these components by increasing or decreasing flows. In other embodiments, when the vaporizer 150 is disabled, the vaporizer may still form part of the flow path through which gases are delivered to the patient although it may, in other cases be closed by use of a shut off valve, solenoid or the like as discussed previously.

FIG. 54 is a schematic diagram of the three gas lines supplied to the respiratory apparatus 100 and where the respiratory apparatus 100 including a plurality of flow meters 190 in some embodiments. In FIG. 54, the respiratory apparatus 100 is supplied with sources of gas including nitric oxide (NO), oxygen (O2) and/or air, such as via the flow source 1030 or gas source 1060, as illustrated in FIG. 38. The NO gas line includes a flow meter 190A and the air gas line includes a flow meter 190B to control the flow rate for gas mixing to the desired composition for delivery of the flow of breathing gases to the patient 300. In this embodiment, the O2 gas line includes a flow meter 190C and a flow meter 190D. The operation of the flow meters 190C and 190D in the oxygen line are controlled by a switching mechanism 700.

When the respiratory apparatus 100 is operated in the first mode, which may be an anaesthetic ventilation mode (e.g., providing ventilatory support with or without anaesthetic agents), a switching mechanism 700 disables operation of the O2 gas line having the flow meter 190D. In contrast, when the respiratory apparatus 100 is operated in the second mode, which is ideally a high flow respiratory support mode, the switching mechanism 700 disables operation of the flow meter 190C and enables operation of the flow meter 190D for delivering O2 and/or air at a fixed flow rate to the patient 300. The flow rate may be in the range of about 20 L/min to about 90 L/min. The flow rate may be in the range of about 40 L/min to about 70 L/min. The flow rate may be about 20 L/min, about 40 L/min or about 70 L/min.

FIG. 55 is another schematic diagram of the three gas lines supplied to the respiratory apparatus 100, including the three flow meters 190A, 190B and 190C, and including two additional flow meters 190E and 190F operable on the O2 gas line. When the respiratory apparatus 100 is operated in the second mode, which is ideally a high flow respiratory support mode, the switching mechanism 700 disables operation of the flow meter 190C and enables operation of one of the flow meters 190E and 190F. As indicated in FIG. 55, the flow meter 190E may deliver O2 at a flow rate of about 40 L/min to the patient 300 and the flow meter 190F may deliver O2 at a flow rate of about 70 L/min to the patient 300.

FIGS. 56 and 57 illustrate gas flow through the respiratory apparatus 100 in two modes of operation which are controlled by the switching mechanism 1020 as previously described. The switching mechanism 1020 may be operable in conjunction with the interlocking mechanism described in relation to FIGS. 41 to 46 to change the mode of operation of the respiratory apparatus 100. The schematic diagrams shown are greatly simplified from that of FIG. 38 and exclude the vaporizer 150 in the inspiratory flow path to the inspiratory conduit 110, and exclude components in the expiratory flow path, including the rebreathing components 140.

FIG. 56 illustrates the first mode of operation of the respiratory apparatus 100 in which the respiratory apparatus 100 delivers breathing gases to the inspiratory flow path and receives return of expired gases via the expiratory flow path. The respiratory apparatus 100 receives a fresh gas flow (FGF) which is the mixture of gases (e.g., one or more of NO, O2 and air) and/or one or more anaesthetic agents which replenish gas flow through the rebreathing components 140. The gas flow is directed to the inspiratory conduit 110 for delivery of the flow of breathing gases to the patient's airway 310 via the first patient interface 120 (see also FIG. 38). Expired gases from the patient 300 are received via the expiratory conduit 130 forming a return path 140 with the CO2 absorber 141 configured to treat returned expired gas from the patient 300 before recirculating to the patient 300 in the first mode.

In FIG. 57, the switching mechanism 1020 allows the respiratory apparatus 100 to be operated in the second mode of operation in which the respiratory apparatus 100 delivers breathing gases to the inspiratory flow path at a pre-determined flow rate without return of expired gases from the patient 300. In the second mode, the respiratory apparatus 100 receives a fresh gas flow (FGF) which is a mixture of O2 and/or air without any anaesthetic agents (volatile or non-volatile). The gas flow is directed to the humidification component 450 to condition the flow of breathing gas to the pre-determined humidity and/or temperature before delivery to the patient 300. The conditioned flow of breathing gas is then delivered through the inspiratory conduit 210 to the patient 300 via a second patient interface 220. Alternatively, in the second mode, the conditioned flow of breathing gas may be delivered directly from the humidification chamber 450 to the inspiratory conduit 210 and not be returned to the respiratory apparatus 100.

In the second mode of operation, the CO2 absorber 141 may be further configured to condition the breathing gases in the inspiratory flow path to a pre-determined temperature and/or humidity before delivery to the patient 300. The CO2 absorber 141 in the second mode may be configured to condition the breathing gases to the pre-determined temperature and/or humidity by one or both of changing an amount of soda lime present in the CO2 absorber 141, and changing an amount of CO2 provided to the soda lime present in the CO2 absorber 141.

For example, the reaction of the soda lime CO2 absorber 141 could be utilised to humidify a high flow of gases. A switch could be employed in the respiratory apparatus (mechanical, electronic or otherwise), to switch the CO2 absorber 141 between the anaesthetic ventilation mode and high flow mode. Furthermore, the geometry of the soda lime may be adjusted to change the level of humidification and increase the humidity to achieve suitable levels for high flow respiratory support. Additionally/alternatively, the amount of CO2 provided to the soda lime could be adjusted to increase the reaction with the soda lime and thus, increase the humidity. Accordingly, the CO2 absorber 141 could be employed in the second mode (e.g., high flow mode) of the respiratory apparatus 100 to provide additional humidification of the flow of gases as shown in FIG. 57.

The two modes of operation illustrated in FIGS. 56 and 57 may be controlled through detection of coupling of the humidification component 450 with the mount 160. The respiratory apparatus 100 may be configured to detect coupling of the humidification component 450 with the mount 160 to enable operation of the respiratory apparatus 100 in the second mode. As illustrated in FIG. 38, the respiratory apparatus 100 may include a sensor 1050 in communication with a controller 1010. The sensor 1050 may be configured to detect coupling of the humidification component 450 with the mount 160. The controller 1010 may be configured to process data from the sensor 1050 to distinguish between coupling of a humidification component 450 and a vaporizer 150. The controller 1010 may then communicate with the switching mechanism 1020 to switch between the modes of operation such as between the first mode and the second mode as illustrated in FIGS. 56 and 57.

The sensor 1050 may include an electronic and/or mechanical feature for detecting the coupling. For example, the sensor 1050 may detect a change in an electrical property, such as resistance, capacitance or inductance in an electrical circuit when the humidification component 450 is coupled with the mount 160. Alternatively, the humidification component 450 may include a machine-readable component associated with the housing 422, such as a radio-frequency identification detection (RFID) device which is able to be detected by the sensor 1050 upon coupling of the humidification component 450 with the mount 160. In other embodiments, the respiratory apparatus may exclude a sensor 1050 and instead provide a mechanical arrangement for detecting the coupling, such as through a lock and key arrangement or the like, as would be appreciated by a person skilled in the art.

### Advantages

Embodiments of the present disclosure provide configurations that allow easy interchange between respiratory support via high flow, and respiratory support by anaesthetic ventilation/anaesthetic agent delivery via the anaesthetic machine.

Some embodiments provide a single machine that provides the functionality of both high flow respiratory support and a rebreathing system for delivery of anaesthesia, which can be easily transitioned or switched between a high flow mode and an anaesthesia machine (rebreathing) mode. Desirably, this allows a clinician to easily switch between deployment of high flow respiratory support and a rebreathing system and then subsequently inducing/ventilating the patient. This reduces the overall number of components, simplifying the working environment and making it easier for an anaesthetist to perform required tasks during procedures involving high flow therapies in addition to administration of anaesthesia.

When switching to a high flow mode from a rebreathing mode, for safety reasons it is desirable that the transition stops the delivery of gases from the anaesthesia machine. Uncontrolled delivery of O₂ outside of the re-breathing system (e.g. when the mask is removed from the patient) could pose a fire risk, and there could be wastage of anaesthetic gas, as well as unintentional release of anaesthetic gases to the operating environment which could not only contaminate the high flow respiratory support gases, but impact the performance of individuals in the operating environment. Embodiments of the present invention address one or more of these problems.

Also described herein are various embodiments, apparatuses, connectors, assemblies, accessories, devices and the like for achieving switching between modes of respiratory support. Some of these provide the convenience of controlling mode selection when the user is not situated at the “machine” by providing switching actuators closer to the patient, or the clinician. Some embodiments provide for automatic selection of modes of operation by monitoring characteristics of gases in the system, such as gas pressure and CO₂ concentration in expired gases. Not only do these features improve convenience and operability of a system that provides these modes of respiratory support, they also have the capability to improve patient safety.

Humidification of breathing gases delivered during high flow respiratory support can be important because without it, gases may have a drying effect on the airways which can lead to damage and other complications. Embodiments of the present disclosure provide a respiratory apparatus that allows for humidification of breathing gases that are delivered during high flow respiratory support. The respiratory apparatus includes a mount that is couplable with a humidification component for conditioning a flow of breathing gases to a pre-determined temperature and/or humidity before delivery to the patient.

Embodiments of the respiratory apparatus may also advantageously provide an interlocking mechanism to prevent simultaneous operation of the humidification component and one or more vaporizers. This may desirably prevent delivery of volatile anaesthetic agents to the patient during high flow respiratory support. Furthermore, the respiratory apparatus may also include a switching mechanism configured to enable selective operation of the humidification component and the at least one vaporizer. The switching mechanism which may be coupled with the interlocking mechanism to provide for easy of switching and safe transition between the anaesthetic ventilation mode and high flow mode of the respiratory apparatus during medical procedures requiring both forms of respiratory support.

It is to be understood that various modifications, additions and/or alternatives may be made to the parts previously described without departing from the ambit of the present invention as defined in the claims appended hereto.

The invention may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth.

Where any or all of the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that the following claims are provided by way of example only, and are not intended to limit the scope of what may be claimed in future. Features may be added to or omitted from the claims at a later date so as to further define or re-define the invention or inventions.

1. A multi-lumen assembly for use with a respiratory support system, the multi-lumen assembly having a plurality of conduits including:
(a) a first inspiratory conduit having a first conduit inflow end couplable with a first gas outlet of the respiratory support system;
(b) a second inspiratory conduit having a second conduit inflow end couplable with a second gas outlet of the respiratory support system; and
(c) an expiratory conduit having an expiratory conduit outflow end couplable with an expired gas inlet of the respiratory support system.

2. A multi-lumen assembly according to claim 1, wherein the first inspiratory conduit has a first conduit outflow end couplable with a first patient interface configured to sealingly engage with and direct flow into an airway of the patient.

3. A multi-lumen assembly according to claim 1 or claim 2, wherein the second inspiratory conduit has a second conduit outflow end couplable with a second patient interface configured to direct flow into an airway of the patient and is a non-sealing interface.

4. A multi-lumen assembly according to any one of claims 1 to 3, wherein at least a length of the plurality of conduits is arranged co-axially.

5. A multi-lumen assembly according to any one of claims 1 to 4, including a mechanism to retain at least a length of the plurality of conduits in a group.

6. A multi-lumen assembly according to claim 5 wherein the mechanism includes a webbing arranged between at least pairs of the plurality of conduits at intervals or continuously along at least a length of the plurality of conduits.

7. A multi-lumen assembly according to claim 6, wherein the webbing is frangible to facilitate separation of at least a length of one or more of the plurality of conduits from the group.

8. A multi-lumen assembly according to any one of claims 5 to 7, wherein the mechanism includes a sheath applied around the plurality of conduits.

9. A multi-lumen assembly according to claim 8, wherein part of the sheath is removeable.

10. A multi-lumen assembly according to claim 8 or claim 9, wherein the sheath provides a smooth outer surface.

11. A multi-lumen assembly according to any one of claims 5 to 7, wherein the mechanism includes one or more retainers configured to retain two or more conduits in the plurality of conduits in a group.

12. A multi-lumen assembly according to claim 11, wherein the retainers are slidable along a length of one of more of the conduits in the plurality of conduits.

13. A multi-lumen assembly according to any one of claims 1 to 12, wherein the first inspiratory conduit is configured to deliver breathing gases including an anaesthetic agent to the patient.

14. A multi-lumen assembly according to any one of claims 1 to 13, wherein the expiratory conduit is configured to return expired gases from the patient to the respiratory support system.

15. A multi-lumen assembly according to any one of claims 1 to 14, wherein the second inspiratory conduit is configured to deliver breathing gases to the patient at a flow rate between 20 L/min and 90 L/min.

16. A multi-lumen assembly according to any one of claims 1 to 15, including a flow switching mechanism operable to direct flow of breathing gas into the first inspiratory conduit or into the second inspiratory conduit.

17. A multi-lumen assembly according to claim 16, wherein the flow switching mechanism is a flow diverter.

18. A multi-lumen assembly according to claim 16 or claim 17, wherein the flow switching mechanism is operable by a user.

19. A multi-lumen assembly according to claim 16 or claim 17, wherein the flow switching mechanism is operatively linked with a respiratory support system controller.

20. A multi-lumen assembly according to claim 19, wherein the respiratory support system controller controls the respiratory support system to deliver breathing gases to the multi-lumen assembly according to operation of the flow switching mechanism by the user.

21. A multi-lumen assembly according to claim 19, wherein the respiratory support system controller controls operation of the flow switching mechanism.

22. A multi-lumen assembly according to any one of claims 1 to 21, wherein the outflow end of the first inspiratory conduit and an inflow end of the expiratory conduit form a common gas flow pathway defined by a single gas exchange conduit which is couplable with a first patient interface.

23. A multi-lumen assembly according to claim 22, wherein the multi-lumen assembly includes a taper to reduce the overall cross section of the assembly in a region of the single gas exchange conduit.

24. A multi-lumen assembly according to any one of claims 1 to 23, wherein the multi-lumen assembly includes a patient-end connector for coupling:
(a) an outflow end of the first inspiratory conduit with a first patient interface; and
(b) an outflow end of the second inspiratory conduit with a second patient interface.

25. A multi-lumen assembly according to claim 24, wherein the patient end connector includes a switching element operable to switch the connector between a first mode of operation in which the connector directs breathing gas to the first patient interface, and a second mode of operation in which the connector directs breathing gas to the second patient interface.

26. A multi-lumen assembly according to claim 25, wherein the switching element is operatively couplable with a respiratory support system controller.

27. A multi-lumen assembly according to claim 25, wherein the switching element is operable by a user, and the controller controls operation of the respiratory support system according to operation of the switching element by the user.

28. A multi-lumen assembly according to claim 26 or claim 27, wherein the respiratory support system controller controls operation of the switching element.

29. A multi-lumen assembly according to any one of claims 1 to 28, further including a gas sampling conduit for monitoring one or more characteristics of gas.

30. A multi-lumen assembly according to claim 29, wherein said characteristics are used by a respiratory support system controller to determine if the connector is delivering breathing gas to the patient in the first inspiratory conduit or the second inspiratory conduit and wherein the controller operates the respiratory support system to automatically select a corresponding mode of operation of the respiratory support system.

31. A system for delivering breathing gases to a patient, the system including:
(a) a flow source configured to provide gas flows through the system in a gas delivery circuit;
(b) a gas delivery circuit including an inspiratory gas flow path and an expiratory gas flow path,
the system configured to switch between a first mode and a second mode, wherein:
in the first mode, the system is operable to deliver the breathing gases to the patient via the inspiratory gas flow path and a first patient interface in fluid communication with the inspiratory flow path, and to deliver expiratory gases from the patient via the expiratory gas flow path and a second patient interface in fluid communication with the expiratory gas flow path, the breathing gases including a first flow parameter; and
in the second mode, the system is operable to deliver the breathing gases to the patient via the inspiratory gas flow path and a third patient interface, the breathing gases including a second flow parameter.

32. The system of claim 31, wherein the first flow parameter is different from the second flow parameter.

33. The system of claim 31 or 32, wherein the first flow parameter includes a first flow rate and the second flow parameter includes a second flow rate, wherein the first flow rate is less than the second flow rate.

34. The system of any one of claims 31 to 33, wherein the first flow rate is less than 15 L/min and the second flow rate is greater than 15 L/min.

35. The system of claim 34, where the second flow rate is in the range of between about 20 L/min and about 90 L/min, optionally between about 40 L/min and about 70 L/min.

36. The system of any one of claims 31 to 35, wherein the first patient interface includes a non-sealing patient interface, optionally a nasal cannula, and the second patient interface includes a sealing patient interface, optionally a mask.

37. The system of any one of claims 31 to 36, wherein the third patient interface includes a non-sealing patient interface, optionally a nasal cannula.

38. The system of any one of claims 31 to 37, wherein the expiratory flow path is inoperable in the second mode.

39. The system of any one of claims 31 to 38, wherein the first and third patient interfaces are the same.

40. The system of claim 39, wherein the system is in the first mode when the first and second patient interfaces are applied simultaneously to the patient and is in the second mode when only the third patient interface is applied to the patient.

41. The system of claim 40, wherein first and third patient interfaces include a non-sealing nasal cannula and the second patient interface includes a sealing mask, and wherein the system is in the first mode when the nasal cannula and mask are applied to the patient and the system is in the second mode when only the nasal cannula is applied to the patient.

42. The system of claim 41, wherein the mask is configured to seal over the nasal cannula and with the patient.

43. The system of any one of claims 31 to 42, the system including a third mode and a fourth patient interface wherein the system is configured to deliver the breathing gases to the patient via the inspiratory gas flow path and the fourth patient interface and to deliver expiratory gases from the patient via the expiratory gas flow path and the fourth patient interface, the breathing gases including a third flow parameter.

44. The system of claim 43, wherein the fourth patient interface includes sealing patient interface, optionally an invasive patient interface, and optionally an endotracheal tube.

45. The system of any one of claims 31 to 44, wherein the first flow parameter includes a pressure and/or volume parameter.

46. The system of claim 43 or 44 or claim 45 when dependent on claim 43 or 44, wherein the third flow parameter includes one or more of a flow rate, pressure or volume parameter.

47. The system of claim 45 or 46, wherein the system is configured to control delivery of breathing gases to the patient based on pressure and/or volume.

48. The system of any one of claims 31 to 47, the system including an inspiratory conduit defining at least a portion of the inspiratory gas flow path and an expiratory conduit defining at least a portion of the expiratory gas flow patient, and a common connector provided at an end of the inspiratory and expiratory conduits, the common connector configured to connect to one or more patient interfaces.

49. The system of any one of claims 31 to 48, the system including a controller in communication with the flow source, and one or more of a sensor and an input interface in communication with the controller to provide an input to the controller to control the flow source to provide flow of breathing gases in the first or second mode.

50. The system of claim 49 when dependent on any one of claims 43 to 47, wherein the sensor and/or input interface is configured to provide an input to the controller to control the flow source to provide flow of breathing gases in the third mode.

51. The system of any one of claims 31 to 50, the system including a humidifier, wherein the breathing gases are heated and humidified by the humidifier in the second mode prior to being delivered to the patient.

52. The system of any one of claims 31 to 50, wherein the expiratory gases from the patient are returned to the inspiratory gas flow path in the first mode.

53. The system of any one of claims 31 to 47 or claims 48 to 52 when dependent on any one of claims 31 to 47, where in the expiratory gases from the patient are returned to the inspiratory gas flow path in the third mode.

54. The system of claim 52 or 53, the system including a CO₂ remover configured to remove CO₂ from the expiratory gases before returning the expiratory gases to the inspiratory gas flow path.
